(19) United States
(12) Patent Application Publication
(10) Pub. No.: US 2023/0218745 A1
(43) Pub. Date: Jul. 13, 2023

(54) MODIFIED ALPHAVIRUS FOR USE AS COVID-19 VACCINE

(71) Applicant: New York University, New York, NY (US)

(72) Inventors: Daniel MERUELO, Scarborough, NY (US); Silvana OPP, New York, NY (US); Antonella SCAGLIONE, San Diego, CA (US); Christine PAMPENO, New York, NY (US); Alicia Hurtado MARTINEZ, New York, NY (US); Ziyan LIN, Long Island City, NY (US)

(21) Appl. No.: 18/000,772

(22) PCT Filed: May 28, 2021
(86) PCT No.: PCT/US2021/034842

§ 371 Date: Dec. 5, 2022

Related U.S. Application Data
(60) Provisional application No. 63/034,791, filed on Jun. 4, 2020.

(51) Int. Cl. A61K 39/215; C12N 15/86; A61P 31/14; C07K 14/165; C07K 16/28; C07K 14/54

(52) U.S. Cl. CPC A61K 39/215; A61P 31/14; C07K 14/165; C07K 14/5434; C07K 16/2878; C12N 15/86; A61K 2039/5256

(57) ABSTRACT

Modified alphaviruses encoding a SARS-CoV-2 spike protein or antigenic segment of the SARS-CoV-2 spike protein are provided. The modified alphaviruses include replicative defective Sindbis viruses. The modified viruses express or are administered with an immunomodulatory agent that is an agonist antibody or antigenbinding fragment thereof, or a cytokine, or a combination thereof. Pharmaceutical compositions that include the modified alphaviruses and methods of using the modified alphaviruses and compositions that contain them are provided. The compositions are used to stimulate a therapeutic or protective effect against SARS-CoV-2 infection that includes humoral and cell mediated responses.

### CROSS-REFERENCE TO RELATED APPLICATIONS

This application claims priority to U.S. Provisional Application No. 63/034,791 filed on Jun. 4, 2020.

### STATEMENT REGARDING FEDERALLY SPONSORED RESEARCH

This invention was made with government support under grant number 5R44CA206606 awarded by the National Institutes of Health. The government has certain rights in the invention.

### REFERENCE TO SEQUENCE LISTING

This application includes an electronically submitted sequence listing in .txt format. The .txt file contains a sequence listing entitled “Sindbis_Coronavirus_ST25.txt” created on May 28, 2021 and is 460 bytes in size. The sequence listing contained in this .txt file is part of the specification and is hereby incorporated by reference herein in its entirety.

### FIELD

The present disclosure relates generally to alphavirus-based vaccines that are to stimulate immune responses against SARS-CoV-2. The vaccines express one or more SARS-CoV-2 antigens and can express additional immunomodulatory agents.

### BACKGROUND

Traditionally, vaccines have been designed to induce antibody responses and have been licensed on their capacity to induce high titers of circulating antibody to the pathogen[1]. With increased knowledge of host-virus interactions, it has become clear that the cellular arm of the immune response is also crucial to the efficacy of vaccines against pathogens and to provide appropriate help for antibody induction. Various strategies have emerged that specialize in developing candidate vaccines that solely induce either cellular or humoral responses[1]. However, as most viruses and pathogens reside at some point during their infectious cycle in the extracellular as well as intracellular space, vaccines need to promptly elicit a strong T cell memory response against intracellular pathogens, so that, at the earliest stages of the infective process preventing disease can be addressed in coordination with antibodies, thus preventing disease.

An early CD4+ and CD8+ T cell response against SARS-CoV-2 is considered to be protective[2; 3]. However, this response can be difficult to generate because of efficient immune evasion mechanisms of SARS-CoV-2 in humans[4], especially in the elderly. Immune evasion by SARS-CoV-2 is likely exacerbated by reduced myeloid cell antigen presenting cell (APC) function and B cell decline in older adults. In such cases, late T cell and uncoordinated adaptive immune responses may instead amplify pathogenic inflammatory outcomes in the presence of sustained high viral loads in the lungs, leading to death.

In the ongoing COVID19 pandemic, vaccines play a key role in the strategy to bring SARS-CoV-2 transmission under control. Safety and eliciting a broad-spectrum immune response are paramount for coronavirus vaccine development. Data from vaccine clinical trials and real-world evidence show that available coronavirus vaccines are able to cut the risk of severe COVID19 disease and transmission. However, even with first generation vaccines currently being globally administered to reduce transmission and severity of the disease, the emergence of circulating variants has raised major concerns that challenge sustained vaccine efficacy, particularly in the face of waning immunity following vaccination[5; 6; 7; 8; 9; 10; 11]. Recent data have indicated that escape (appearance and spread of viral variants that can infect and cause illness in vaccinated hosts) protection by vaccines designed against the Wuhan-1 strain is inevitable[8].

The global COVID19 pandemic is unlikely to end until there is an efficient pan-global roll-out of SARS-CoV-2 vaccines. Though multiple vaccines are currently available, vaccine rollout and distribution at the time of writing this paper is quite incomplete. The three largest countries in the western hemisphere- US, Brazil, and Mexico - have vaccinated 32.7%, 7%, and 6.6% of their populations, respectively, compared to only 2.2% in India [12]. Vaccine distribution to date has been highly non-uniform among these and other countries around the globe, encountering many challenges. Unequal vaccine roll-out and the new B.1.617 variant are highly concerning. Major challenges have been supplies shortages, logistical problems, complex storage conditions, priced affordably, and safety[13]. Consequently, the pandemic is currently sweeping through India at a pace faster than ever before. The countries’ second wave became the worst COVID19 surge in the world, despite previous high infection rates in megacities that should have resulted in some immunity. More cost-effective and facilitated delivery of broad-spectrum SARS-CoV-2 vaccines would help improve wide and rapid distribution, which would in turn minimize vaccine-escape. Thus, there is an ongoing and unmet need for improved compositions and methods for combating SARS-CoV-2. The present disclosure is pertinent to these needs.

### SUMMARY

The present disclosure provides a modified Alphavirus and populations of the same for use in prophylaxis and/or therapy for SARS CoV-2 infection. The modified Alphavirus platform is illustrated by way of a novel modified Sindbis Virus (SV) vaccine encoding and transiently expressing the SARS-CoV-2 spike protein (SV.Spike), which induces a strong adaptive immunity that fully protects transgenic mice that express the SARS-CoV receptor (human angiotensin-converting enzyme 2 [hACE2]), K18-hACE2, against live SARS-CoV-2 virus infection. To additionally increase safety, the disclosure provides replication-deficient vectors that only transiently express the encoded antigen and other immunomodulatory proteins of interest. The disclosure demonstrates that a combination of the described vaccine with αOX40 agonistic antibodies significantly enhances the induction of immunity by the SV.Spike vector. Specifically, seroconversion and abundance of IgG neutralizing antibodies and T cell immunity through early initiation of Th1-type T cell polarization are markedly augmented to potentiate long-term immunity protective against SARS-CoV-2 infection in mice. The disclosure accordingly provides a safe and effective vaccine platform that provides humoral and cellular immunity to the SARS-CoV-2 spike. This platform has the potential to be applied to other emerging pathogens.

The disclosure includes compositions comprising the described modified alphaviruses, plasmids encoding the vaccine components, isolated polynucleotides, isolated viral particles, methods of making the described vaccines, and kits for producing the described vaccines.

### BRIEF DESCRIPTION OF THE FIGURES

FIG. 1. Characterization of Sindbis carrying the SARS-CoV-2 spike. (A) Schema of SARS-CoV-2 spike gene cloned into Sindbis vector system. (B) Western Blot of SARS-CoV-2 spike produced from the Sindbis vector. Lanes shown are titration of the vector, and recombinant spike control produced in HEK cells. (C) Schematic of vaccination. C57BL/6 mice were immunized with 1x 0.5 ml SV.Spike/and or αOX40 antibody (250 µg/dose) on day 0. A boost injection of SV.Spike/and or αOX40 were once given on day 14. On day 7,14 and 21, 75 and 100, blood was taken to determine Sars-Cov-2 spike specific antibodies by ELISA. Spleens were excised and a single cell suspension was stained and analyzed by flow cytometry. T cells were isolated and were used for ELISPOT assay and Seahorse. As control, naïve C57BL/6J mice were used.

FIG. 2. SARS-CoV-2 spike specific antibodies induced by Sindbis. Characterization of serum IgA, IgM, and IgG in C57BL/6J mice vaccinated with SV.Spike at day 21, 75 and 100 post-immunization. (A) The levels of Spike-specific IgA, IgM, and IgG isotypes in sera of immunized mice at different time windows. P values were calculated by one-way ANOVA with the Bonferroni correction in Graphpad Prism. n.s. > 0.05; **P < 0.01; ***P < 0.001; ****P<0.0001. (B) The kinetics of Spike-specific IgA, IgM, and IgG isotypes in sera of immunized mice at different time windows. Two-way ANOVA with the Bonferroni correction in GraphPad Prism used to calculate the indicated P values. The data presented are the mean of three technical replicates. The median values of (A) OD450 or (B) calculated log2 antibody levels were plotted for each isotype of three antibodies.

FIG. 3. Blockade of SARS-CoV-2 Spike-ACE2 binding and spike protein-mediated cell-cell fusion by anti-SARS-CoV-2 Spike neutralizing antibodies. (A, B) In the assay, anti-SARS-CoV-2 neutralizing antibodies from immunized C57BL/6J mice, block recombinant Spike protein from binding to the hACE2 protein pre-coated on an ELISA plate. Percentage of inhibition distributed along y-axis of SARS-CoV-2 Spike-hACE2 interaction for the indicated reciprocal plasma dilutions by mouse sera collected at (A) 21 and (B) 75 days post vaccination with Sindbis expressing Sars-CoV-2 Spike (SV.Spike), SV.Spike in combination with αOX40 and αOX40 alone compared to the naive group. Area under the curve (AUC) values of serum antibodies were calculated from reciprocal dilution curves in antibody detection assay. The data presented are the mean of 5 biological replicates with two technical replicates. Statistics were performed using a One-way ANOVA with the Bonferroni correction in Graphpad Prism. n.s. > 0.05; *P < 0.05; **P < 0.01; ***P<0.001; ****P<0.0001. (C) Images of SARS-CoV-2 Spike-mediated cell-cell fusion inhibition on 293T/ACE2 cells by sera from C57BL/6J vaccinated mice. SARS-CoV-2 Spike-transfected 293 T were incubated with mice serum at 1:100 dilution and applied onto HEK293T-ACE2 cells for 24 hours. Scale bar, 100 mm. (D) Quantification of the number aggregates (left panel) and inhibition of cell-cell fusions (right panel) induced by SARS-CoV-2 Spike following pre-incubation with naive, SV.Spike, SV.Spike+αOX40 and αOX40 alone are shown. N = 5 biological replicates with 2 independent technical replicates. One-way ANOVA with Bonferroni correction *P < 0.05, **P < 0.01, and ***P < 0.001. (E) Representative confocal images of 293 T/ACE2 cells treated with serum from Naive and SV.Spike+αOX40-immunized mice pre-incubated with SARS-CoV-2 Spike recombinant protein and stained for ACE2 (green), SARS-CoV-2 Spike (red), and DAPI (blue). Scale bar: 20 µm.

FIG. 4. SV.Spike vaccine prevents infection of SARS-CoV-2 in hACE2 transgenic (hACE2-Tg) mice. Luciferase-encoding SARS-CoV-2 Spike pseudotyped lentivirus was incubated with mouse sera collected at (A) 21 and (B) 75 days post vaccination with SV.Spike, SV.Spike in combination with αOX40 and αOX40 antibody alone compared and unvaccinated naive groups. Area under the curve (AUC) values of serum antibodies were calculated from reciprocal dilution curves in antibody detection assay. The data presented are the mean of 5 biological replicates with two technical replicates. Statistics were performed using a One-way ANOVA with the Bonferroni correction in GraphPad Prism. n.s. > 0.05; ****P<0.0001. (C) Expression of pseudotyped Sars-CoV-2-Spike-lacZ lentivirus in whole mouse lung following intranasal delivery. One week following vector nasal administration to the right nostril of four weeks old hACE2 transgenic mice (B6(Cg)-Tg(K18-ACE2)2Prlmn/J), expression of lacZ was analyzed in mice airways. X-Gal stained whole lungs from (left) hACE2 non carrier control mouse and (right) hACE2 transgenic mouse, both dosed with Sars-CoV-2-Spike-lacZ pseudotyped lentivirus. (D) Schematic of the re-challenge experiment with SARS-CoV-2-Spike-lacZ lentivirus. (E) On day 21 (upper panels) and 75 (lower panels) after the initial infection hACE2-Tg were rechallenged with 3.6 x 10⁵ PFU of Sars-CoV-2-Spike-lacZ pseudotyped lentivirus and then analyzed for X-Gal staining at day 7 post rechallenge. Three non-vaccinated naive animals were included as a positive control in the rechallenge experiment. (F-H) hACE2-Tg mice were vaccinated with SV.Spike and/r αOX40 and challenged with 10⁴ particles of live SARS-CoV-2 coronavirus at day 21 post immunization. Weight loss and mortality was observed daily for 14 days after live virus infection and compared to the naïve unvaccinated group. (G) Change of body weight during systemic infection with SARS-CoV-2 coronavirus. Percent weight loss (y-axis) is plotted versus time (x-axis). Data points represent mean weight change +/- SEM. (H) Survival curves of SV.Spike with or without αOX40 treated and naive unvaccinated mice. n = 5 mice per group.

FIG. 5. SV.Spike in combination with αOX40 activates and metabolically reprograms T cells. C57BL/6J mice were vaccinated with first doses of SV.Spike and/or αOX40. Naive mice were used as control. T cells were isolated from spleens on day 7 or otherwise indicated. (A) Mitochondrial respiration was assessed by measuring the median values of oxygen consumption rates (OCR) in T cells of indicated groups using an extracellular flux analyzer. Oligomycin, FCCP, Antimycin A and Rotenone were injected as indicated to identify energetic mitochondrial phenotypes. (B) Energy Map (OCR versus ECAR) of T cells from naive or mice treated with SV.Spike, or αOX40 or combination of SV.Spike+αOX40 on day 7. (C) Baseline extracellular acidification rates (ECAR) in T cells of indicated groups. (D) ATP Production in T cells of indicated groups. (E-J) Splenocytes were analyzed by flow cytometry. (E, F) Expansion of CD4+ (E) and CD8+ T (F) cells is indicated by expression of Ki67-positive cells. (G, H) Activation of CD4+ T cells (G) and CD8+ T cells (H) indicated by CD38+ expression. (I, J) Expression of CD44+ positive cells. CD4 (I) and CD8 (J) cells. Error bars indicate SEM. Results are representatives of two independent experiments. Each symbol represents an individual mouse in E, F, G, H, I, J. Bars represent means. Statistical significance was determined with the Kruskal-Wallis test followed by the Dunns’ test. n.s. > 0.05, **p<0.005, * * *p≤ 0.001.

FIG. 6. Sindbis expressing SARS-CoV-2 spike+αOX40 C57BL/6J vaccinated mice are characterized by a unique transcriptional signature of T cells. Combination therapy markedly changes the transcriptome signature of T cells favoring T cell differentiation towards effector T cells with a Th1 type phenotype 7 days after prime vaccination. (A) Principal component analysis (PCA) of RNA seq data from naive, SV.Spike and/or αOX40 groups. (B) Venn diagrams summarizing the overlap between differentially expressed genes (DEGs) from SV.Spike (blue), αOX40 (pink) and SV.Spike+αOX40 (purple). Up-regulated DEGs (left) and down-regulated (right). (C) MA plots of differentially expressed genes in T cells of naive versus SV.Spike (top graph), αOX40 (middle graph) and combination (bottom graph). Significantly (p<0.05) upregulated and downregulated DEGs are depicted in red or blue, respectively. (D) Pathway and network analysis based on GSEA in T cells isolated from mice treated with combination therapy. Downregulated (blue circle) and upregulated (red circles) pathways are shown, respectively. (E) Pathway and network analysis based on GSEA in T cells isolated from mice treated with single dose of SV.Spike. Top 10 hub biological process gene ontology (GO) terms ranked by the Cytoscape plugin cytoHubba (red, highest ranks; yellow, lowest ranks) in the SV.Spike only (F) versus combination immunized group (G). Heatmap analysis of selected genes based on normalized read counts linked to T cell differentiation in the SV.Spike and/or αOX40 immunized mice compared to naive (H). Highlighted selected gene set enrichment analysis (GSEA) pathways based on DEG in naive versus SV.Spike (I) and combination treated group (J).

FIG. 7. Reprogrammed T cells in SV.Spike+αOX40 vaccinated mice display enhanced Th-1 T cell phenotype mediated cytokine production and cytotoxic T cell activity. Spleens of naive and C57BL/6J vaccinated mice were excised on day 7 after prime vaccine doses for flow cytometry analysis (A-J). T cells were further isolated for (K) Interferon-gamma (IFNγ) enzyme-linked immunospot analysis (ELISpot) and (L, M) cytotoxicity analysis. Percentage of (A) CXCR3 and (B) CX3CR1 expressing CD4+ T cells indicating Th1-like T cell effector phenotype. (C) Percentage of Tbet+ICOS+ positive Th1-type effector CD4+ T cell polarization. (D) Representative blots. (E) Percentage of granzyme B (GrB) positive CD4+ T cells from indicated groups using flow cytometry. (F) Representative plots. (G) Percentage of GrB positive CD8+ T cells from indicated groups using flow cytometry. (H) Representative plots. (I) Percentage of Perforin positive CD8+ T cells. (J) Representative blots. Bars represent means ± SEM (A-J) and each symbol represent an individual mouse (n=5 per group). Statistical significance was determined with the Kruskal-Wallis test followed by the Dunns’ test. Results are representatives of at least two independent experiments. (K) Amount of IFNγ spots per 10⁵ T cells determined by ELISpot. (L, M) Cytotoxic activity of T cells harvested on day 7 from control and treated mice (n = 5 mice per group). T cells were isolated from splenocytes and were co-cultured with 293T/ACE2 cells for 2 days. Effector-to-target (E/T) cell ratio (T cells/ACE2 cells) was 30:1. Cytotoxicity was determined for each group of mice by measuring the infectivity of luciferase-encoding pseudotyped particles with (L) Spike protein of SARS-CoV-2 or (M) VSV-G and is shown relative to naive T cells. Bars or symbols represent means ± SEM, and statistical significance was determined with one-way ANOVA with the Bonferroni correction. n.s. > 0.05, *p<0.05, ***p≤ 0.001, ****p ≤ 0.0001.

FIG. 8. SV.Spike in combination with αOX40 drives follicular T helper cell function and metabolic activation of B cells. C57BL/6J mice were vaccinated with SV.Spike and/or αOX40. Naive mice were used as control. T cells were isolated on day 7 after prime vaccine doses and RNAseq was performed (A). GSEA for biological processes identified pathway enrichment that regulates B cell activation after prime vaccine doses in combination immunized mice. Splenocytes were excised on day 21 for flow cytometry analysis (B-E). (B-C) CXCR5+ICOS+ expressing CD4+ T cells and (D-E) CXCR5+CD44+ expressing CD4+ T cells indicating Tfh-cell differentiation with representative plots (n=5 individual mice per group). (F-H) B cells were isolated for Seahorse metabolic flux analysis one week after boost doses. (F) Mitochondrial respiration was assessed by measuring the median values of oxygen consumption rates (OCR) in B cells of indicated groups using an extracellular flux analyzer. Oligomycin, FCCP, Antimycin A and Rotenone were injected as indicated to identify energetic mitochondrial phenotypes. (G) Energy Map (OCR versus ECAR) of B cells from naive or mice treated with SV.Spike and/or αOX40 on day 21. (H) Baseline extracellular acidification rates (ECAR) in B cells of indicated groups. Error bars indicate SEM. Results are representatives of one or two independent experiments. Bars or symbols represent means ± SEM, and statistical significance was determined with the Kruskal-Wallis test followed by the Dunns’ test. n.s. > 0.05, **p<0.005. (I) Correlation analysis of ICOS+CXCR5+ expressing Tfh cells with IgG antibody titers at 21 days post vaccination. (n=5). Pearson’s rank correlation coefficients (R) and p values are shown.

FIG. 9. Combination of SV.Spike and αOX40 promotes robust tissue specific Th1-type T cell immune response in lungs. Presence of activated T cells in lungs after 21 days after prime vaccine doses indicate tissue specific immune protection. C57BL/6J mice were immunized by a Prime/Boost strategy with SV.Spike and/or αOX40 and lungs were excised and a single cell-suspension was stained for flow cytometry analysis. Naive mice were used as control. (A) CD4+ Tfh type T cells presence in the lung indicated by ICOS+CXCR5+ double-positive CD4+ T cells. (B) Representative plots. (C) Expression of ICOS+Tbet+ double positive CD4+ T cells indicating Th-1 type effector cells polarization and recruitment to the lungs. (D) Representative plots. (E-H) Cytotoxic T cells in lungs indicated by (E) Granzyme B positive CD4+ T cells and representative plots (F) and CD8+ effector T cells indicated by GrB+ and representative plots (G, H). Bars or symbols represent means ± SEM. Each symbol represents one individual mouse. Statistical significance was determined with the Kruskal-Wallis test followed by the he Dunns’ test. n.s. > 0.05, *p<0.05, **p<0.005, ***p≤ 0.001.

FIG. 10. Combination of SV.Spike and αOX40 potentiates CD4 effector memory T cells 14 weeks after prime vaccine doses. Splenocytes from indicated immunized C57BL/6J mice groups were harvested 14 weeks after first vaccine doses. Memory phenotype was characterized in spleen from indicated groups by flow cytometry by gating on CD4+ cells. The percentage of CD4+ T cells expressing CD62L and/or CD44 was analyzed and shown (A). (B) Representative contour plots and (C) pie charts. (n=5 mice per group). TCM, central-memory T cells; TEM, effector-memory T cells.

FIG. 11. Challenging immunized mice with spike antigen promotes a fast and coordinated response of the two arms of the adaptive immune system. Humoral and T cell immune responses were assessed in vaccinated mice after rechallenge with Sindbis carrying SARS-CoV-2-Spike (SV.Spike). (A) Design steps of the rechallenge experiment in vaccination of immunized C57BL/6J mice evaluated by (B) T-cell cytotoxic assay, (C-F) Flow cytometry indicating cytotoxic CD8 T cell effector response by GrB+ positive CD8 T cells and activation of CXCR5+ICOS+ positive Tfh cells upon rechallenge, (G) binding IgA, IgM, IgG antibody ELISA to SARS-CoV-2-Spike recombinant protein (n=5 mice per group, or as otherwise indicated). Each symbol represents one individual mouse. Bars or symbols represent means ± SEM, and statistical significance was determined with one-way ANOVA with the Bonferroni correction (B, G) or with the Kruskal-Wallis test followed by the he Dunns’ test (C-F). n.s. > 0.05, **p<0.005, * * *p≤ 0.001, ****p < 0.0001.

FIG. 12. SARS-CoV-2 spike sequences cloned into the SV vector expressing. The SARS CoV-2 spike sequence originates from the BEI Resource NR-52420 plasmid. The spike sequence was cloned into the XballApal sites of the Sindbis replicon vector. The plasmid is linearized at the XhoI site, RNA is in vitro transcribed from the T7 promoter and capped. Electroporation of the replicon mRNA produces SV replicase that transcribes the spike gene from the subgenomic promoter (Psg).

FIG. 13. Dose-response curves of anti-SARS-CoV-2 spike neutralizing antibodies blocking the SARS-CoV-2 spike-ACE2 binding. Inhibition curves of SARS-CoV-2 Spike-hACE2 interaction for the indicated reciprocal serum dilutions by C57BL/6J mice sera collected at (A) 21 and (B) 75 days post vaccination with Sindbis expressing Sars-CoV-2 Spike (left panels), Sars-CoV-2 Spike (middle panels) in combination with αOX40 and αOX40 alone (right panels) compared to the naive group. Inhibition curves for mouse sera collected at 21 days post vaccination via the intramuscular (IM) route are shown in (C). The data presented are the mean of n = 5 biological replicates with n = 2 technical replicates each curve.

FIG. 14: Images of SARS-CoV and SARS-CoV-2 Spike-mediated cell-cell fusion on 293T/ACE2 cells at 6 hours (left) and 24 hours (right). HEK293T have been co-transfected with pMAX-GFP/pCDNA3.1-SARS-COV or pMAX-GFP/pCAGGS-SARS-COV-2 Spike plasmids and applied onto HEK293T and HEK293T-ACE2 cells for the indicated time points. Scramble represents expression of GFP only. Scale bar, 100 µm.

FIG. 15: Characterization of pseudotypes expressing SAR-CoV-2 Spike. Western blot analysis of the expression of p24 and Spike proteins from (A) Luciferase- and (B) nLacZ-encoding SARS-CoV-2 pseudotyped lentivirus produced in HEK293T cells transfected with pLentivirus expression plasmids as explained in the method section. A VSV-G encoding and empty (non-modified envelope) lentiviruses were also produced as expression controls. Purified SARS CoV-2 spike and p24 recombinant proteins were used as the positive controls. (C) Titration of Luciferase-encoding SARS-CoV-2 with spike protein, VSV-G and empty lentiviruses using HEK293 T/ACE2 cells. LoglO luminescence units (RLU) were measured. Titration values are expressed as TU/ml. n =3. (D) Immunofluorescence analysis of the expression of LacZ protein in HEK293 T/ACE2 cells mediated by SARS-CoV-2, VSV-G and empty pseudotyped particles. HEK293 T/ACE2 cells were infected with SARS-CoV-2-Spike, VSV-G or empty pseudotype lentiviruses at 0.5 TCID50 per cell. Seventy-two hours later, cells were stained for X-Gal and observed microscopically. Cell nuclei were counterstained with Nuclear Fast Red Solution. Scale bar = 20 µm.

FIG. 16. Dose-response curves of neutralization of SARS-CoV-2 Spike pseudotyped lentivirus infection by Sindbis vaccination. Luciferase-encoding SARS-CoV-2 pseudotyped particles were incubated with C57BL/6J mouse sera collected at (A) 21 and (B) 75 days post vaccination with Sindbis expressing Sars-CoV-2 spike (left panels), Sars-CoV-2 Spike in combination with αOX40 (middle panels) and αOX40 alone (right panels) compared to the Naive group. The data presented are the mean of n = 5 biological replicates with n = 2 technical replicates each curve.

FIG. 17. T cell activation and differentiation. Comparison of intraperitoneal and subcutaneous immunization routes with SV.Spike in combination with αOX40. Mice were immunized with SV.Spike via the intraperitoneal or intramuscular route in combination with αOX40. Naive mice were used as control. Spleens were excised and single- cell suspensions were stained for flow cytometry analysis on day 7 after prime doses. (A) Proliferation of CD4 T cells indicated by Ki67+ expression. (B) CD4 T cell activation indicated by CD44+ expression. (C) Th-1 type T cell differentiation indicated by double-positive ICOS+Tbet+ expression. Cytotoxic CD4+ (D) and (E) CD8 T Cells indicated by GrB+ expression. (F) CXCR5+ upregulation indicates Tfh cells differentiation. Bars or symbols represent means ± SEM (n=5 mice each group). Statistical significance was determined with the Kruskal-Wallis test followed by the he Dunns’ test. n.s. > 0.05, **p<0.005, * * *p≤ 0.001.

FIG. 18. T Cells of SV.Spike+αOX40 vaccinated mice show a unique transcriptional signature compared to single agents. Combination therapy markedly changes the transcriptome signature of T cells favoring T cell differentiation towards effector T cells shortly after prime vaccination. T cells were isolated and RNAseq was performed. Gene ontology analysis for biological processes was performed by STRING. Significantly upregulated DEGs (>2 fold) from T cells isolated from SV.Spike and/or αOX40 treated C57BL/6J mice compared to naive group were analyzed. Each bar represents a functional annotation (Strength ≥1). Percentage of contributing upregulated DEGs per GO term is indicated for αOX40 (top), SV.Spike (middle) and combination vaccinated group (bottom). Biological processes are further clustered for Apoptosis (light green), Cell Cycle (red), Cellular Signaling (blue), Chemokines/ Chemotaxis (orange), Cytokines (pink), Immune response (light blue) and Mitochondrial ATP Production (green).

FIG. 19. SV.Spike in combination with αOX40 drives cytotoxic T cell differentiation. C57BL/6J mice were prime/boost immunized with SV.Spike and/or αOX40. Naive mice were used as control. Spleens (A-D) and lungs (E-H) were excised and single-cell suspensions were stained for flow cytometry analysis on day 21 after prime doses. Cytotoxic CD4+ (A, B, E, F) and CD8+ T cells (C, D, G, H) were present as indicated by granzyme B+ positive cells in spleens and lungs. (n=5 mice per group). Representative blots are shown. Bars or symbols represent means ± SEM. Statistical significance was determined with the Kruskal-Wallis test followed by the he Dunns’ test. n.s. > 0.05, *p<0.05, **p<0.005, ***p≤ 0.001.

FIG. 20. SV.Spike in combination with αOX40 drives T cell activation. Mice were prime/boost immunized with SV.Spike and/or αOX40. Naive mice were used as control. Spleens (A-D) and lungs (E-H) were excised and single- cell suspensions were stained for flow cytometry analysis on day 21 after prime vaccine doses. Activated CD4+ (A, B, E, F) and CD8+ T cells (C, D, G, H) were present as indicated by CD44+ positive cells in spleens and lungs. Bars represent means and each symbol represent an individual mouse Statistical significance was determined with the Kruskal-Wallis test followed by the he Dunns’ test. (n=5 mice per group) n.s. > 0.05, *p<0.05, **p<0.005, * * *p≤ 0.001.

FIG. 21. Rechallenging immunized mice with spike antigen promotes a fast response of immune effector memory T cells. T cell activation was assessed in C57BL/6J vaccinated mice after rechallenge with Sindbis carrying SARS-CoV-2-Spike. Mice were rechallenged with SARS-Cov-2 spike on day 100 after prime vaccinations. Spleens were excised on day 103 and single cell suspensions were stained for flow cytometry analysis. (A) CD44+ positive CD4+ T cells and representative plots (B) indicating T cell activation shortly after rechallenge. (n=5 mice per group, or as otherwise indicated). Bars or symbols represent means ± SEM. Statistical significance was determined with the Kruskal-Wallis test followed by the he Dunns’ test. n.s. > 0.05, *p<0.05.

FIG. 22. Sindbis Replicon Vector expressing SARS-CoV2 spike protein. To prepare the replicon vector, the plasmid is digested with a restriction enzyme directly following the poly A site . The linear plasmid DNA provides a template for T7 polymerase mRNA transcription from the T7 promoter. Replicase, SV RNA polymerase ; Psg, subgenomic promoter for intracellular transcription; Spike sequence obtained from Biodefense and Emerging Infections Research Resources Repository (BEI Resources SARs Cov2 52310) Poly A, poly A tail transcribed onto spike mRNA; AmpR, ampicillin resistant gene; ColE, plasmid origin of replication. Numbers show nucleotide positions of genes in the replicon plasmid.

FIG. 23. Sindbis Replicon Vector expressing anti-OX40, IL12 and SARS-CoV2 Spike. The Plasmid was digested as in FIG. 22 for transcription from the T7 promoter. Descriptions as in FIG. 22 with added 2Psg, second subgenomic promoter; anti-OX40 heavy and light chains; T2A, peptide termination sequence; mouse IL12 gene.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein expressly and by reference, and every polynucleotide sequence referred to herein includes its complementary sequence, and its reverse complement. All segments of polynucleotides from 10 nucleotides to the entire length of the polynucleotides, inclusive, and including numbers and ranges of numbers there between are included. DNA sequences includes the RNA equivalents thereof to the extent an RNA sequence is not given. Every DNA and RNA sequence encoding polypeptides disclosed herein is encompassed by this disclosure, including but not limited to sequences encoding all recombinant proteins that comprise any complete SARS-CoV-2 protein, or an antigenic segment thereof, any antibody or antigen binding segment thereof, and any other protein encoded by the described modified viruses. All of the amino acid sequences and nucleotide sequences associated with any accession numbers are incorporated herein by reference as they exist in the database as of the date of the filing of this application or patent. The disclosure includes all polynucleotide and protein sequences described herein expressly or by reference that are between 80.0% and 99.9% identical to the described sequences. The proteins may comprise one or more than one amino acid change. Such changes can comprise conservative or non-conservative amino acid substitutions, insertions, and deletions. Any one or combination of components can be omitted from the claims, including any polynucleotide sequence, any amino acid sequence, and any one or combination of steps.

The disclosure includes all immune responses described below and in the Examples, including but not necessarily limited to antibody responses and T cell responses, and all combinations thereof. The disclosure provides for eliciting a synergistic immunological response. A synergistic response includes but is not necessarily limited to a synergistic effect on stimulation of T cells, antibodies, and a combination of T cells and antibodies, and on the transcriptome profile of T cells. A synergistic effect stimulates an improved immune response relative to use of a modified virus encoding a spike protein alone, or an immunomodulatory agent alone.

In embodiments, the disclosure provides a modified Alphavirus and pluralities of modified Alphavirus particles that are modified for use in stimulating an immune response against SARS-CoV-2. In embodiments, the modified Alphavirus encodes one or more SARS-CoV-2 proteins or antigenic segments thereof that are selected from the SARS-CoV-2 spike glycoprotein (S), envelope protein (E), membrane protein (M), and nucleocapsid protein (N). In embodiments, the SARS-CoV-2 protein comprises all or a segment of the viral spike receptor binding domain (RBD).

In embodiments, a modified Alphavirus of the disclosure encodes and expresses a spike protein that is expressed by the Wuhan-Hu-1 SARS-CoV2 virus, but the disclosure is not limited to this sequence and includes proteins and antigenic fragments thereof expressed by so-called SARS-CoV-2 variants, such variants including but not necessarily limited to variants currently referred to as variants of interest, variants of concern, and variants of high consequence. In embodiments, the described modified viruses encode spike protein or one or more antigenic fragments thereof from SARS-CoV-2 variants that include at least one of an L452R or E484K spike protein amino acid substitution. In embodiments, the spike protein or antigenic fragment of it is from SARS-CoV-2 variants currently referred to as B.1.1.7, B.1.351, P.1, P.2., B.1.427, B.1.429, or B.1.526.1. Any spike protein variant described herein may be compared to SEQ ID NO: 1 for reference to amino acid position.

In embodiments, an antigenic segment of a SARS-CoV-2 protein that is expressed by the described modified alphaviruses comprises or consists of the receptor binding domain (RBD) of the spike protein. In embodiments, the RBD comprises or consists of amino acids 333-527 of the spike protein. In embodiments, the antigenic segment of the spike protein comprises a receptor binding motif (RBF), which may include amino acids 438-506 of the spike protein.

The described modified alphaviruses may also encode and express one or more immunomodulating agents to generate effective anti-viral immune responses, including but not necessarily limited to T cell responses.

In embodiments, the described modified viruses are any type of alphavirus. In embodiments, the alphavirus that is modified according to the present disclosure comprises one or more modifications in the virus and/or plasmids that are used to make the modified viruses that are described in Current Opinion in Schlesinger and Dubensky, Biotechnology 1999,10:434-439, from which the description is incorporated herein by reference. In embodiments, the alphavirus of the disclosure comprises a modified Barmah Forest virus, Barmah Forest virus complex, Eastern equine encephalitis virus (EEEV), Eastern equine encephalitis virus complex, Middelburg virus, Middelburg virus complex, Ndumu virus, Ndumu virus complex, Semliki Forest virus, Semliki Forest virus complex, Bebaru virus, Chikungunya virus, Mayaro virus, Subtype Una virus, O′Nyong Nyong virus, Subtype Igbo-Ora virus, Ross River virus, Subtype Getah virus, Subtype Bebaru virus, Subtype Sagiyama virus, Subtype Me Tri virus, Venezuelan equine encephalitis virus (VEEV), VEEV complex, Cabassou virus, Everglades virus, Mosso das Pedras virus, Mucambo virus, Paramana virus, Pixuna virus, Western equine encephalitis virus (WEEV), Rio Negro virus, Trocara virus, Subtype Bijou Bridge virus, Western equine encephalitis virus complex, Aura virus, Babanki virus, Kyzylagach virus, Sindbis virus, Ockelbo virus, Whataroa virus, Buggy Creek virus, Fort Morgan virus, Highlands J virus, Eilat virus, Salmon pancreatic disease virus (SPDV), Southern elephant seal virus (SESV), Tai Forest virus, or Tonate virus. All alphavirus nucleotide and amino acid sequences, including all viral genomic sequences, expression vector and plasmid sequences, described in PCT publication WO/2021/007276 are incorporated herein by reference.

In embodiments, an immunomodulating agent that is encoded and expressed by a described modified virus, or is co-administered with a modified virus, comprises an antibody or antigen binding fragment thereof. In embodiments, the antibody or antigen binding fragment thereof has a receptor agonist function. In embodiments, the described immunomodulating agent comprises an anti-OX40 antibody. The amino acid sequences of suitable anti-OX40 antibodies are known in the art, and representative sequences are provided herein. In embodiments anti-OX40 antibody comprises complementarity determining regions (CDRs) and may include the complete heavy and light chain variable regions as described in PCT publication WO/2021/007276, from which all anti-OX40 antibody amino acid sequences and nucleotide sequences encoding them are incorporated herein by reference. The antibody may be of any isotype. In embodiments, the isotype is an IgG, which may be an IgG2a isotype.

In embodiments, the disclosure provides modified alphaviruses that co-express or are delivered in conjunction with an immunomodulating agent that may be distinct from an anti-OX40 antibody, representative examples of which include but are not limited to therapeutic proteins, such as cytokines, including but not necessarily limited to one or more interleukins (ILs). In an embodiment the IL is IL-12. In an embodiment, the IL is any IL described in PCT publication WO/2021/007276 from which the description of interleukins and their amino acid sequences is incorporated herein by reference.

In one embodiment, the present disclosure provides modified Sindbis virus (SV) vectors, which combine a SV-based approach of delivering the described SARS-CoV-2 proteins or antigenic segments thereof in combination with one or more immunomodulating agents. In this regard, SV is an RNA virus without replicative DNA intermediates and poses no risk of chromosomal integration or insertional mutagenesis. Hence, its presence within cells is transitory.

In non-limiting embodiments, the disclosure provides a therapeutically effective amount of one or more Sindbis viral vectors expressing a gene encoding a SARS-CoV-2 protein or antigenic segment of the protein, and (b) either independently or by expression of the viral vector an intact anti-OX40 monoclonal antibody, or an OX40-binding fragment thereof. In embodiments, the anti-OX40 binding fragment is any of OX40-binding (Fab) fragments, Fab′ fragments, (Fab′)2 fragments, Fd (N-terminal part of the heavy chain) fragments, Fv fragments (the two variable domains), dAb fragments, single domain fragments or single monomeric variable antibody domain (e.g., a nanobody), isolated CDR regions, single-chain variable fragment (scFv), and other antibody fragments or derivatives thereof, provided they bind with specificity to the Fc. In an embodiment, the antibody comprises an anti-SARS V_{H}H single chain antibody.

In embodiments, the disclosure provides compositions and methods in which the Sindbis genome is split into two plasmids, one providing the replicon and the other providing the helper. This vector system may be used, for example, to electroporate in vitro transcribed viral RNA into a susceptible cell line to produce replicative defective Sindbis virus for use as a viral vector, wherein the viral vector contains, as a genome, the replicase RNA, but lacks Sindbis structural genes. Thus, in certain approaches, the Sindbis viral vector may be replication defective by way of deleting certain genes that are required to maintain its infectivity.

In embodiments, the disclosure provides modified Sindbis viral vectors, viral particles, pharmaceutical compositions comprising the viral particles, and methods comprising administering modified Sindbis-derived particles to individuals in need thereof.

In embodiments, the Sindbis virus vector or virus particle comprises a polynucleotide that encodes one or multiple (e.g., two or more) epitopes of one or more SARS-CoV-2 proteins. In certain embodiments, more than one SARS-CoV-2 protein or antigenic fragment may be included in the modified Sindbis virus, wherein each protein or antigenic fragment is separated by, for example, an enzyme cleavage site, or by a self-cleaving amino sequence. While T2A sequences are used in the Examples, other suitable ribosome skipping sequences may be used, and include but are not limited to P2A, E2A and F2A, the sequences of which are known in the art. In embodiments, the disclosure provides modified viruses that encode contiguously or separately non-overlapping spike protein epitopes to thereby reduce or prevent the formation of escape mutants.

In embodiments the disclosure provides isolated polynucleotides. By “isolated polynucleotide” is meant a nucleic acid (e.g., a DNA or RNA molecule) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or a described virus; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

In embodiments the polynucleotides and/or viral particles produced using the described modified Sindbis or other alphavirus vectors are introduced into a subject as a component of a pharmaceutical composition. Suitable pharmaceutical compositions can be prepared by mixing one described modified alphaviruses described herein with a pharmaceutically acceptable additive, such as a pharmaceutically acceptable carrier, diluent or excipient, and suitable such components are well known in the art. Some examples of such carriers, diluents and excipients can be found in: Remington: The Science and Practice of Pharmacy 23rd edition (2020), the disclosure of which is incorporated herein by reference. In embodiments, the pharmaceutical formulation does not comprise a cell culture, or a cell culture media. In embodiments, the pharmaceutical formulation is free from any cell culture media. In embodiments, the pharmaceutical formulation is free from any mammalian cells or mammalian cell culture. In embodiments, the pharmaceutical formulation is free from Lipid inorganic nanoparticles (LIONs).

The described modified viruses and pharmaceutical formulations comprising them can be administered using any suitable route and method. In embodiments, the amount of agent includes an effective amount of the SARS-CoV-2 protein(s) and one or more immunomodulatory agents to achieve a desired result. The desired result can comprise a prophylactic effect, or a therapeutic effect. In embodiments, sufficient viral particles are introduced such that a cell mediated immune response is mounted against the one or more SARS-CoV-2 proteins, and wherein such cell mediated immune response, which may be accompanied by a humoral response, is therapeutic in an individual who is infected by SARS-CoV-2, and wherein the individual may or may not exhibit COVID-19 infection symptoms. Thus, in embodiments, a composition of the disclosure is administered to an individual who is infected with SARS-CoV-2, or is suspected of having a SARS-CoV-2 infection. In embodiments, the composition is administered to an individual who is at risk for contracting a SARS-CoV-2 infection. In embodiments, the individual is of an age wherein such risk is heightened, such as any individual over the age of 50 years. In embodiments, the individual has an underlying condition wherein the risk of developing severe symptoms of COVID-19 infection is increased, including but not necessarily limited to any respiratory condition.

In embodiments, an effective amount of a composition is administered to an individual. An effective amount means an amount of the described modified virus that will elicit the biological or medical response by a subject that is being sought by a medical doctor or other clinician. In embodiments, an effective amount means an amount sufficient to prevent, or reduce by at least about 30 percent, or by at least 50 percent, or by at least 90 percent, any sign or symptom of viral infection, e.g., any sign or symptom of COVID-19. In embodiments, fever is prevented or is less severe than if the presently described vaccine had not been administered. In embodiments, viral pneumonia is inhibited or prevented. In an embodiment, administration of a described vaccine prevents a SARS-CoV-2 infection in an individual who is exposed to SARS-CoV-2. In embodiments, an effective amount comprises 10⁶ - 10⁹ transducing units (TU)/mL. In embodiments, about 10⁷ TU are administered.

In embodiments, an effective amount is provided a single time and provides a therapeutic or prophylactic effect. In embodiments, a dose is administered at least one time, at least two times, at least three times, at least four times or at least five times. In embodiments, a prime-boost dosage approach is used. The described approaches may be sufficient to provide a durable immune response that is protective against SARS-CoV-2 infection, and further described herein.

Administration of formulations comprising the modified Sindbis or other alphavirus vectors and/or viral particles as described herein can be performed using any suitable route of administration, including but not limited to parenteral, intraperitoneal, and oral administration. Parenteral infusions include intramuscular, intravenous, intraarterial, intraperitoneal, and subcutaneous administration. In one embodiment, a composition comprising the described modified viruses is provided in a form suitable for inhalation, including but not necessarily limited to an aerosol. In embodiments, a composition of the disclosure is lyophilized and is suitable for reconstitution in a liquid or aerosolizable form. In embodiments, a composition of the disclosure is administered to the lungs and/or gastrointestinal track of an individual. The compositions can be administered to humans, and are also suitable for use in a veterinary context and accordingly can be given to non-human animals, including non-human mammals such as canines, felines, and equine animals. In embodiments the disclosure provides a pancornavirus vaccine. In embodiments, in addition for the described uses related to SARS-CoV-2 and its variants, the described compositions and methods can be used for prophylaxis or therapy for any infectious member of the virus family Coronaviridae. Non-limiting examples of such viruses include any Coronavirus that that causes any of severe acute respiratory syndrome (SARS), Middle East respiratory syndrome (MERS), and feline Coronavirus (FCoV) that can lead to the development of feline infectious peritonitis (FIP), and any variants thereof.

In embodiments, a described composition may further comprise, or may be administered concurrently or consecutively with any anti-viral compound or other agent, non-limiting examples of which include Lopinavir, Ritonavir, one or more protease inhibitors, nucleoside derivatives such as Romidepsin and Ribavirin, Favipiravir, interferons, and therapeutic and prophylactic antibodies, such as antibodies identified or derived from convalescent patient plasma. In embodiments, the described modified viruses potentiate the described anti-viral compounds or antibodies, and may result in a synergistic anti-viral effect.

In embodiments, viral particle preparations can be produced for use in pharmaceutical preparations by adapting previous approaches so that the modified SV vectors that express the SARS-CoV-2 protein or antigenic binding fragment thereof, optionally along with the anti-OX40 and/or IL component, are produced. In embodiments, plasmids encoding the replicon or helper RNA are linearized and transcribed in vitro.

In more detail, in certain embodiments, a replicon plasmid encoding the Sindbis replicase genes (nsP1-nsP4) and a helper plasmid, encoding the viral structural genes (capsid protein C, E1, E2, E3, and 6K), are transcribed in vitro. To limit viral replication in vivo, the replicon genes are separated from the structural genes, which additionally contain a mutated packaging signal to prevent incorporation into virus particles. Virus particles are produced by transient transfection of baby hamster kidney (BHK) cells with in vitro synthesized Sindbis replicon RNA and helper RNA transcripts. Within the cell, genomic RNA is replicated by the Sindbis replicase and expressed from the capped replicon RNA transcript. Structural proteins are expressed from the helper RNA transcript. Only the replicon RNA is packaged into the capsid to form the nucleocapsid, which then associates with the viral glycoproteins E1 and E2 and buds out of the cell. The resulting virions contain the capped SV single-stranded RNA message for nsP1-nsP4 genes, which encode the viral replicase, and may include a subgenomic promoter (Psg).

The viral particles can be purified using any suitable technique to any desired degree of purity, and combined with one or more pharmaceutically acceptable excipients, carriers and the like, as described above.

In certain embodiments, the SV vectors used in this disclosure are generated from the Sindbis strain AR339, the genomic and protein sequence of which is known are the art, and which does not cause disease in humans. In embodiments, the SV AR339 has the sequence as available under GenBank: MH212167.1, from which all of the amino acid and nucleotide sequences from which are incorporated herein by reference as of the effective filing date of this application or patent. Nonetheless, to limit even transient adverse effects, in embodiments, the presently provided vectors are attenuated as discussed above by splitting the SV genome and removing the packaging signal from the genomic strand that encodes the structural genes. Thus, the described vectors cannot propagate beyond the cells they initially infect.

In certain embodiments, to further increase safety, the disclosure provides for utilization of only the replicon strand to produce vaccine formulations. Accordingly, in certain embodiments, no SV structural protein coding sequences are present in the described vaccines.

In one non-limiting approach, the SV replicon strand is used to express the spike protein isolated from the plasmid NR-52310, encoding the SARS-CoV-2, Wuhan-Hu-1 spike glycoprotein gene. In a non-limiting approach, expression of the spike glycoprotein (gp) or a derivative thereof from the replicon is achieved by transfection into Baby Hamster Kidney (BHK) cells or other suitable mammalian cells, followed by collection of the supernatant within a suitable period of time, such as about 48 hours after transfection. Western blot and Elisa using a commercially available antibody, such as ProSci catalog numbers 3223 and 3525, or any other suitable antibody, can be used to confirm expression if desired. Representative and non-limiting examples of the described approaches are provided in the Examples, which demonstrate production of high affinity antibodies and metabolic reprogramming of T cells that is expected to confer long-term memory to one or more SARS-CoV-2 antigens.

In embodiments, use of the compositions and methods of this disclosure stimulates production of SARS-CoV-2 protective antibodies, which may include neutralizing antibodies. The term “neutralizing antibody” refers to an antibody or a plurality of antibodies that inhibits, reduces or completely prevents viral infection.

The disclosure provides for measuring antibody and T cell responses to the spike protein, and/or to any other SARS-CoV-2 proteins that are included in the described vaccines. Analysis of the antibody responses can be performed using any suitable control, such as the NR-52306 spike glycoprotein RBD recombinant protein from SARS-CoV-2, Wuhan-Hu-1 produced from HEK293T cells. In embodiments, this spike protein is as available under NCBI Reference Sequence: NC_045512.2, from which all amino acid and nucleotide sequences are incorporated herein by reference as they exist on the effective filing date of this application or patent.

To determine T cell responses, and as further illustrated by the Examples, the disclosure includes use of mouse models, such as BALB/c derived CT26-Luc-NR-52310 spike glycoprotein cells. The disclosure includes safety and pharmacokinetic analysis. For example, cytotoxicity can be determined in vitro by diminution of luciferase signal. The disclosure includes in vivo analysis, such as by injecting the described cells into BALB/c mice and measuring the ability of the elicited T cells to suppress their growth as compared to CT26-Luc cells. It is expected that positive results will be obtained, and any other assays that directly impact COVID-19 infection can be performed to advance human implementations, and may include virus pseudotypes to preclude safety concerns.

In embodiments, the disclosure provides kits and articles of manufacture. Either may contain one or more sealed containers that contain one or more plasmids for producing the described modified viruses. The kit or article of manufacture may comprise a form of the vaccine that is suitable for injection or oral delivery, including by inhalation. The kit or article of manufacture may further include cell culture media, and/or cells suitable for producing the described modified viruses. The kit or article of manufacture may include printed material, such as a label or product insert that provides an indication that the contents of the container is for use in prophylaxis and/or therapy for a SARS-CoV-2 infection.

A representative and non-limiting example of a DNA sequence that corresponds to the modified viruses as further described herein is provided in SEQ ID NO:5. Within SEQ ID NO:5, the following proteins are encoded: Anti-OX40 IgG2a Heavy Chain, nucleotides 7661-9085; Anti-OX40 IgG2a Light Chain, nucleotides 9270-9992; IL- 12, nucleotides 10056-11675; SARS CoV2 Spike protein, nucleotides 11751-15599. The plasmids or other expression vectors and the RNA transcribed from them can, as further described herein, include other features. For instance, in SEQ ID NO:5 includes a 2Psg sequence at nucleotides 9086-9209 and T2A coding sequences at nucleotides 9993-10055 and 11676-11740. The disclosure includes all RNA equivalents of SEQ ID NO:5 (e.g., where U is substituted for T).

A representative and non-limiting example of an Anti-OX40 IgG2a Heavy Chain protein sequence is provided as SEQ ID NO:2:

### Anti-OX40 IgG2a Heavy Chain

MGQSRYLLFLATLALLNHLSLAMAEVQLVESGGGLVQPGGSLRLSCAASG
FTFSNYTMNWVRQAPGKGLEWVSAISGSGGSTYYADSVKGRFTISRDNSK
NTLYLQMNSLRAEDTAVYYCAKDRYSQVHYALDYWGQGTLVTVAAKTTAP
SVYPLAPVCGDTTGSSVTLGCLVKGYFPEPVTLTWNSGSLSSGVHTFPAV
LQSDLYTLSSSVTVTSSTWPSQSITCNVAHPASSTKVDKKIEPRGLTIKP
CPPCKCPAPNLLGGPSVFIFPPKIKDVLMISLSPIVTCVVVDVSEDDPDV
QISWFVNNVEVHTAQTQTHREDYNSTLRVVSALPIQHQDWMSGKEFKCKV
NNKDLPAPIERTISKPKGSVRASQVYVLPPPEEEMTKKQVTLTCMVTDFM
PEDIYVEWTNNGKTELNYKNTEPVLDSDGSYFMYSKLRVEKKNWVERNSY
SCSVVHEGLHNHHTTKSFSRTPGK (SEQ ID NO:2)

A representative and non-limiting example of an Anti-OX40 IgG2a light chain protein sequence is provided as SEQ ID NO:3:

### Anti-OX40 IgG2a Light Chain

MGQSRYLLFLATLALLNHLSLADIQMTQSPDSLPVTPGEPASISCRSSQS
LLHSNGYNYLDWYLQKAGQSPQLLIYLGSNRASGVPDRFSGSGSGTDFTL
KISRVEAEDVGVYYCQQYYNHPTTFGQGTKLEIKRADAAPTVSIFPPSSE
QLTSGGASVVCFLNNFYPKDINVKWKIDGSERQNGVLNSWTDQDSKDSTY
SMSSTLTLTKDEYERHNSYTCEATHKTSTSPIVKSFNRNECGSGEGRGSL
LTCGDVEENPGP (SEQ ID NO:3)

A representative and non-limiting example of an IL-12 amino acid sequence is provided is SEQ ID NO:4:

### Il-12

MGQSR YLLFLATLALLNHLSLARVIPVSGP ARCLSQSRNLLKTTDDMV
KTAREKLKHYSCTAEDIDHEDITRDQTSTLKTCLPLELHKNESCLATRET
SSTTRGSCLPPQKTSLMMTLCLGSIYEDLKMYQTEFQAINAALQNHNHQQ
IILDKGMLVAIDELMQSLNHNGETLRQKPPVGEADPYRVKMKLCILLHAF
STRVVTINRVMGYLS SAVPGVGVPGVGGSMWELEKDVYVVEVDWTPDA
PGETVNLTCDTPEEDDITWTSDQRHGVIGSGKTLTITVKEFLDAGQYTCH
KGGETLSHSHLLLHKKENGIWSTEILKNFKNKTFLKCEAPNYSGRFTCSW
LVQRNMDLKFNIKSSSSPPDSRAVTCGMASLSAEKVTLDQRDYEKYSVSC
QEDVTCPTAEETLPIELALEARQQNKYENYSTSFFIRDIIKPDPPKNLQM
KPLKNSQVEVSWEYPDSWSTPHSYFSLKFFVRIQRKKEKMKETEEGCNQK
GAFLVEKTSTEVQCKGGNVCVQAQDRYYNSSCSKWACVPCRVRSGSGEGR
GSLLTCGDVEENPGP (SEQ IDNO:4)

A representative and non-limiting example of a SARS CoV2 Spike protein sequence is provided as SEQ ID NO: 1:

GNATMFLLTTKRTMFVFLVLLPLVSSQCVNLTTRTQLPPAYTNSFTRGVY
YPDKVFRSSVLHSTQDLFLPFFSNVTWFHAIHVSGTNGTKRFDNPVLPFN
DGVYFASTEKSNIIRGWIFGTTLDSKTQSLLIVNNATNVVIKVCEFQFCN
DPFLGVYYHKNNKSWMESEFRVYSSANNCTFEYVSQPFLMDLEGKQGNFK
NLREFVFKNIDGYFKIYSKHTPINLVRDLPQGFSALEPLVDLPIGINITR
FQTLLALHRSYLTPGDSSSGWTAGAAAYYVGYLQPRTFLLKYNENGTITD
AVDCALDPLSETKCTLKSFTVEKGIYQTSNFRVQPTESIVRFPNITNLCP
FGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWN
SNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNK
CVNFNFNGLTGTGVLTESNKKFLPFQQFGRDIADTTDAVRDPQTLEILDI
TPCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARSV
ASQSIIAYTMSLGAENSVAYSNNSIAIPTNFTISVTTEILPVSMTKTSVD
CTMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGWT
FGAGAALQIPFAMQMAYRFNGIGVTQNVLYENQKLIANQFNSAIGKIQDS
LSSTASALGKLQDVVNQNAQALNTLVKQLSSNFGAISSVLNDILSRLDKV
EAEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGKA
HFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLN
EVAKNLNESLIDLQELGKYEQYIKWPWYIWLGFIAGLIAIVMVTIMLCCM
TSCCSCLKGCCSCGSCCKFDEDDSEPVLKGVKLHYT (SEQ ID NO:1)

The following examples are intended to illustrate but not limit the disclosure.

### Example 1

This Example demonstrates a Sindbis alphavirus vector (SV), transiently expressing the SARS-CoV-2 spike protein (SV.Spike), combined with the OX40 immunostimulatory antibody (αOX40) as a novel, highly effective vaccine approach.

Construction and characterization of Sindbis carrying the SARS-CoV-2-spike.

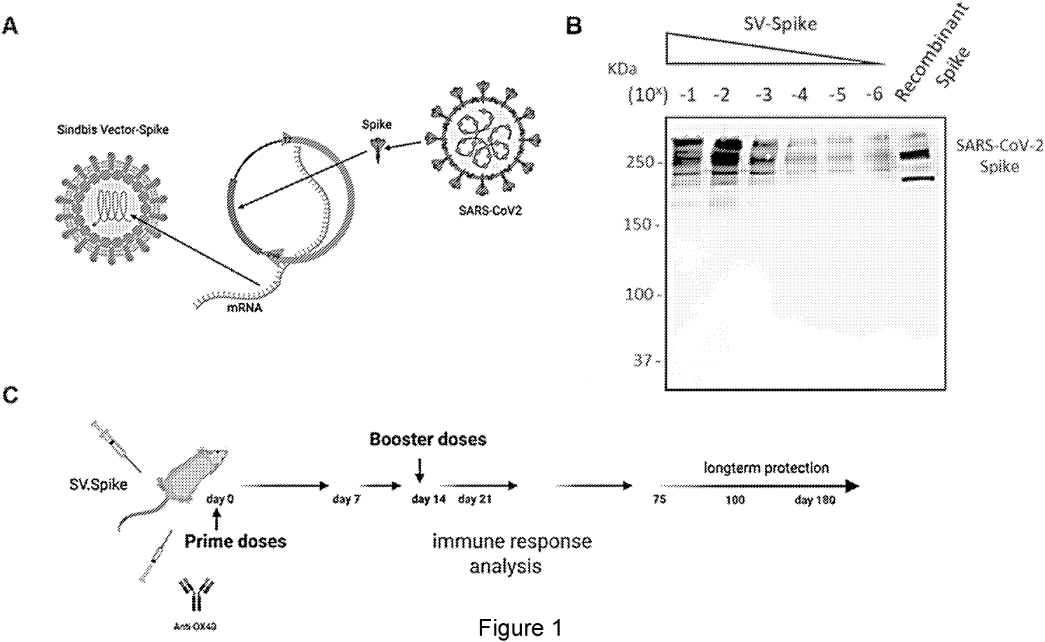

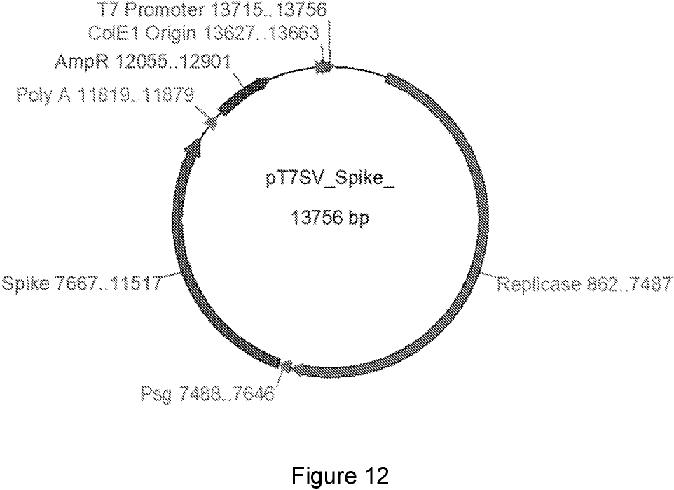

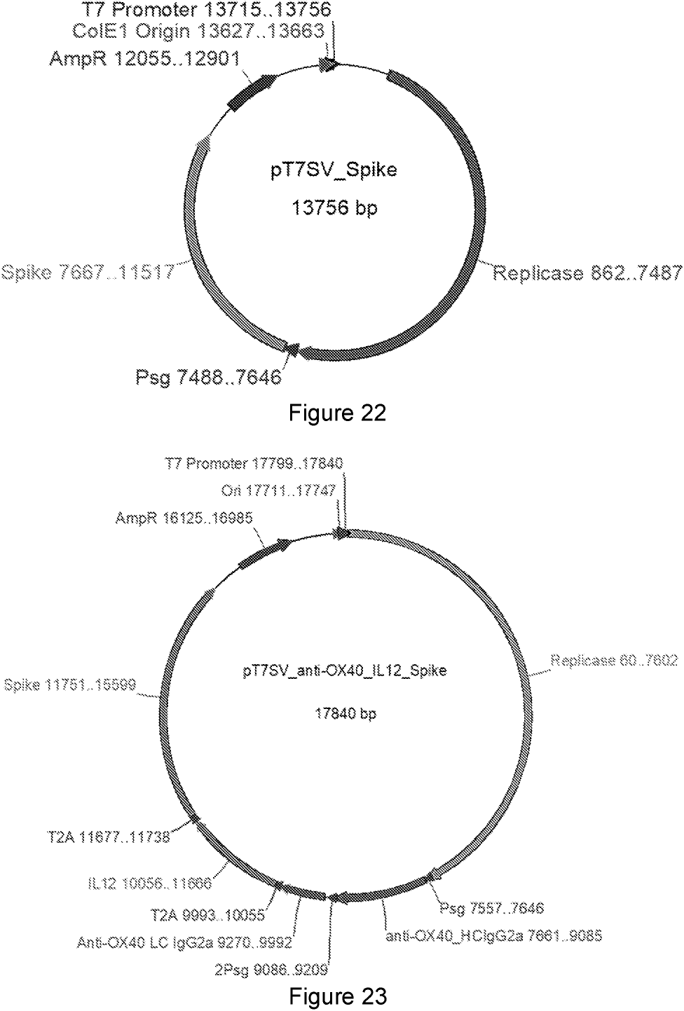

We designed and generated a Sindbis alphavirus replicon carrying the SARS-CoV-2 spike mRNA. SV vectors are generated from two plasmids: a replicon and helper (FIG. 1 and FIG. 12). Genes of interest (GOI) can be substituted for the 5kb structural genes that were removed to generate the helper plasmid. The plasmid encoding the structural genes does not contain a packaging signal, preventing further virus assembly beyond the initial preparation of the vectors in BHK-21 cells. Plasmids are transcribed from the T7 promoter and the RNA transcripts are electroporated into BHK-21 cells to produce viral vectors. Representative examples of plasmids used in this disclosure are shown in FIGS. 22 and 23.

The combination of SV vectors encoding a selected antigen with immunomodulatory antibodies makes them far more effective than they are alone[40; 41; 42]. In particular we have found that combining SV vectors expressing specific antigens with αOX40 generates very potent immune responses capable of eradicating tumors in multiple murine models and conferring long-term protection against tumor recurrences or rechallenges[40].

The overall design in the production of Sindbis SARS-CoV-2 Spike (SV.Spike) is illustrated in FIG. 1 and FIG. 12. We determined the expression of the full-length SARS-CoV-2 Spike from infected cells by western blot in FIG. 1B.

The immune responses induced by the Sindbis SARS-CoV-2 Spike (SV.Spike) vaccine candidate were analyzed in C57BL/6J mice. Groups of mice (n = 5) were immunized by intraperitoneal (i.p.) route, by prime-boost vaccine strategy with SV.Spike and/or αOX40, with 14 days difference between the two doses (FIG. 1C). Activation and priming of T cells were analyzed by flow cytometry and ELISPOT at day 7, 21 post-immunization (p.i.), while cytotoxic assay and transcriptomic analysis was performed in T cells isolated at day 7 p.i.. Metabolic activation of T and B cells was tested by Seahorse measurements (Agilent, CA) at day 7 and 21, respectively. Long-term memory T cell analysis was carried out at day 100 p.i.. The overall antibody responses were measured at all the indicated time points (from day 7 to day 100 p.i.; FIG. 1C).

Sindbis vaccine-elicited antibodies to SARS-CoV-2 spike.

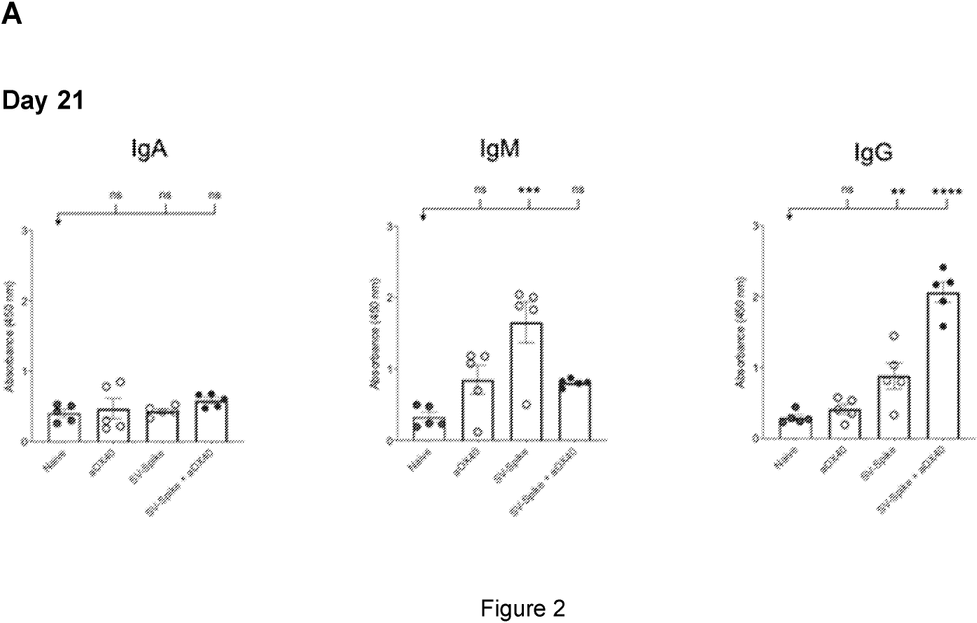

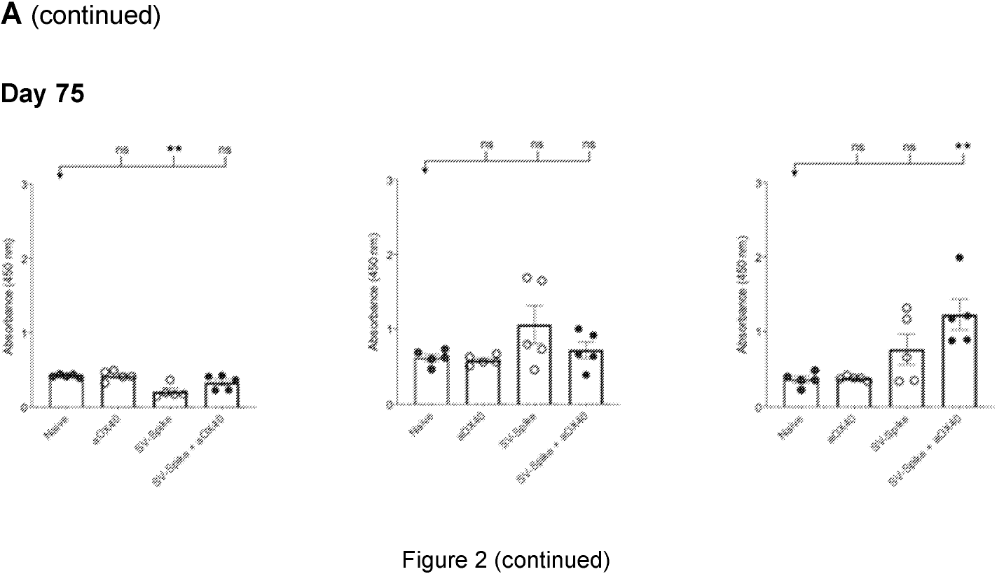

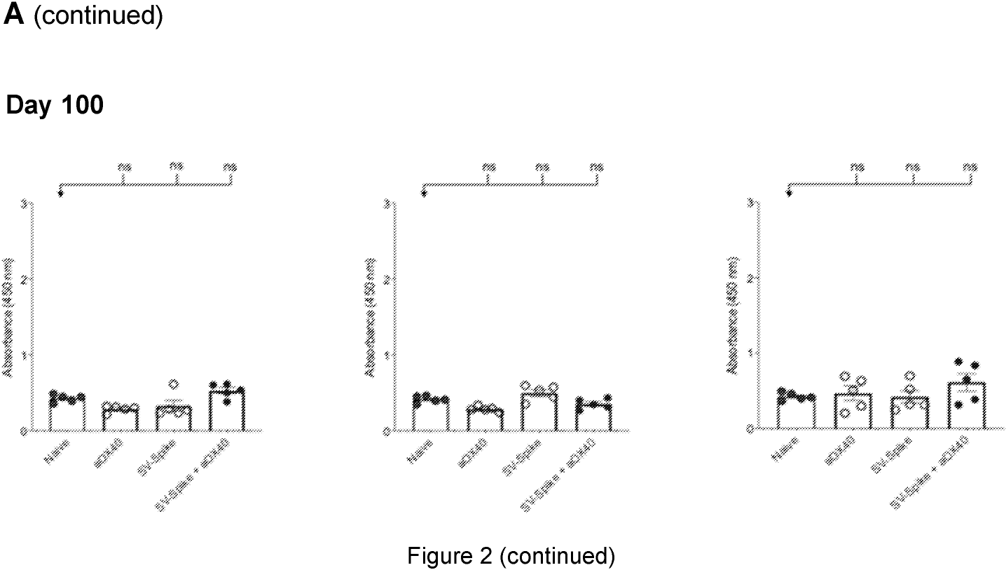

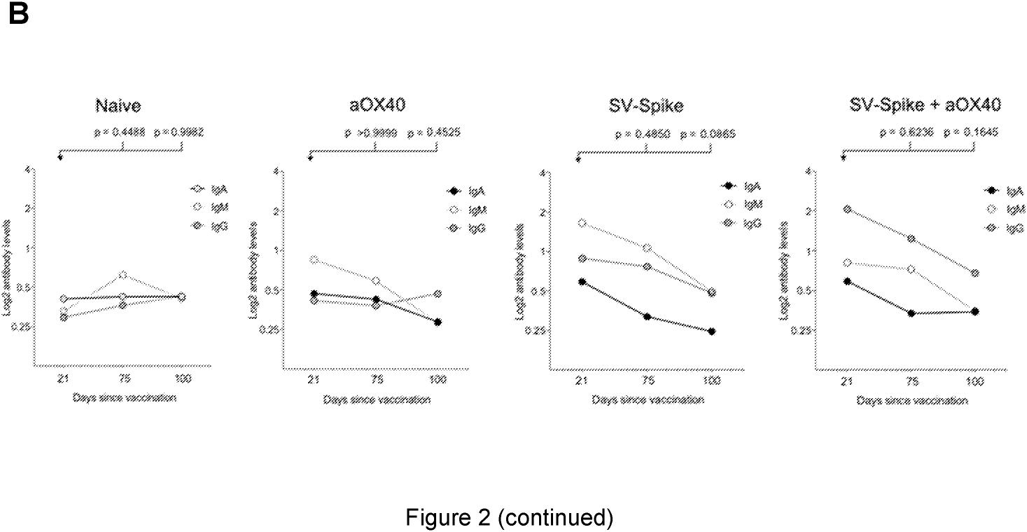

Serum IgM, IgG and IgA responses to SV.Spike, SV.Spike+αOX40, injections were measured on days 21, 75 and 100 days after vaccination by enzyme-linked immunosorbent assay (ELISA) against recombinant SARS CoV2 spike protein[3; 4]. Sera from all of mice tested showed reactivity to recombinant SARS-CoV-2 spike protein and, as might be expected, levels of antibodies varied based on the experimental group and time point. Consistent with previous reports[43; 44; 45], levels of IgM and IgG measured at day 21 and 75 post injection (p.i.) were significantly higher in the mice vaccinated with SV.Spike and combination of SV.Spike+□OX40 than in the mice who had received □OX40 alone or the naive group (FIG. 2A). Moreover, the SV.Spike+□OX40 group showed higher titers of IgG compared with only SV.Spike treatment, for which IgM was the predominant isotype and did not show seroconversion to IgG over the different time points. Specifically, both SARS CoV2-specific IgG and IgM antibodies demonstrated the highest expression on day 21 post immunization for the indicated groups (IgG-OD450 of 2.3 for SV.Spike+□OX40 serum, and IgM-OD450 of 1.9 for SV.Spike serum). At days 75 p.i., IgG were still significantly predominant in the sera of the mice immunized with the SV.Spike+□OX40 combination (IgG-OD450 = 1.3), whereas IgM reactivity did not significantly vary from day 21 to day 100 compared with the control groups (FIG. 2B). Instead, IgM levels in the SV.Spike mice showed a more significant decrease and less lasting reactivity from days 21 to 75 days p.i. (IgM-OD450 of 1.2) compared to the control group, whereas the IgG trend demonstrated significant high reactivity only at day 21 p.i.. Conversely, IgA levels did not show any significant difference in any of the groups and time points tested (FIGS. 2A, B). These data support the evidence that immunization of mice with SV.Spike combined with αOX40 elicits a strong and specific immune response, which is predominantly represented by SARS-CoV-2 IgG- specific antibodies.

Anti-SARS-CoV-2 spike neutralizing antibodies induced in Sindbis vaccinated mice block the SARS-CoV-2 spike protein from binding to hACE2 receptor proteins.

Immediately after SARS-CoV-2 was identified as the causative agent of the COVID-19 outbreak, it was shown that human ACE2 (hACE2) is the main functional receptor for viral entry[46]. We hypothesized that the virus-receptor binding can be mimicked in vitro via a protein-protein interaction using purified recombinant hACE2 and the Spike of the SARS-CoV-2 protein. This interaction can be blocked by virus naturalizing antibodies (NAbs) present in the test serum of vaccinated mice.

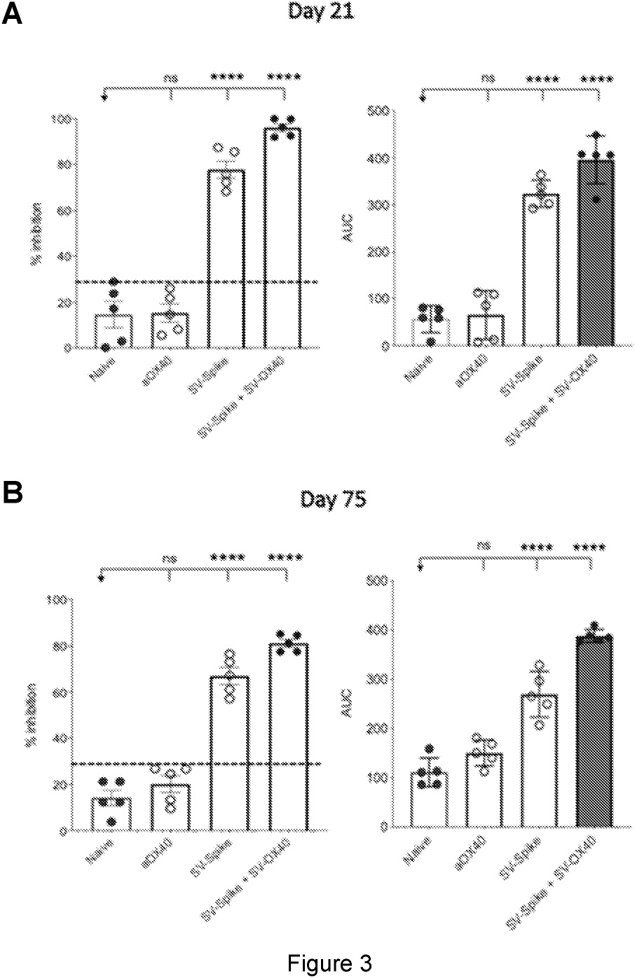

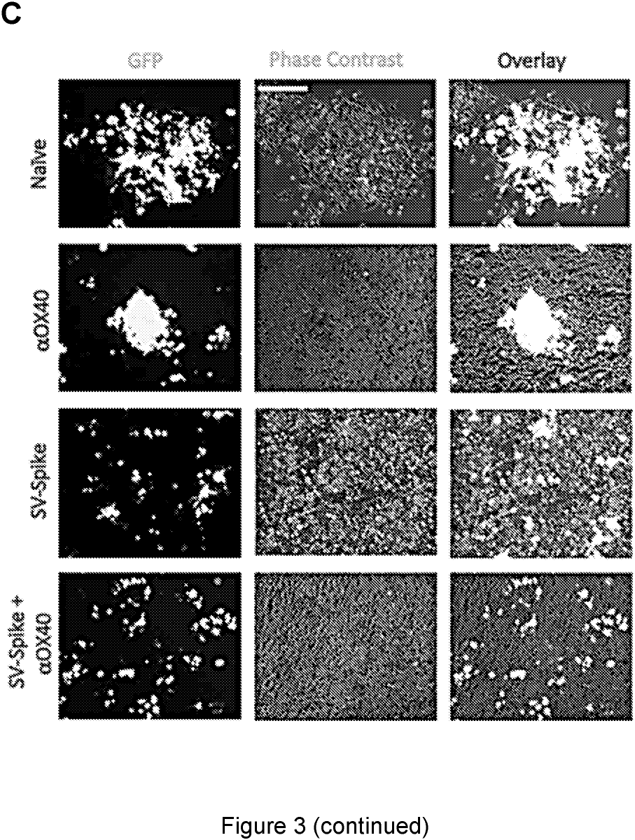

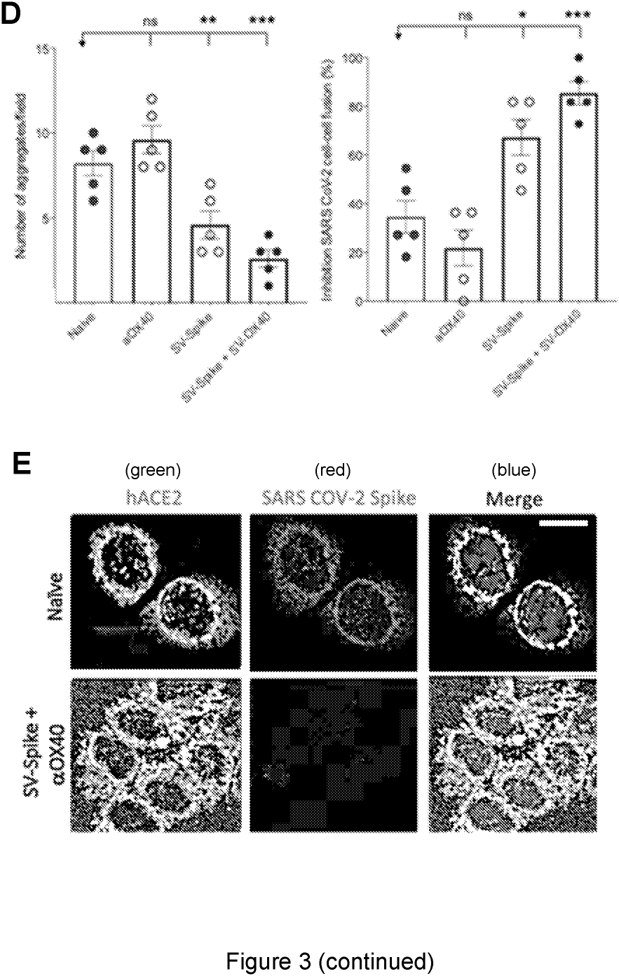

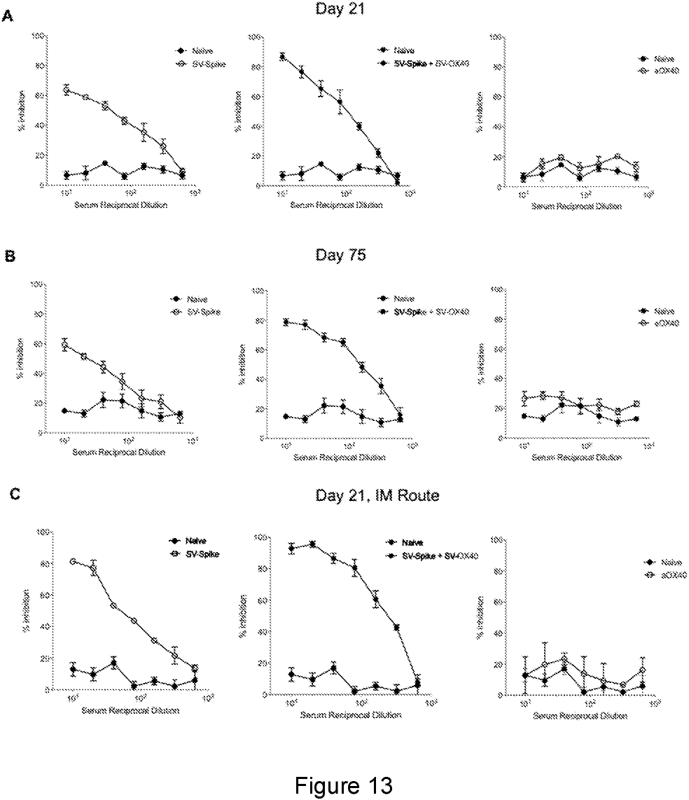

A competition ELISA assay was developed to detect whether SARS-CoV-2 Spike-specific antisera from mice immunized with αOX40, SV.Spike and SV.Spike+αOX40 could block the interaction between SARS-CoV-2 Spike and hACE2. Our assay demonstrated that the specific Spike-hACE2 binding can be neutralized by SV.Spike or SV.Spike+αOX40 sera in a dose-dependent manner, but not by sera from αOX40 alone or naïve groups (FIGS. 13A, B). Similar results are obtained by the intramuscular route (FIG. 13C). As shown in FIG. 3A, antibodies in the antisera from mice immunized with SV.Spike and combination of SV.Spike and αOX40 at day 21 post-immunization significantly inhibited the binding of SARS-CoV-2 Spike to hACE2 compared to the sera from naive mice, indicating that SV.Spike-induced antibodies could strongly neutralize SARS-CoV-2 infection by blocking the binding of Spike protein on the surface of SARS-CoV-2 to hACE2.

To investigate whether the neutralizing antibody response in immunized mice could maintain a high level for a longer period of time, we tested the neutralization activity of mice sera at 75 days post-immunization. The results showed that, although the overall antibody neutralizing capacity decreased compared to day 21, antibodies from SV.Spike and SV.Spike+αOX40 groups still significantly competed for the binding of the SARS-CoV-2 spike and hACE2 (FIG. 3B), indicating that our SV.Spike vaccine is able to induce relatively long-term neutralizing antibody responses.

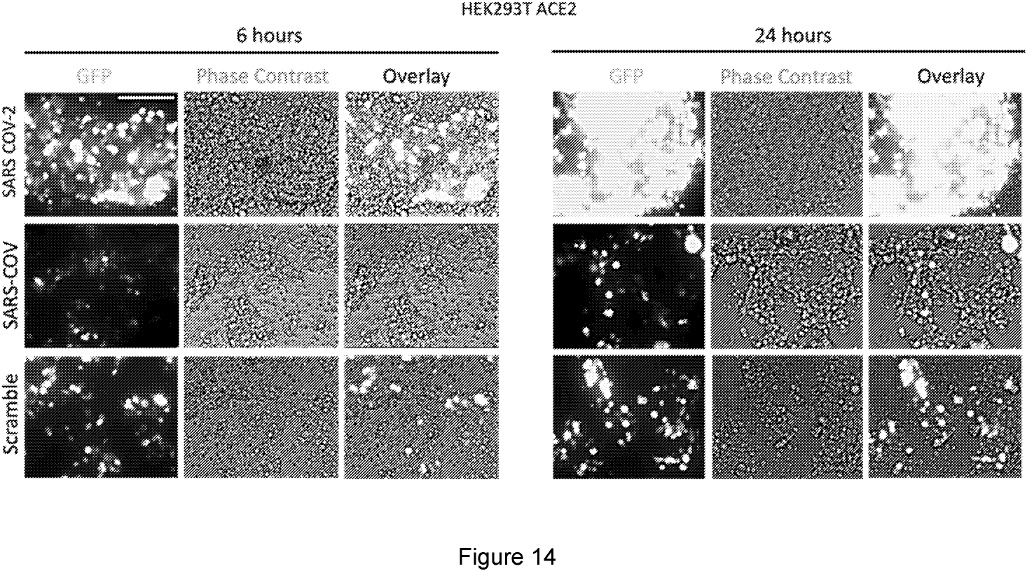

Next, we investigated if the serum from mice immunized with SV.Spike could inhibit the cell membrane fusion process for viral entry[47; 48; 49]occurring upon the binding of SARS-CoV-2 Spike Receptor Binding Domain (RBD) fragment to the ACE2 receptor on target cells. To establish an assay for measuring SARS-CoV-2-Spike-mediated cell-cell fusion, we employed HEK293T cells (a highly transfectable derivative of human embryonic kidney 293 cells, that contain the SV40 T-antigen) expressing both SARS-CoV-2 Spike and enhanced green fluorescent protein (EGFP) as effector cells and 293T cells stably expressing the human ACE2 receptor (ACE2/293T) as target cells. Notably, when the effector cells and the target cells were co-cultured at 37° C. for 6 h and 24 h, the two types of cells started to fuse at 6 h, exhibiting a much larger size and multiple nuclei compared to the unfused cells. These changes were more significant at 24 h, resulting in hundreds of cells fused as one large syncytium with multiple nuclei that could be easily seen under both light and fluorescence microscopy (FIG. 14). The cell fusions were observed in the cells transfected with SARS-CoV-2 Spike but not SARS-CoV Spike, whereas those cells transfected with EGFP only did not elicit such an effect, confirming that CoV-2 Spike-ACE2 engagement is essential for viral fusion and entry.

To determine whether the serum of mice immunized with SV.Spike can block Spike protein-mediated cell-cell fusion, we incubated the effector cells with serum from Naive, SV.Spike and/or αOX40 mice (diluted 1:100) at 37° C. for 1 h and then we co-cultured them with the ACE2/293T target cells. We found that not only were fewer fusing cells observed, but also the size of fused cells were visually smaller in the groups of SARS-CoV-2-Spike/293T effector cells pre-incubated SV.Spike with or without αOX40 sera compared to controls (FIG. 3C). Quantification of fused cells per field in at least four randomly selected fields revealed a remarkably lower number of cell-cell fusions in both SV.Spike and SV.Spike+αOX40 groups compared to all the other groups. Moreover, SARS-CoV-2 Spike-mediated cell-cell fusions were significantly inhibited by serum derived from SV.Spike+αOX40 vaccinated mice, indicating that addition of αOX40 to the vaccination protocol elicits antibodies with enhanced interference of syncytium formation mediated by SARS-CoV-2 infection (FIGS. 3C, D).

The interference of immunized sera NAbs with SARS-CoV-2-hACE2 binding was also determined by immunofluorescence experiments performed by culturing ACE2/293T cells with recombinant SARS-CoV-2 Spike previously incubated with serum from naive and SV.Spike and αOX40 immunized mice. The binding between Spike and hACE2 expressed on the cell surface was subsequently visualized via confocal fluorescence microscopy (FIG. 3E). As expected, Spike incubated with SV.Spike+αOX40 serum was incapable of binding to hACE2, while the control group showed evident co-localization with hACE2 on the cell surface.

Taken together, these data demonstrate that SV.Spike alone, and to a greater extent SV.Spike+αOX40 sera, can neutralize SARS-CoV-2 Spike-hACE2 interaction and in turn counteract virus entry mediated by cell-membrane fusion.

### Example 2

This Example demonstrates that SV.Spike vaccine prevents infection of SARS-CoV-2 in hACE2 transgenic mice.

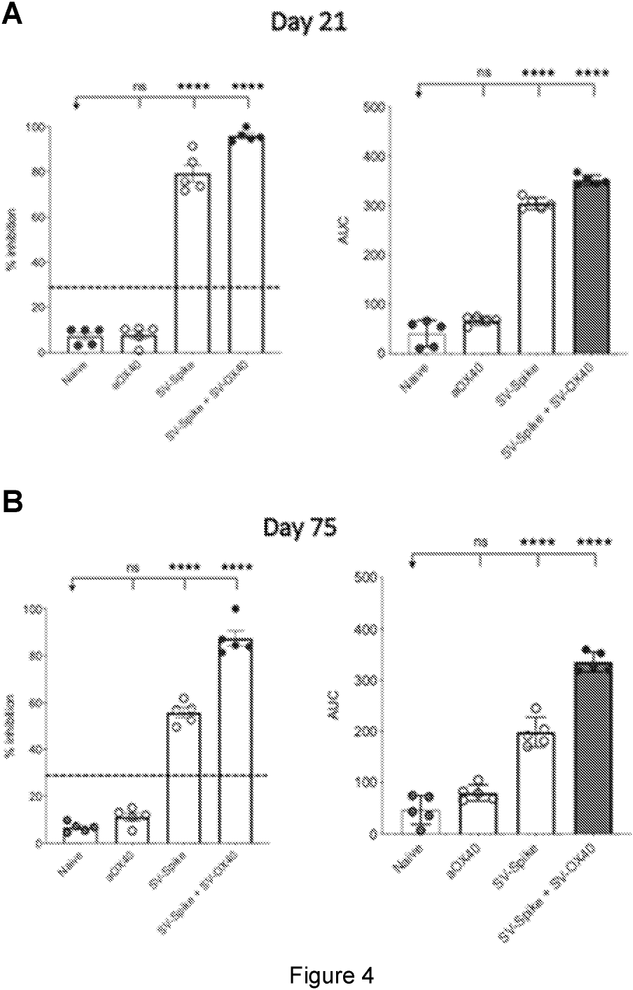

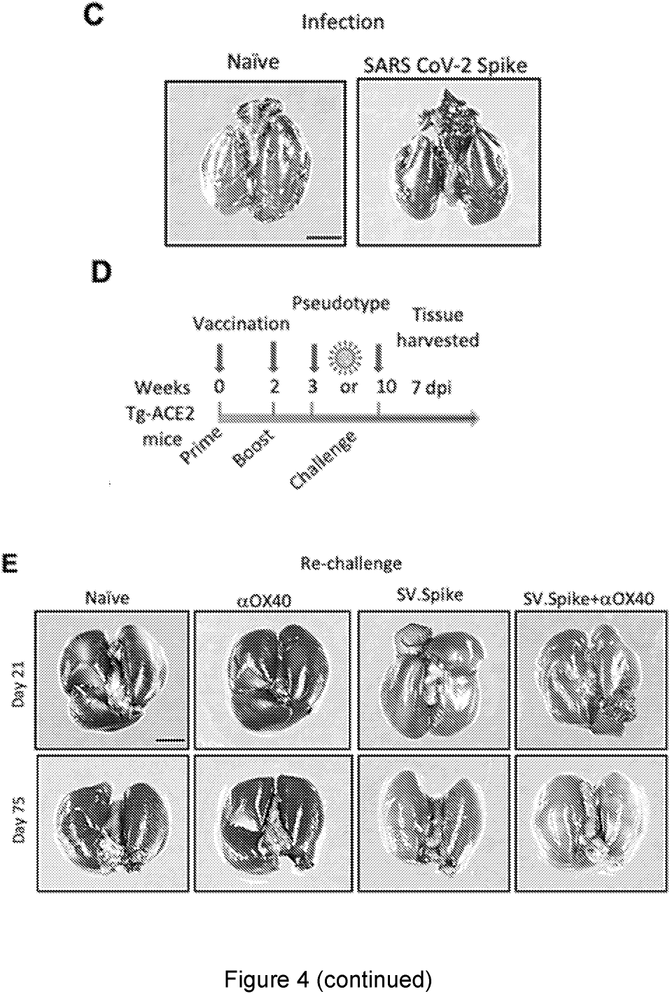

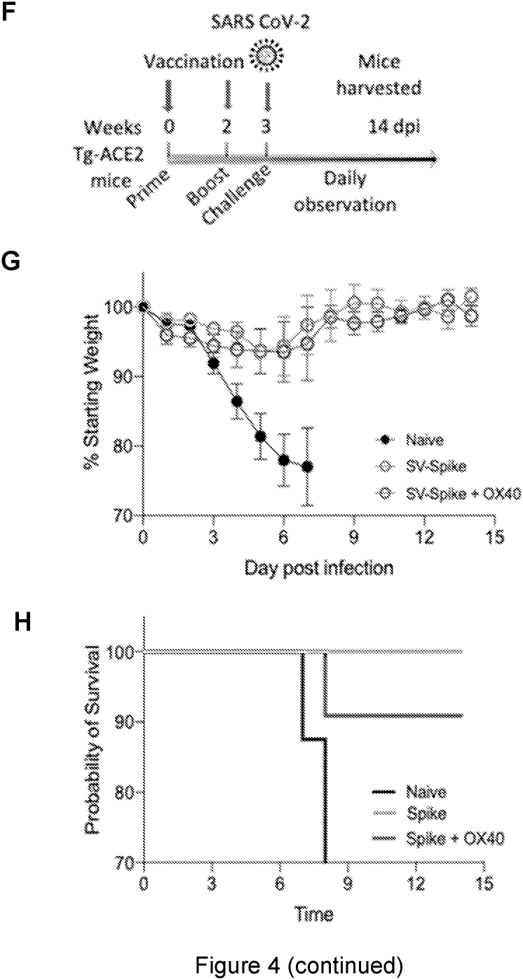

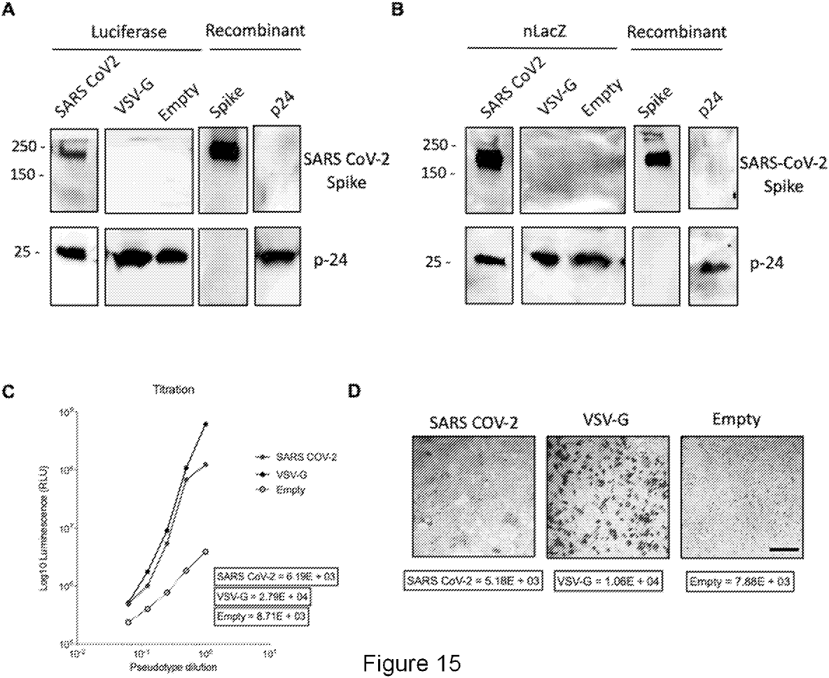

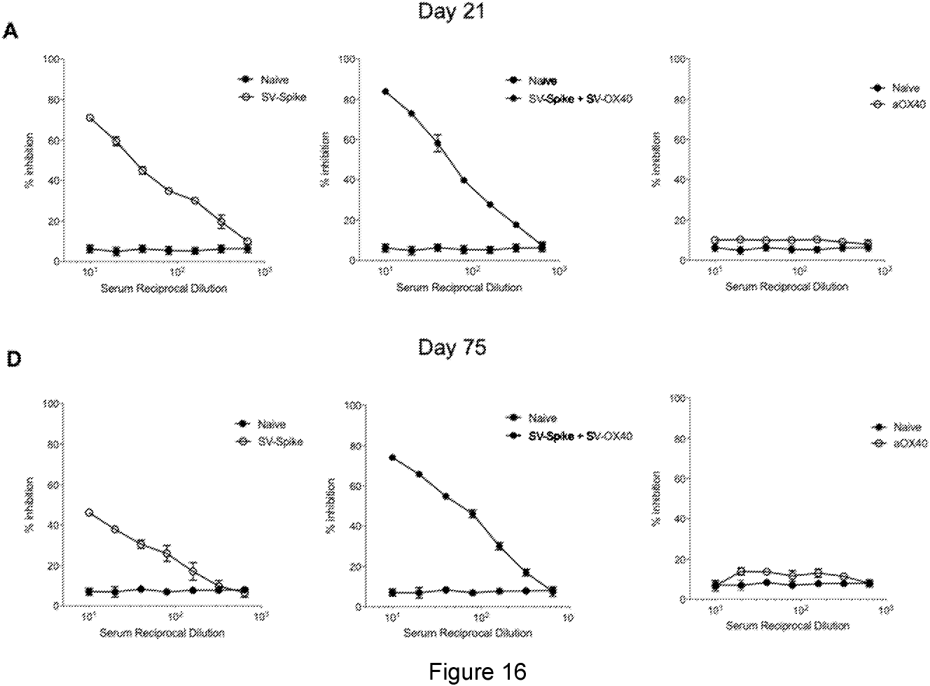

The neutralizing activity of serum from vaccinated mice was determined using Luciferase-encoding SARS-CoV-2 Spike pseudotyped lentivirus[50; 51] [52] (FIGS. 15A, C), by testing the impact of the serum on the lentivirus transduction. Serial dilutions (1:300, 1:600, 1:900: 1:1800, 1:3200 and 1:6400) of mice sera harvested at day 21 and 75 p.i. were incubated with equal amounts of lentivirus for 1 hour at 37° C., then plated on ACE2/293T cells. We then measured the amount of blocked pseudotyped viral particles in infected cells by determining the amount of luminescence reduction, which reflects the level of neutralizing antibody or molecular inhibitors in the sample. The results showed that the antisera could inhibit SARS-CoV-2 pseudotype infection in a dose-dependent manner (FIG. 16), consistent with the result from the antibody neutralization assay (FIG. 14). Our results demonstrate that sera from SV.Spike with or without αOX40 immunized mice groups resulted in significantly high levels of neutralizing antibodies both at day 21 and 75, since they overcame the pseudotyped lentivirus infectivity inhibition threshold of 30% (FIGS. 4A, B). Moreover, serum from these mice receiving combination of SV.Spike and αOX40 gave the highest levels of neutralization at day 21 after vaccination (95.3% of inhibition), with a slight decrease at day 75 (79% of inhibition). Naive and αOX40 groups did not develop a neutralizing antibody response (% inhibition < 30%) at the timepoints tested, consistent with their lack of SARS-CoV-2 Spike binding antibodies.

Recently, hACE2 transgenic (B6(Cg)-Tg(K18-ACE2)2Prlmn/J or hACE2-Tg) mice were used for the development of an animal model of SARS-CoV-2 infection[53]. In order to test pseudotyped lentivirus infectivity rate in vivo, we produced a nLacZ-encoding lentivirus expressing SARS-CoV-2 spike protein (FIGS. 15B, D) and we evaluated the vector expression following delivery to hACE-Tg mice airways, by administrating a single dose of nLacZ-pseudotype to 4-week-old hACE2-Tg mice by intranasal inhalation. After 7 days, the airways were harvested and intact glutaraldehyde-fixed tissues were processed for staining with X-Gal for detection of β-galactosidase activity expressed from the nuclear-localized lacZ reporter gene (nlacZ; FIG. 4C). Positive X-Gal staining observed in airways upon lentivirus intranasal administration indicated the successful SARS-CoV-2-Spike lentiviral vector expression and pseudotype delivery in mice airways.

In order to investigate the protective effects of SV.Spike vaccination in vivo, we subsequently immunized hACE2-Tg mice with the same strategy as used for the C57BL/6J mice (FIG. 1D). The hACE2-Tg mice were vaccinated at 0 and 2 weeks and then challenged with pseudotyped SARS-CoV-2 intranasally at day 21 and 75 post-immunization (FIG. 4D). The lungs were collected at 7 days post-challenge and pseudotype delivery was tested by X-Gal staining. As shown in FIG. 4E, the nLacZ-SARS-CoV-2-Spike lentivirus could not be detected in the lungs from SV.Spike+αOX40 immunized mice, while substantially reduced infectious virus burden was still detected in the lungs from SV.Spike treated mice compared with the naive group at the indicated time points. As expected, lungs from animals treated with αOX40 showed high amount of pseudotype particles, as indicated from the very high signal of X-Gal staining (FIG. 4E). Finally, protective immunity was also assessed in young adult vaccinated Tg-ACE2 mice challenged with live SARS-CoV-2 coronavirus. Three weeks after prime and boost vaccination doses, all mice were challenged with 10⁴ particles of SARS-CoV-2 via the intranasal (i.n.) route (FIG. 4F). We recorded the daily the body weight of each mouse after infection for a total of 14 days and found that the body weights of both SV.Spike and SV.Spike+αOX40 mice showed a slow decrease at 3-5 days post infection (dpi), with a progressive stabilization and increase of their weight at day 8-9 post infection. The naive group showed a faster decrease during 3-5 dpi (FIG. 4G), which led to early mortality around day 8 dpi (FIG. 4H). Vaccinated mice did not evidence any signs of disease at the time the experiment was terminated but were culled on day 14 as required by the protocol, which was performed in an ABSL3 facility. Together, these data suggest that combination of SV.Spike and αOX40 vaccine in mice conferred remarkably long-term protection against SARS-CoV-2 infection by eliciting a durable humoral response in mice.

### Example 3

This Example demonstrates that SV.Spike in combination with αOX40 metabolically reprograms and activates T cells shortly after prime vaccine doses.

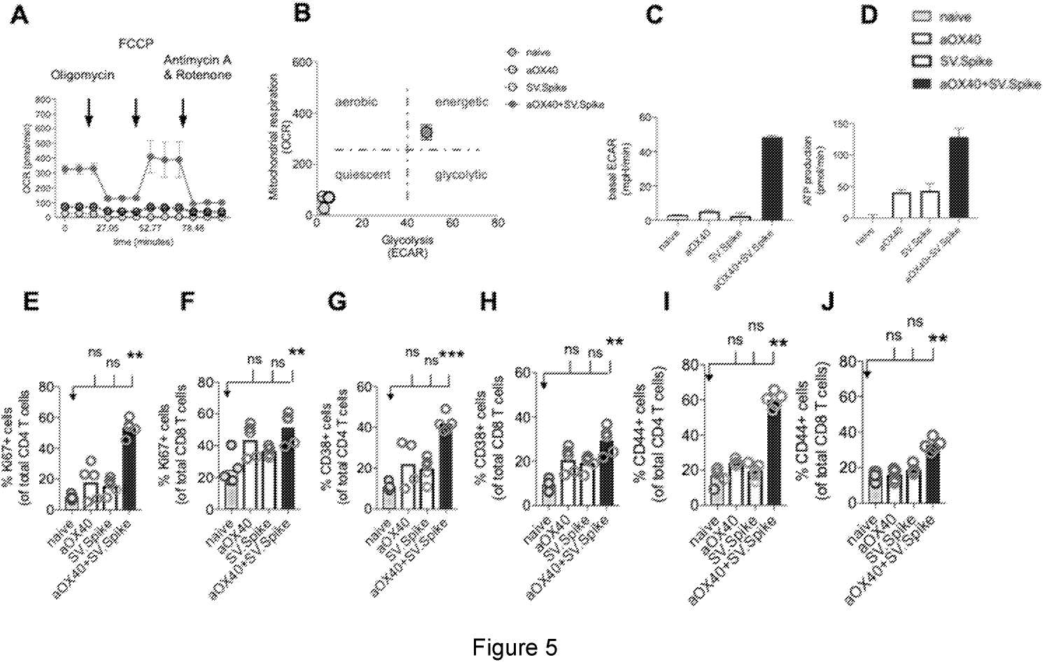

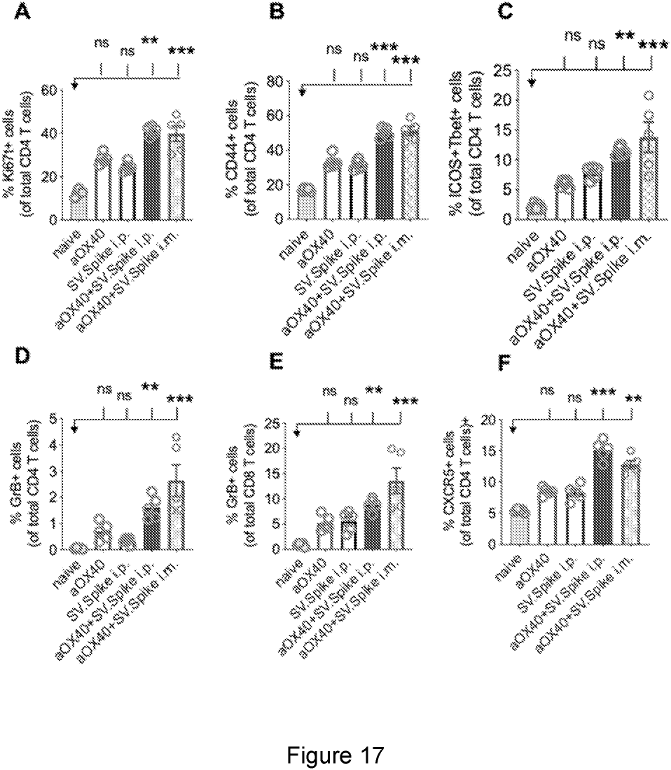

Analysis of SARS-COV-2 specific adaptive immune responses during acute COVID-19 identified coordination between SARS-COV-2-specific CD4+ T cells and CD8+ T cells in limiting disease severity[54]. We analyzed vaccine elicited T cell responses in the spleen 7 days after mice received prime doses of SV.Spike and/or αOX40 and compared the initial T cell response to naïve mice (FIG. 5). Similar results are obtained by the intramuscular route (FIG. 17). Spleens of mice were excised and a single cell suspension was stained and analyzed by flow cytometry.

For a successful vaccine-elicited immune response, differentiation of virus-specific T cells from the naive to the effector state requires a change in the metabolic pathways utilized for energy production[55]. Therefore, metabolic profiles of vaccine-induced T cells are of interest and correlate to vaccine-mediated immunity[56].

We performed metabolic analysis of isolated T cells from spleens in an Extracellular Flux Analyzer XFe24 (Seahorse Bioscience) to investigate metabolic changes of T cells. We found, that combining our SV.Spike vaccine with agonistic αOX40 antibody metabolically rewires T cells in vivo shortly after initial vaccine doses (FIGS. 5A-D). T cells freshly isolated from mice on day 7 after first doses with SV.Spike+αOX40 combination displayed a metabolic shift to a highly bioenergetic state compared to single agent treatment or naive mice that show a quiescent metabolism (FIGS. 5A-B). Naive T cells are quiescent and characterized by a metabolic program that favors energy production over biosynthesis. Upon T cell receptor (TCR)-mediated stimulation, T cells become activated and metabolically reprogrammed. The bioenergetic state of metabolically reprogrammed T cells is characterized by a strong increase of oxygen consumption rate (OCR), which is a parameter for mitochondrial respiration (FIG. 5A), and a strong increase of baseline extracellular acidification rate (ECAR) (FIG. 5C), which is measured as a parameter for glycolysis. It has been shown that TCR signaling is directly tied to glycolysis[57]. We found that T cells isolated from mice vaccinated with SV.Spike+αOX40 displayed a 3-fold increase of OCR and a 10-fold increase of ECAR compared to naive and single agent vaccinated mice. T cells switched to the energetic state ramped up their ATP production (FIG. 5D). A metabolic rapid adaptation is further required for effector T cells cytokine production and signaling. Rapid switch to type-1 cytokine production, such as IFNγ and granzyme B (GrB) in antiviral CD8+ T cells is more reliant on oxidative phosphorylation[58]. Indeed, immunophenotyping of CD4+ and CD8+ T cells by flow cytometry revealed rapid clonal expansion of CD4+ T and CD8+ T subsets within one week after prime vaccine doses indicated by Ki67 expression on gated CD4+ and CD8+ T cells. CD4+ T cells showed the highest expansion increase by 10-fold in the combination vaccinated group compared to naive and SV.Spike and αOX40 single agent immunized mice (FIGS. 5E-F). Both T cell subsets were highly activated, indicated by CD38 and CD44 expression (FIGS. 5G-J) underlining successful vaccine elicited effector T cell engagement by our vaccine shortly after initial vaccine doses.

### Example 4

This Example demonstrates that SV.Spike+αOX40 vaccinated mice are characterized by a unique T cell transcriptome signature profile after prime vaccine doses.

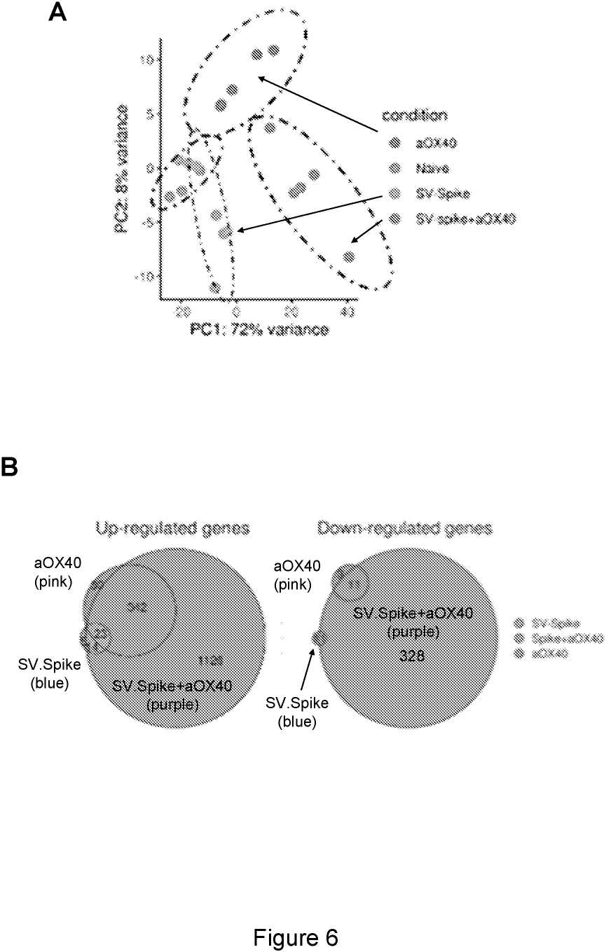

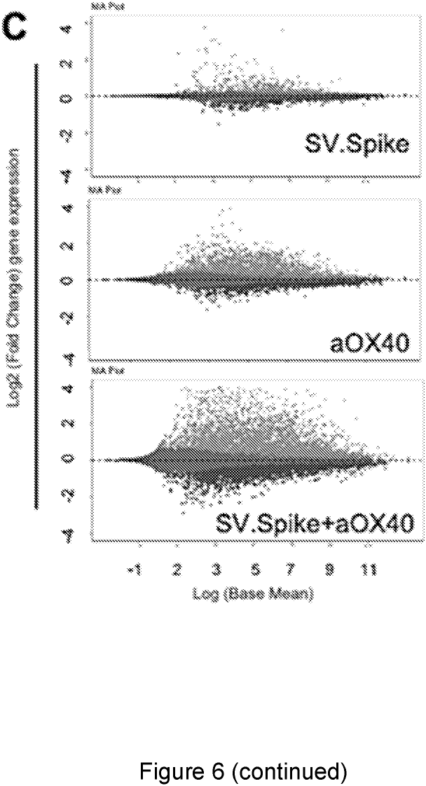

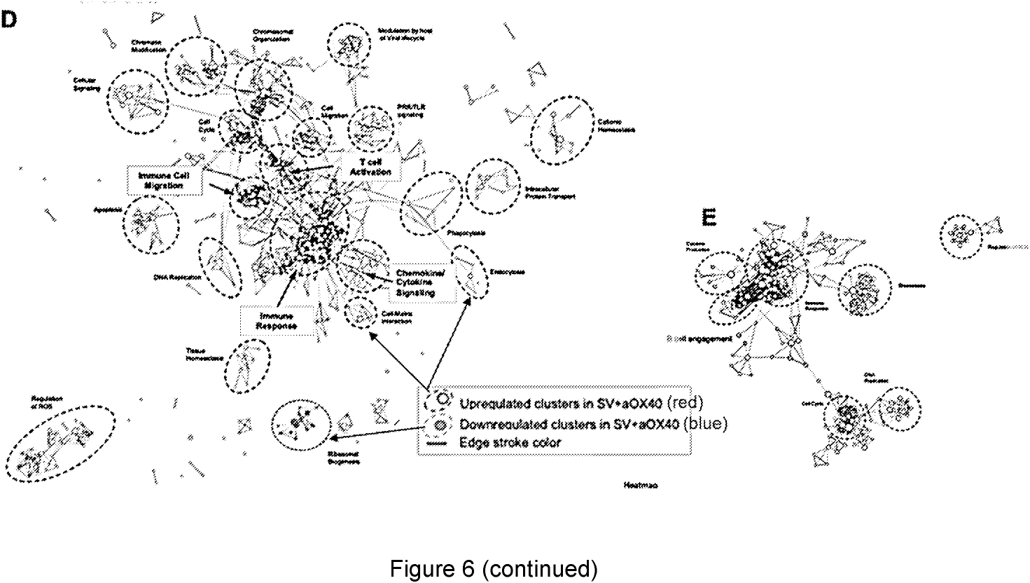

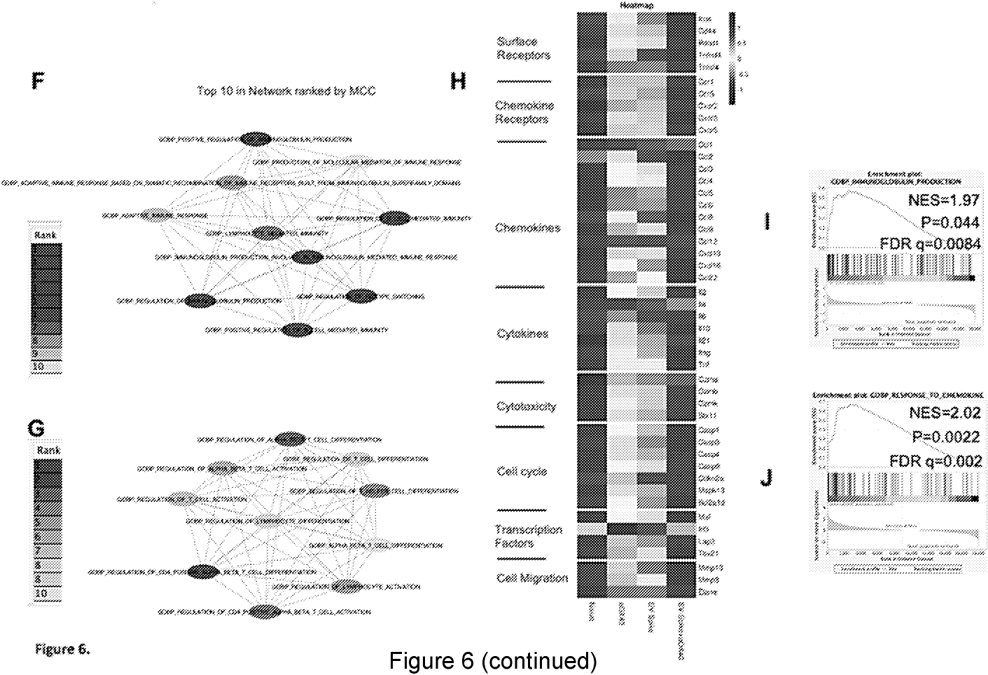

To reveal the molecular profile of SV.Spike vaccine induced T cell responses, we isolated T cells 7 days after prime vaccine doses from spleens of mice from SV.Spike and/or αOX40 vaccinated groups and naive group. We then performed mRNA deep sequencing (RNAseq) and network analysis (FIG. 6). Principal-component analysis (PCA) showed a distinct segregation between combined SV.Spike and αOX40 vaccination and all other groups (FIG. 6A). These data suggest, that SV.Spike and αOX40 induces a distinct T cell response. Indeed, we next looked at gene expression profiles of naive versus SV.Spike and/or αOX40 and we found that naive versus SV.Spike+αOX40 markedly showed the highest amount of uniquely upregulated and downregulated differentially expressed genes (DEGs) with 1,126 upregulated DEGs (left) and 328 uniquely downregulated DEGS (FIG. 6B). Overall, in all groups more genes were significantly upregulated than downregulated (FIGS. 6B-C). These data suggest that SV.Spike+αOX40 changes the transcriptome signature of T cells. We performed Gene Ontology (GO) functional enrichment analysis (also Gene Set Enrichment Analysis, GSEA) of DEGs and network analysis from naïve mice versus SV.Spike+αOX40 (FIG. 6D) and naïve versus SV.Spike only (FIG. 6E) immunized mice to determine key pathways and intersections of these pathways. The majority of pathways were upregulated in T cells isolated from mice immunized with SV.Spike+αOX40 with the exception of one cluster downregulated (ribosomal biogenesis). The upregulated pathways in the combination immunized mice were dominated by immune response, T cell activation, chemokine/cytokine signaling, immune cell migration, DNA replication, chromosomal organization, cell cycle regulation, and chromatin modification that formed the central nodes of this network (FIG. 6D). SV.Spike single agent immunized mice showed a smaller network of seven upregulated pathways including a main cluster of immune response closely connected to a cluster for B cell engagement, a small cluster of cytokine production, chemotaxis, cell cycle, DNA replication, regulation of ROS (FIG. 6E).

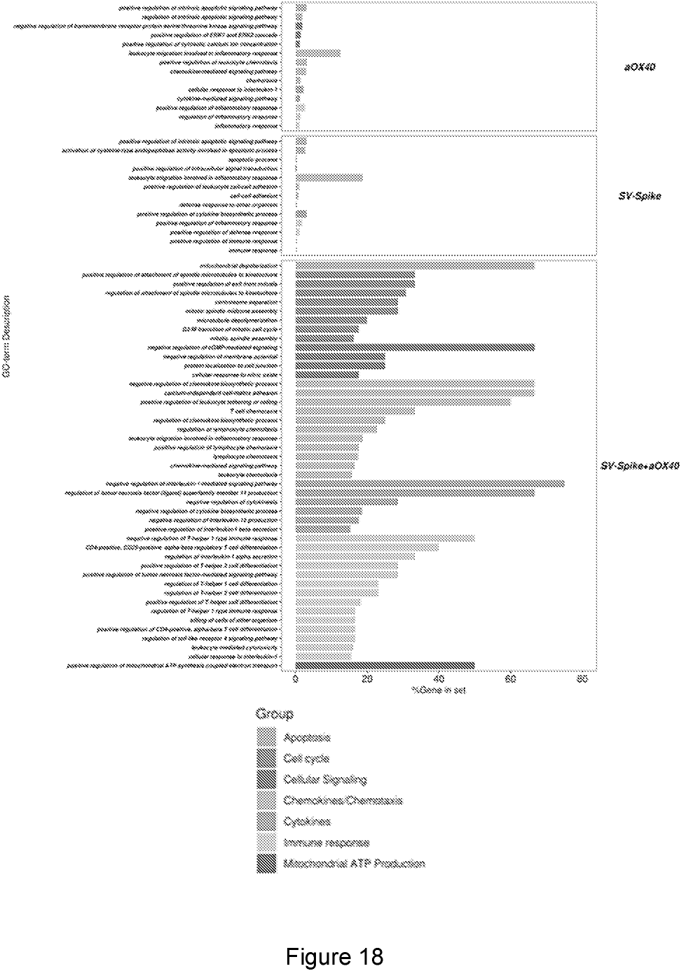

We next identified the top 10 hub GO terms by employing the Maximal Clique Centrality (MCC) for SV.Spike (FIG. 6F) and SV.Spike+αOX40 (FIG. 6G) immunized mice. We found that top 10 hub GO terms in SV.Spike only immunized mice were a selected network cluster of B cell stimulation and Immunoglobulin regulating pathways compared to the combination that represents a cluster of lymphocyte activation and differentiation regulating pathways. Additionally, we performed Protein Association Network Analysis using STRING to identify DEG-encoded protein-protein interactions (PPIs). Significantly upregulated DEGs (>2 fold) in T cells of SV.Spike and/or αOX40 vaccinated mice compared to naive were analyzed to assess overrepresentation of Gene Ontology (GO) categories in Biological Processes in all groups (FIG. 18). GO Biological Processes (Strength ≥1; p<0.05) identified by STRING for each group were assigned to one of 7 clusters (apoptosis, light green; cell cycle, red; cellular signaling, dark blue; chemokines/chemotaxis, yellow; cytokines, pink; immune response, light blue; mitochondrial ATP production, dark green). Each GO Biological Process term is defined by one gene set. The amount of contributing DEGs from mice immunized with SV.Spike and/or αOX40 in each gene set is shown as percentage. We identified fourteen biological processes for αOX40, thirteen for SV.Spike and forty-five for the combination vaccine strategy. We found cell-cycle related processes solely in the SV.Spike+αOX40 combination. The highest amount of chemokines/chemotaxis related processes was observed in the combination (eleven) compared to αOX40 (four) and SV.Spike (four) alone. Six cytokines related pathways were upregulated in the combination versus SV.Spike (one) and αOX40 (two) and fourteen immune response related terms were upregulated in the combination versus SV.Spike (four) and αOX40 (three). Overall, the percentage of DEGs (>2 fold) that contribute to each biological process was highest in the combination vaccinated group compared to SV.Spike and αOX40 alone. Top 20 ranking of selectively enriched GO terms in the GSEA (FDA<0.05) revealed (GO) immunoglobulin production in the SV.Spike group (FIG. 6H) and (GO) response to chemokine in the combination immunized mice group (FIG. 6I, J). We analyzed expression of single signature gene transcripts for each immunized mouse group. We found the highest upregulation of DEGs indicating T cell dependent B cell stimulation for building up humoral immunity against SARS-CoV-2 (ICos, Cxcr5, Il21, Cxcll3), differentiation of Th-1 type effector T cells associated with vaccine effectiveness (Tnfrsf4, Cd44, ICos, Cxcr3, Ccr5, 112, Ifng, Tbx21, Ccl3, Ccl4, Ccl9) and antiviral cytotoxic T cell stimulation for T cell immunity (Gzma, Gzmb, Gzmk) in the SV.Spike immunized mice compared to single agent treated groups (FIG. 6H).

In conclusion, these findings indicate that synergistic SV.Spike+αOX40 vaccine combination successfully changes the transcriptome profile of T cells that is indispensable for building up humoral and T cell immunity.

### Example 5

This Example demonstrates that CD4+ T cell help promotes effector differentiation of cytotoxic T cells.

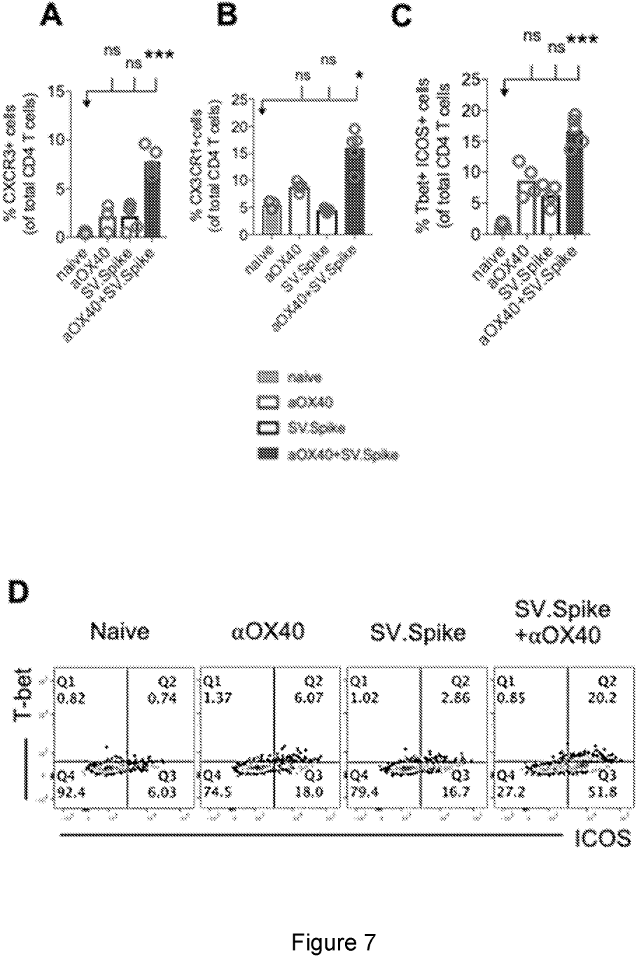

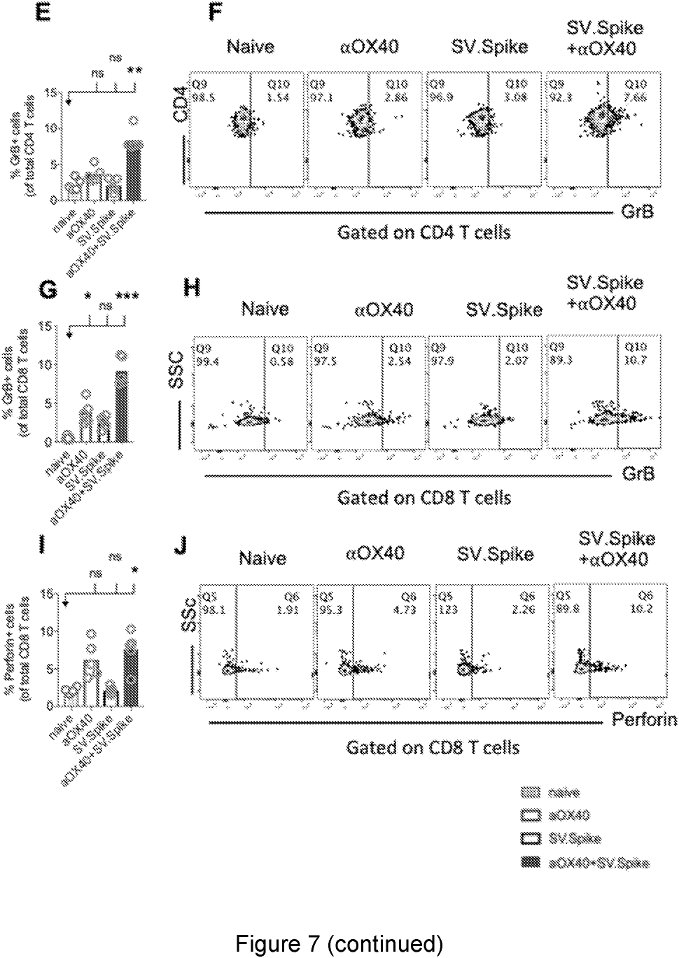

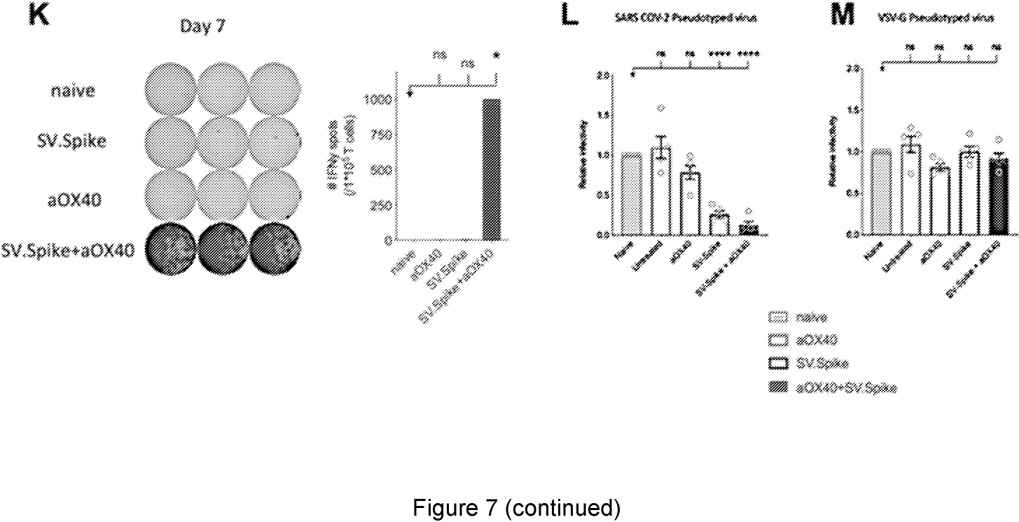

SARS-CoV-2-specific T cells are associated with protective immune responses[54]. Th1- type differentiated effector CD4+ T helper cells promote the development of CD8+ T cells into anti-viral cytotoxic T lymphocytes (CTLs) and functional memory T cells that can be quickly mobilized to directly kill SARS-CoV-2 early on upon re-infection preventing disease in coordination with SARS-CoV-2 specific humoral immune responses. CD4+ T helper cells are critical for success of vaccines and generally work by providing cytokines. We performed flow cytometry analysis to investigate CD4+ T helper differentiation, formation and antiviral cytotoxic effector T cell differentiation in T cells from SV.Spike and/or αOX40 immunized animals (FIG. 7). Chemokine receptors help with the recruitment of type 1 effector and cytotoxic T cells to tissues and lymphoid organs, site-specific activation of memory T cells and T cell clustering around activated antigen presenting cells (APCs). For example, virus-specific cytotoxic T lymphocytes (CTLs) are quickly recruited to influenza-infected lungs by a Th1 response, specifically due to the production of IFNγ[59]. Vaccines mimicking an infection can help to build up tissue specific immunity. Two of these Th 1-type effector T cell chemokine receptors are CXCR3 and CX3CR1. We found a significant increase of CXCR3 and CX3CR1 positive expressing CD4+ T cells (FIGS. 7A, B) from spleens 7 days after administration of prime vaccine doses in the SV.Spike+αOX40 immunized mice group indicating effective recruitment and mobility of generated Th1-type effector T cells. Immunophenotyping by flow cytometry revealed a 2-fold increase of the transcription factor Tbet and immune costimulatory molecule ICOS-double-positive Th1-type effector CD4+ T cells compared with single agent vaccinated mice. Tbet+ ICOS+ are hallmarks of Th1-type T cell polarization (FIGS. 7C, D).

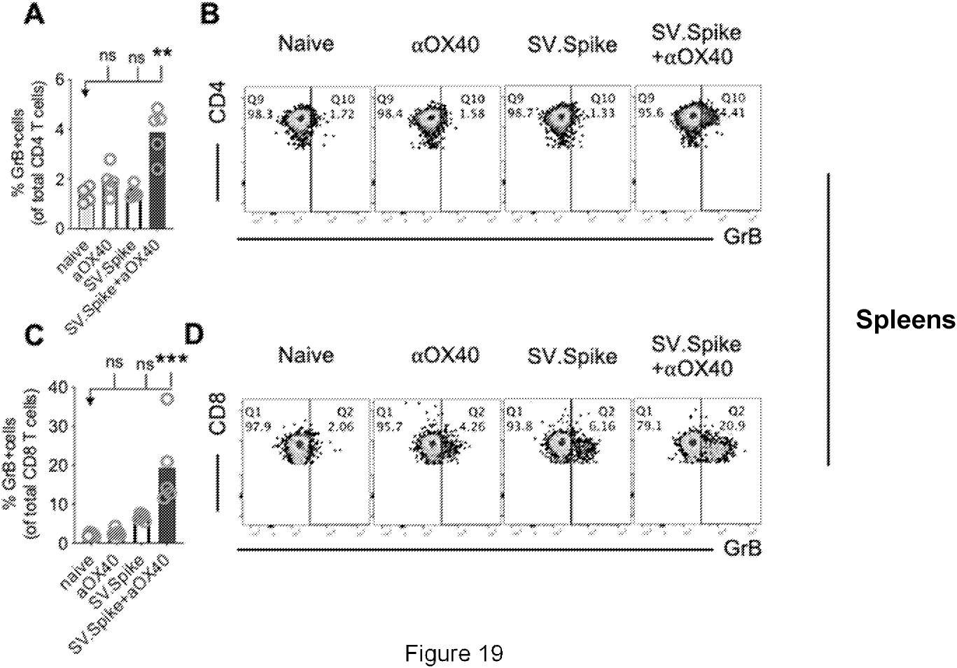

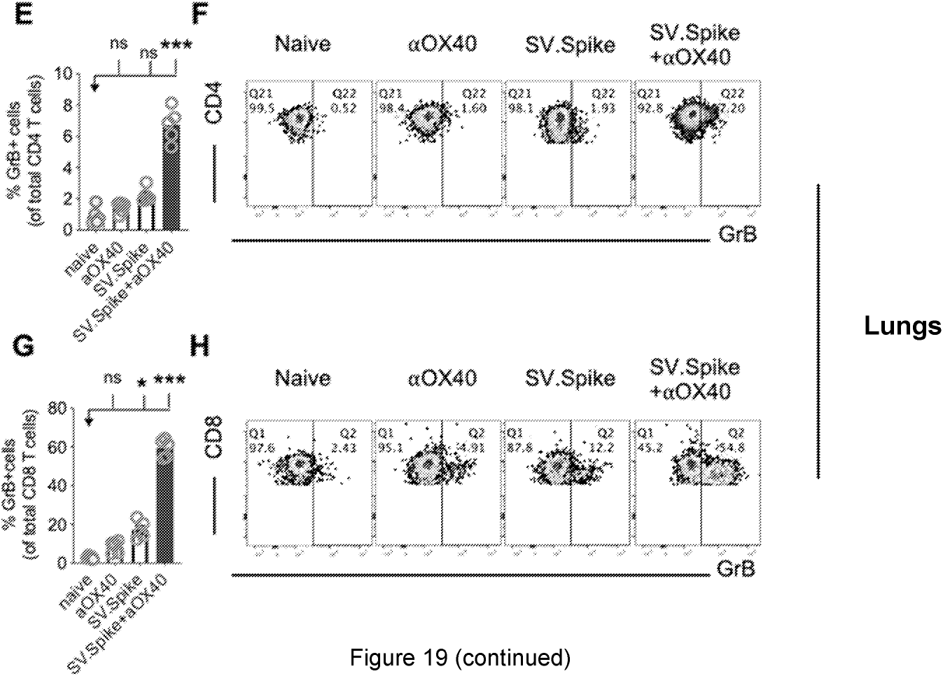

The predominant pathway used by human and murine CD8+ T cells to kill virus-infected cells is granule exocytosis, involving the release of perforin and GrB. It is known from influenza vaccine research that GrB correlates with protection and enhanced CTL response to influenza vaccination in older adults[60]. We looked at CTLs after day 7 of prime doses and found that combination immunization significantly increased differentiation of CTLs indicated by GrB expression (FIGS. 7E-H) and perforin (FIGS. 7I-J) upregulation within one week after initial vaccine doses. Seven days after mice groups received booster doses that were administered on day 14, we found a robust 10-fold upregulation of GrB+ positive CD8+ T cells indicating successful vaccine elicited differentiation of cytotoxic T cells (FIG. 19).

Interestingly, it has been reported that cytotoxic CD4+ T cells can compensate for age related decline of immune cell protection such as B cell loss and a less robust antibody response[61]. Strikingly, we found in SV.Spike+αOX40 immunized mice showed a significant increase of cytotoxic CD4+ T cells indicating that the presently described vaccine not only induced Th1-type CD4+ T helper functions but has the potential to improve direct CD4+ T cell mediated virus-killing, thus, adding an extra layer to immune protection against SARS-CoV-2 in more vulnerable older populations. One important early feature of response to the SV.Spike+αOX40 immunization is a strong interferon-gamma (IFNγ) secretion (FIG. 7K), which is associated with polarization to Th1-type effector cells and cytotoxic T cells. In order to investigate the recruitment and specificity in CTLs to prevent SARS-CoV-2 cell entry, we analyzed the potential of T cells isolated from SV.Spike and/or αOX40 immunized and naive mice on day 7 after prime doses to block the infection of HEK293T cells with SARS-CoV-2-spike expressing, luciferase-encoding pseudovirus. VSVG expressing, luciferase-encoding pseudovirus was used as control. We found, splenic T cells from SV.Spike and SV.Spike+αOX40 mice potently inhibited infection with SARS-CoV-2 pseudotyped lentivirus (FIG. 7L) compared to control (FIG. 7M). In conclusion, SV.Spike+αOX40 activated T cells display a Th-1 effector phenotype that promotes effector differentiation and direct T cell mediated cytotoxicity against SARS-CoV-2 spike within one week after prime vaccine doses.

### Example 6

This Example demonstrates that SV.Spike in combination with αOX40 drives metabolic activation of B cells and T cell dependent B cell support.

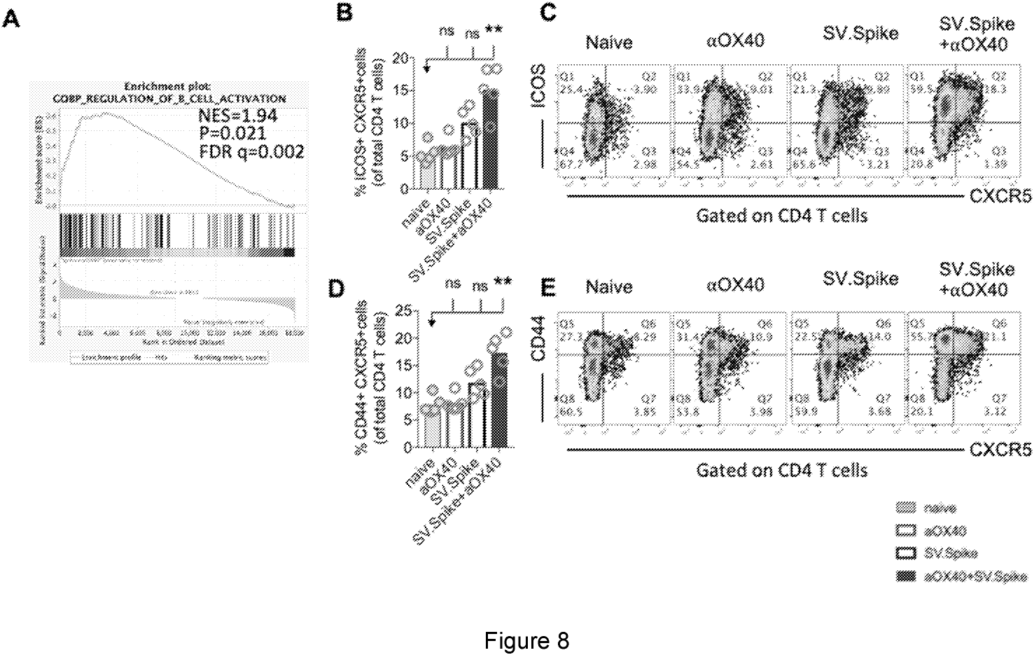

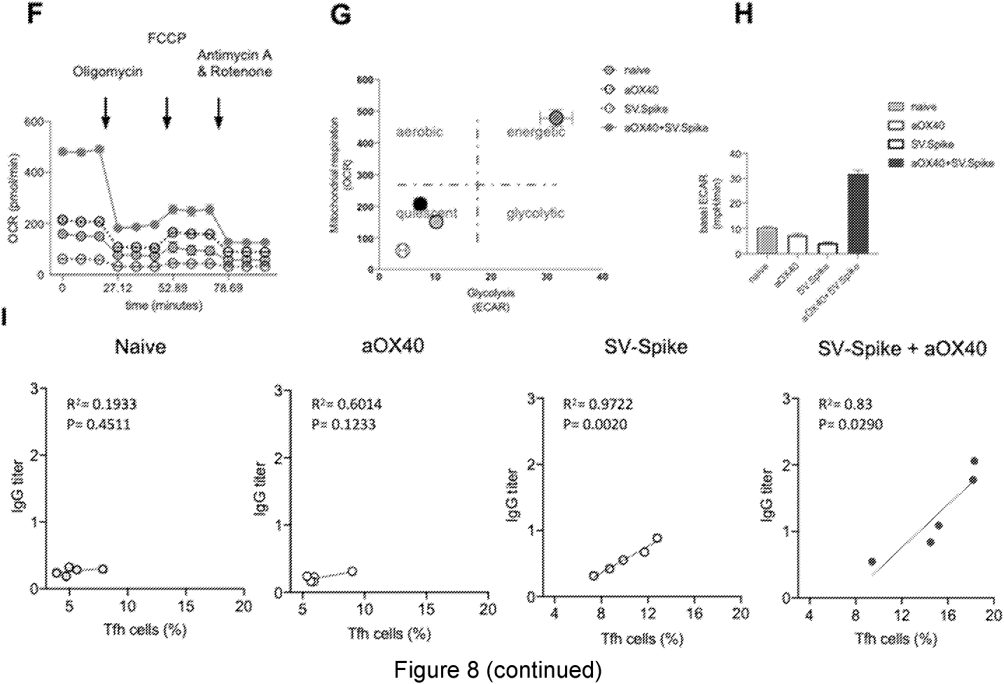

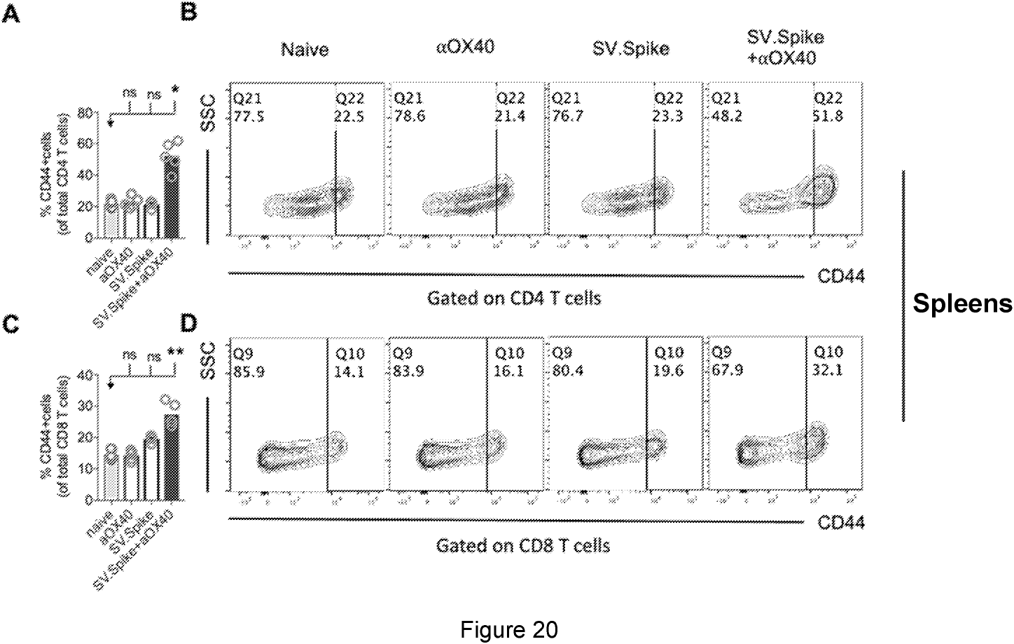

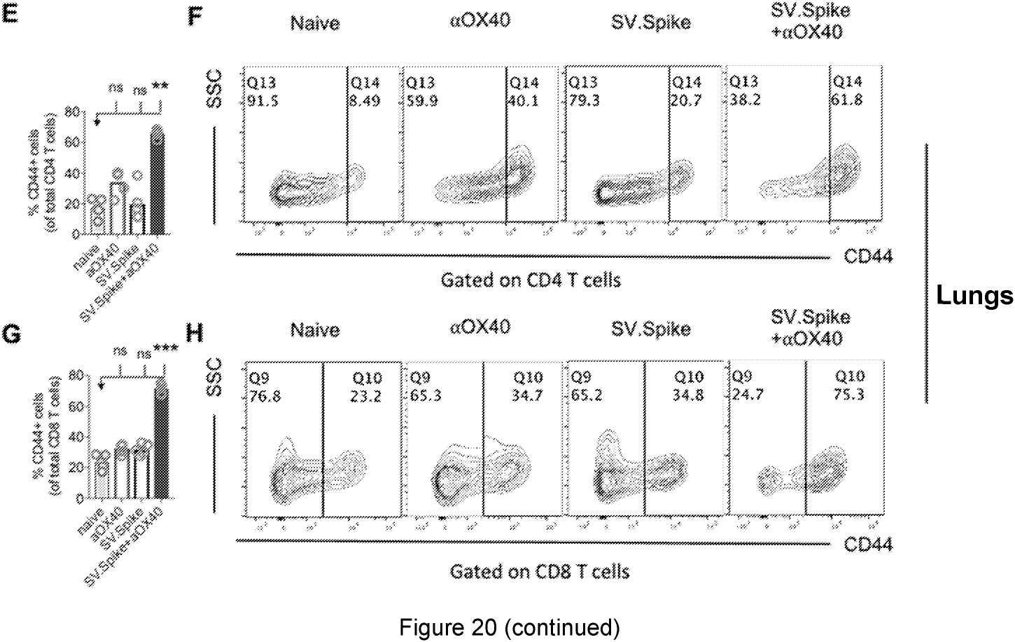

Almost all durable neutralizing antibody responses as well as affinity matured B cell memory depend on CD4+ T cell helper. GSEA of RNAseq data between T cells from the SV.Spike+αOX40 vaccinated and naive group one week after prime vaccine doses revealed selective enrichment of the gene set characteristic for activation of B cells (FIG. 8A) (p<0.05). To test if SV.Spike combination with αOX40 selectively regulates T cell dependent B cell activation, we investigated CD4+ T cell activation and differentiation in mice vaccinated with SV.Spike and/or αOX40 one week after booster vaccine doses by flow cytometry analysis. We found that SV.Spike+αOX40 immunized mice had a 3-fold significant increase of overall CD44+positive splenic CD4+ T cells compared to naive mice (FIG. 20). We next analyzed follicular CD4+ T helper (Tfh) cells that are a subset of CD4+ T cells required for most IgG responses promoting high-quality neutralizing antibodies and we found a 3-fold increase of ICOS+CXCR5+ (FIGS. 8B, C) and a 2 fold increase CD44+CXCR5+ (FIGS. 8D, E) positive CD4+ T cells in splenocytes from the SV.Spike+αOX40 group indicating Tfh cell differentiation. We isolated B cells from spleens and performed a metabolic flux analysis on day 21 after initial vaccine doses and we found that isolated B cells from SV.Spike+αOX40 immunized mice were metabolically reprogrammed indicating potent vaccine elicited B cell activation. Activated B cells in the combination immunized group experienced a 2.5-fold increase in mitochondrial respiration (FIGS. 8F, G) and glycolysis (FIGS. 8G, H) when compared to B cells isolated from mice spleens that were vaccinated with a single agent or compared to naive mice. Association analysis of the frequencies of Tfh cells with SARS-COV-2 spike IgG antibody titers revealed that Tfh cells positively correlated with the SARS-CoV-2 spike IgG serum levels in the SV.Spike (R² = 0.9722, P=0.002) and SV.Spike+αOX40 group (R² = 0.83, P = 0.0290) with the highest amounts of IgG antibodies and Tfh cells in the combination (FIG. 8I). Taken together, these results indicate SV.Spike+αOX40 vaccine induced the most potent T cell dependent B cell response.

### Example 7

This Example demonstrates that a combination of SV.Spike and αOX40 promotes robust T cell specific immune response in lungs.

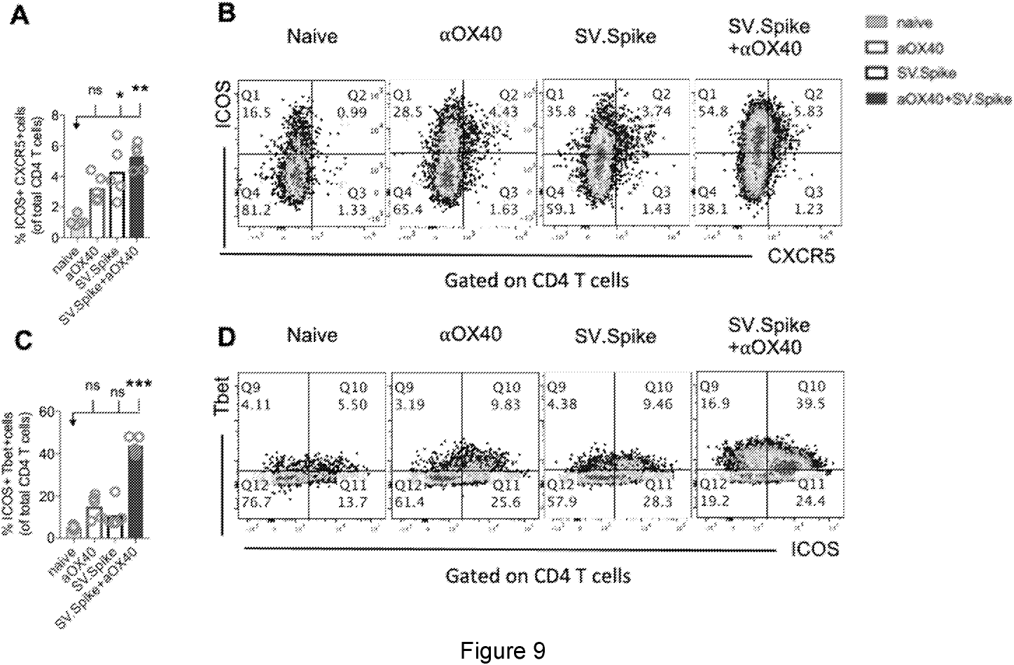

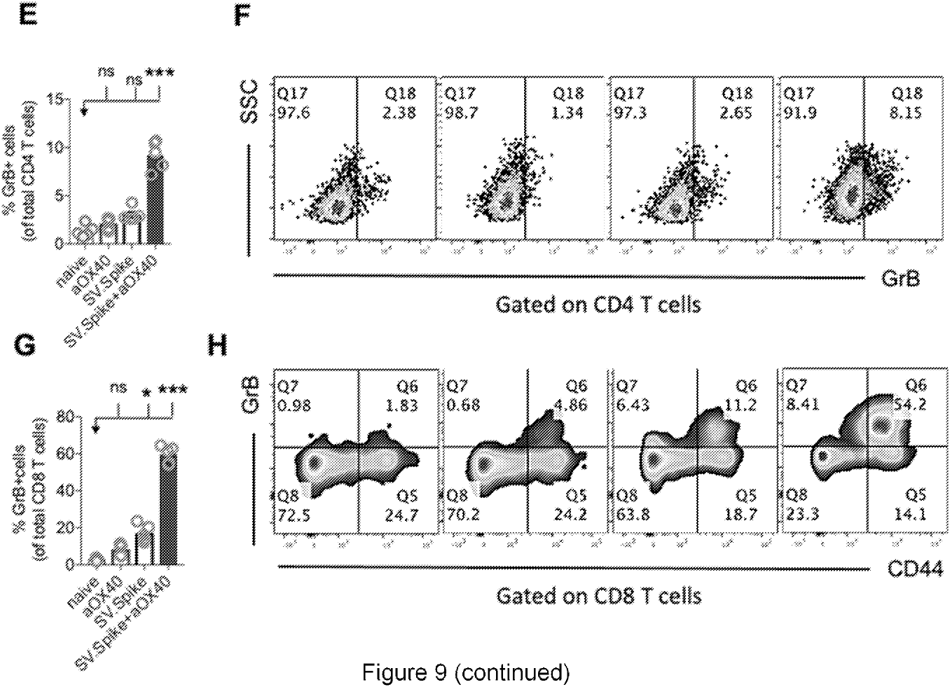

Most vaccines for airborne infectious diseases are designed for delivery via the muscle or skin for enhanced protection in the lung. We investigated if SV.Spike vaccine-induced T cells can readily home most efficiently to the lungs prior to and shortly after pathogen exposure. To address the immune responses in the lungs, we immunized mice with SV.Spike and/or αOX40 and excised PBS-perfused lungs one week after booster doses for single cell suspensions and performed flow cytometry staining (FIG. 9, FIG. 20). We found an increase of ICOS+ CXCR5+ double-positive T helper cells indicating presence of B cell supporting Tfh cells in the SV.Spike single agent and combination immunized group. We further found an increase of Th-1 type effector CD4+ T cells in lungs from combination treated mice indicated by expression of ICOS+Tbet+ double-positive effector CD4+ T cells (FIGS. 9C, D). We next investigated if effector CTLs were successfully recruited into the lungs after 3 weeks of initial vaccine administration. While we found the highest increase of differentiated cytotoxic CD4+ T and CD8+ T cells in lungs from the combination treated group (FIGS. 9E- H, FIG. 19), we observed a significant increase of differentiated cytotoxic CD8+ T cells homing in the lungs of the SV.Spike single agent immunized group, although this increase was less pronounced compared to the combination group. These data indicate a successful recruitment of vaccine mediated antiviral Th1-type effector T cells to the lungs.

### Example 8

This Example demonstrates that SV.Spike and αOX40 promotes CD4+ T cell memory formation and long-term protection upon re-challenge with SARS-CoV-2 Spike antigen.

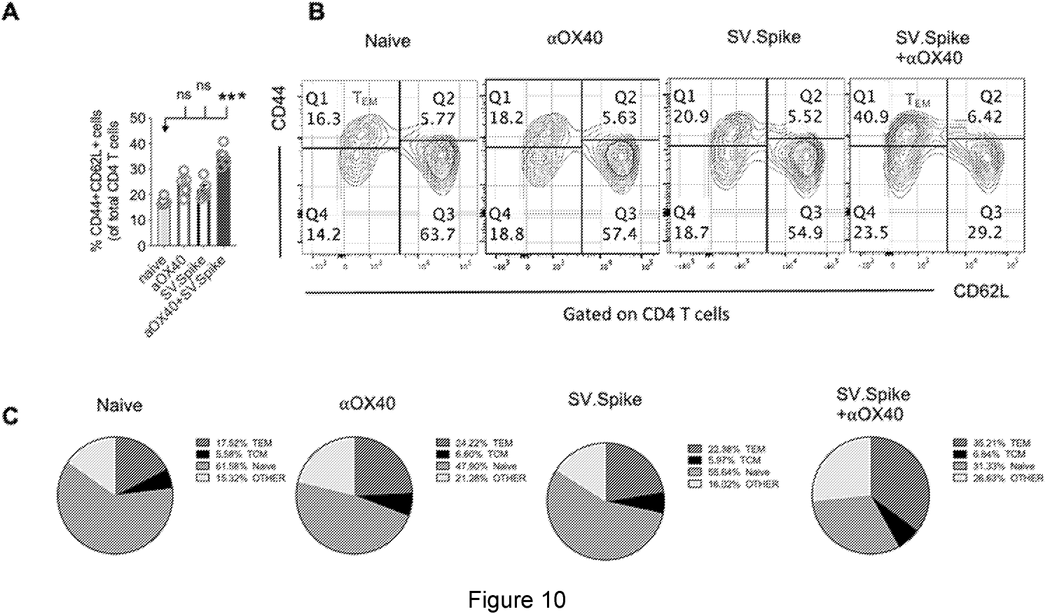

Boosting both, local and systemic memory T cell response is a useful strategy to achieve long term immunity. We analyzed development of T cell memory in spleens fourteen weeks after initial prime vaccine doses of SV.Spike and/or αOX40 prime-boost immunized mice by flow cytometry. We found that mice in the SV.Spike+αOX40 combination group developed significant effector CD4+ T memory indicated by CD44+ CD62L+ double-positive CD4+ T cells (FIGS. 10 A-C) compared with naive mice, reiterating the importance of the combination vaccination in generating strong immune responses memory protection from infection and/or disease against SARS-CoV-2.

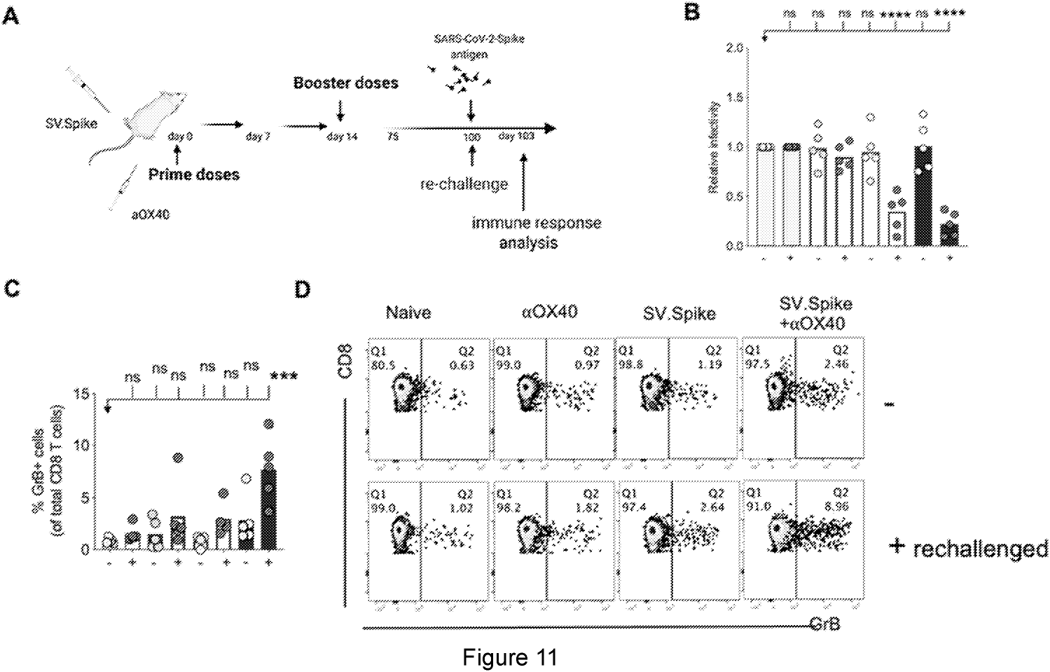

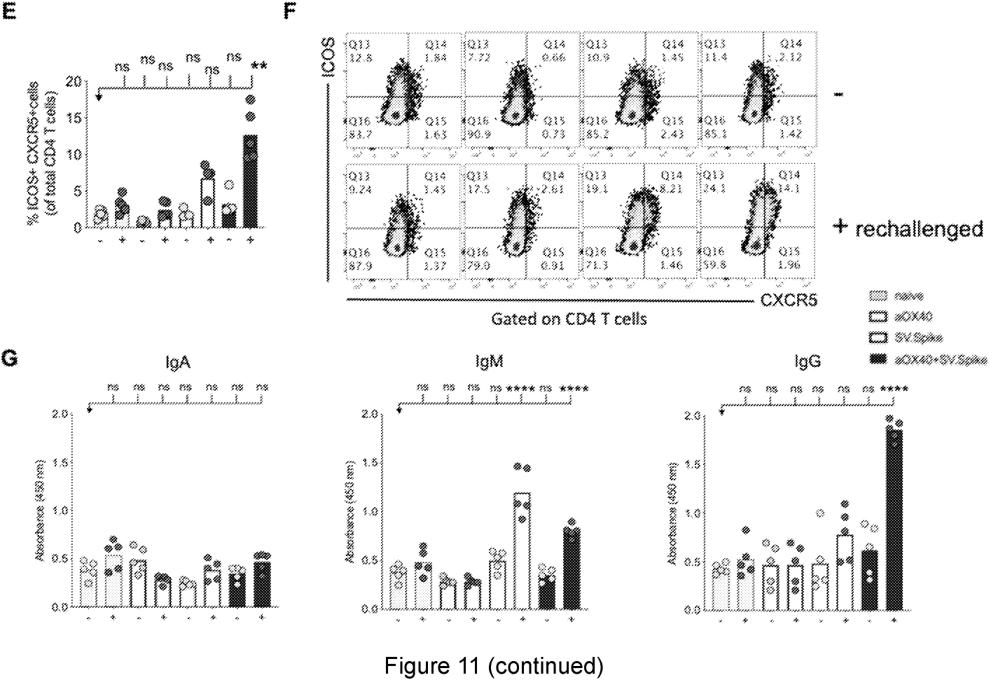

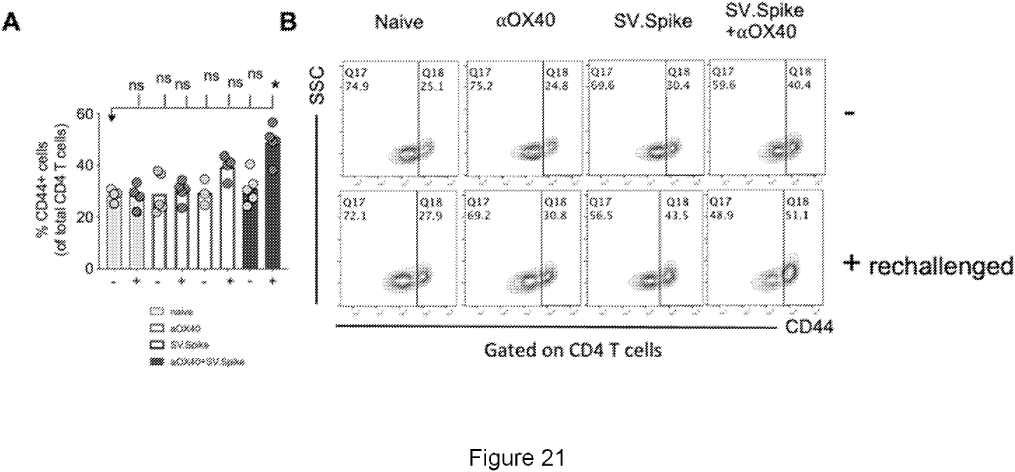

To further explore the long-term protection efficacy of our SV.Spike vaccine against SARS-CoV-2 virus challenge, C57BL/6J mice (n = 5 each group) received prime and boost immunizations of αOX40, SV.Spike and/or αOX40 and placebo (naive group) via the i.p. route. At day 100 post-immunization, we additionally administered one dose of SV.Spike, to recapitulate Spike antigen endogenous entry through SV vector injection (FIG. 11A). Spleens or sera from re-challenged mice were collected 3 days after SARS-CoV-2 spike antigen injection and processed for T cell response analysis (FIGS. 11B-F, FIG. 21) and detection of specific anti-spike protein IgA, Ig and IgG isotypes by ELISA (FIG. 11G). The SARS-CoV-2 pseudotyped lentivirus infectivity assay revealed that mice immunized with SV.Spike or SV.Spike and αOX40 are effective in reactivating circulating cytotoxic T cells (CTLs) upon challenge with Spike antigen (FIG. 11B). CTLs reactivation was also observed by flow cytometry as indicated by granzyme B upregulation in mice receiving combination vaccination (FIGS. 11C, D). Moreover, immunophenotyping analysis showed that CXCR5-ICOS-double-positive Th1-type effector CD4+ T cells were strongly rebooted in re-challenged mice receiving SV.Spike combination vaccination compared to the same group of unchallenged mice (FIGS. 11E, F).

Antibody response analysis showed that immunization with SV.Spike or SV.Spike+αOX40 followed by Spike antigen injection induced strong production of IgM antibodies compared to the mice which did not received the antigen and the Naive groups, and that was particularly evident in mice vaccinated with SV.Spike (FIG. 11G). Strikingly, we noticed that combination of SV.Spike and αOX40 followed by challenge with antigen stimulated a high peak of Spike-specific IgG antibodies levels, which were about 4 times higher than the IgG levels of unchallenged mice and control group. No significant difference in the Spike-specific IgG response was detected in SV.Spike or single αOX40 re-challenged mice compared to the respective unchallenged mice and the control groups, whereas no SARS-CoV-2 spike-specific IgA were not detected in any of the groups (FIG. 11G). Together, these data suggest that combination vaccination with SV.Spike and αOX40 conferred remarkably long-term and specific protection against SARS-CoV-2 infection by eliciting a durable humoral and T-cell response.

### Example 9

### Discussion of Examples

Despite promising results of early clinical trials of several vaccine candidates against SARS-CoV-2, there are still concerns regarding both safety and durability of the immune responses. Consequently, the Examples above demonstrate development of an improved vaccine. This vaccine is considered, without intending to be bound by any particular theory, to be effective after one or two immunizations, conferring long-term protection to target populations such as the elderly or immunocompromised individuals, and reducing onward transmission of the virus to contacts[65].

It is expected, based on the foregoing description, that new constructs can be made rapidly with synthetic design of the insert, and readily adapted to SARS-CoV-2 variants. Moreover, when new virus species emerge, the described vaccine platform can be rapidly adapted to combat emerging viruses.

Sindbis virus and other alphaviruses have a natural tropism for lymphatic tissues and dendritic cells, relative resistance to interferon, high expression levels, lack of pre-existing anti-vector immunity in most human and animal populations, and efficient production of methodology in cell lines, with an accepted regulatory pedigree[72].

Neutralizing antibodies (NAbs) have conventionally been the desired outcome of vaccination, as they are capable of intercepting and neutralizing microbes and their components, as well as eliciting destructive anti-microbial innate immune responses[73]. Nonetheless, humoral immunity can decline over time and, as seen with influenza, can only last as short as one season. Many newer vaccines and vaccines in development are also designed to generate T cell responses that have the potential to help the antibody response, promote long-term immune memory, have direct effector functions themselves, or activate innate effector cells such as macrophages and neutrophils[45; 74].

The present disclosure provides a Sindbis-based Spike-encoding RNA vaccine against SARS-CoV-2 and demonstrates that immunization with SV vector expressing SARS-CoV-2 Spike along with a costimulatory agonistic αOX40 antibody induced a synergistic T cell and antibody response and provided complete protection against authentic SARS-CoV-2 challenge in hACE2 transgenic mice. It is expected that this approach will boost tissue specific immunity and immune memory against the described viruses and could protect for several seasons or years. As a viral vector, we found that a Sindbis vector expressing SARS-CoV-2 Spike antigen in combination with αOX40 markedly improves the initial T cell priming, compared with the viral vector alone, which results in a robust CD4+ and CD8+ T cell response and stable SARS-CoV-2 specific neutralizing antibodies. The vaccine efficiently elicits effector T cell memory in respiratory tissues with a potential for long lasting protection against COVID19, which might extend for several years, through multiple beneficial mechanisms. It protects against infection with authentic, SARS-CoV-2 preventing morbidity and mortality.

αOX40 controls survival of primed CD8+ T cells and confers CTL-mediated protection[31; 75]. CTLs are a critical component of the adaptive immune response but during aging, uncoordinated adaptive responses have been identified as potential risk factors that are linked to disease severity for the outcome of COVID19 patients. It is known from influenza vaccine research that Granzyme B correlates with protection and enhanced CTL response to influenza vaccination in older adults. We looked at cytotoxic T cells (CTLs) and found that combination vaccination significantly increased CD8+ cytotoxic T cells indicated by granzyme B and perforin upregulation. Almost all durable neutralizing antibody responses as well as affinity matured B cell memory depend on CD4+ T helper cells. We found in combination vaccinated mice a significant increase of cytotoxic CD4+ T cells indicating that the presently described vaccine not only induced CD4+ T helper functions but has the potential to improve direct CD4+ T mediated virus-killing adding an extra layer to long-term immunity/protection in more vulnerable older populations.

Virus-specific CTLs are quickly recruited to influenza-infected lungs by a Th1 response, specifically due to the production of IFNγ[59]. IFNγ regulates various immune responses that are critical for vaccine-induced protection and has been well studied[76; 77]. In a clinical trial of the now approved BNT162b1, IFNγ secreting T cells increased in participants 7 days after boost [45]. In this regard, one important early feature of the response to the SV.Spike+αOX40 immunization is a strong interferon-gamma (IFNγ) secretion. We found a significant increase of CXCR3 and CX3CR1 positive expressing CD4+ T cells, indicating effective recruitment and mobility of generated effector Th1 type T cells in mice. This recruitment positively correlates with vaccine induced long-term immune protection and generation of neutralizing antibodies against SARS-CoV-2.

Both humoral and cell-mediated immune responses have been associated with vaccine-induced protection against challenge or subsequent re-challenge after live SARS-CoV-2 infection in recent rhesus macaque studies [78; 79] and there is mounting evidence that T-cell responses play an important role in COVID-19 mitigation[3; 80; 81]. We demonstrated that two doses of SV.Spike with or without αOX40 candidate vaccines induced neutralizing antibody titers in all immunized mice, with a strong IgG response in the mice receiving combination vaccination. Moreover, the Examples show that SV.Spike+αOX40 skewed Tfh cells toward CXCR5⁺ Tfh differentiation, which positively correlated with the magnitude of IgG isotype response. These findings indicate that the induction of CXCR5⁺ Tfh cell differentiation through vaccination may be beneficial for eliciting broad and specific NAb responses. Importantly, the synergistic activity of combination vaccination elicited antibodies that were able to efficiently neutralize SARS-CoV-2 pseudotyped lentivirus in all the mice tested. In addition, we show SV-Spike-based re-challenge in mice immunized with combination vaccination led to enhanced cytotoxic reactivation of T cells and increased IgG seroconversion and response, and provided protection against re-challenge, reiterating the importance of the involvement of both humoral and cellular immune responses in SARS-CoV-2-mediated immunity.

The described SV.Spike platform has the advantage that it is inexpensive, stable, and easy to produce when given the benefit of the present disclosure . Cost projections based on using the described processes for production of a SV based vaccine are in line with or below costs per dose for other vaccines in use today. Moreover, unlike other mRNA vaccine candidates this viral platform does not require a cold-chain during transportation and storage. It can be easily reconstituted after lyophilization process and is suitable for rapid adaptation such that potential new viruses/threats in an emerging outbreak can be rapidly targeted[82]. Thus, for emerging pathogens like SARS-CoV-2, the described SV platform can be an efficient and cost-effective alternative to the traditional large-scale antigen production or technology platforms that require extended time for implementation.

As shown in this disclosure, SV.Spike can be applied alone or can be combined with immunomodulatory reagents like αOX40 in a remarkably efficient prime-boost regimen. The disclosure includes a combined SV.Spike + αOX40 coding single vector.

### Example 10

### Material and Methods

### Cell Lines

Baby hamster kidney (BHK) and HEK293T cell lines were obtained from the American Type Culture Collection (ATCC). 293T-ACE2 cell line was obtained from BEI Resources.

BHK cells were maintained in minimum essential α-modified media (α-MEM) (Corning CellGro) with 5% fetal bovine serum (FCS, Gibco) and 100 mg/ml penicillin-streptomycin (Corning CellGro). 293T and 293T-ACE2 cells were maintained in Dulbecco’s modified Eagles medium containing 4.5 g/l Glucose (DMEM, Corning CellGro) supplemented with 10% FCS, 100 mg/ml penicillin-streptomycin. All cell lines were cultured at 37° C. and 5% CO2.

### SV Production

SV.Spike expressing vector was produced as previously described[38; 39; 83; 84]. Briefly, plasmids carrying the replicon (pT7-SV-Spike) orT7-DMHelper RNAs were linearized with XhoI. In vitro transcription was performed using the mMessage mMachine RNA transcription kit (Invitrogen Life Sciences). Helper and replicon RNAs were then electroporated into BHK cells and incubated at 37° C. in αMEM supplemented with 10% FCS. After 12 hours, the media was replaced with OPTI-MEM (GIBCO-BRL) supplemented with CaCl2 (100 mg/l) and cells were incubated at 37° C. After 24 hours, the supernatant was collected, centrifuged to remove cellular debris, and frozen at -80° C. Vectors were titrated as previously described [85].

### Pseudotyped Lentivirus Production

SARS CoV-2 pseudotyped lentiviruses were produced by transfecting the HEK293T cells with the pLenti-Puro vectors (Addgene) expressing Luciferase or β-Galactosidase, with pcDNa3.1 vector expressing SARS-CoV-2 Spike (BEI repository) and the helper plasmid pSPAX2 (Addgene). The VSV-G and empty lentiviruses were produced by replacing pcDNA3.1-Spike with pcDNA3.1-VSV-G or pcDNA3.1 empty vector, respectively (Addgene). The transfections were carried out using the Polyethylenimine (PEI) method with the ratio at PEI:pLenti:pcNDA3.1-Spike:pSPAX2 = 14:2:2:1 or PEI:pLenti:pVSV-G/pcNDA3. 1:pSPAX2 = 10:1:0.5:3. The virus-containing medium was harvested 72 hours after transfection and subsequently pre-cleaned by centrifugation (3,000 g) and a 0.45 µm filtration (Millipore). The virus-containing medium was concentrated by using a LentiX solution (TakaraBio) a 10:1 v/v ratio and centrifuged at the indicated RCF at 4° C. After centrifugation, the supernatant was carefully removed and the tube was drained on the tissue paper for 3 minutes. Dulbecco’s modified Eagles medium containing 4.5 g/l Glucose (DMEM) was added to the semi-dried tube for re-suspension and then stored at -80° C.

### Detection of SARS-CoV-2 Spike Pseudotyped Lentivirus Infectivity

Luciferase- and nLacZ-encoding SARS CoV-2 Spike or VSV-G pseudotyped lentivirus titers were determined making serial dilutions of the vectors in DMEM and infect 293T/ACE2 cells pre-plated in 96-well culture plates (10⁴ cells/well) and 24 h later, fresh media was added. For Luciferase-encoding pseudotype, cells were lysed 72 h later using cell lysis buffer and lysates were transferred into fresh 96-well luminometer plates, where luciferase substrate was added (Thermo Fisher), and relative luciferase activity was determined (FIG. 15C). For nLacZ-encoding pseudotypes, cells were washed with PBS and stained for 16 h at 37° C. with X-Gal Solution [1 mg/ml X-Gal in PBS (pH 7.0) containing 20 mM potassium ferricyanide, 20 mM potassium ferrocyanide and 1 mM MgC12] (FIG. 15D). Vector titers refer to the number of infectious particles (transducing units per milliliter of supernatant [TU/mL] and were estimated as the last dilution having detectable reporter activity. Correct assembling of pseudotypes was assessed by western blot following standard protocol, to detect the expression of SARS-CoV-2-Spike and p24 proteins. SARS-CoV-2 Spike (BPS Bioscience) and p24 (Abcam) recombinant proteins were used as positive controls (FIGS. 15A, B).

### In Vivo Experiments

All experiments were performed in accordance with protocols approved by the Institutional Animal Care and Use Committee of New York University Grossman School of Medicine. Six to 12-week old female C57BL/6J albino mice (B6(Cg)-Tyr<c-2J>/J,Cat#000058) and mice expressing the human ACE2 receptor2 : B6(Cg)-Tg(K18-ACE2)2Prlmn/J Hemizygous or non-carrier controls (Cat#034860) were purchased from Jackson Laboratory.

### ABSL3 Experiments Using SARS-CoV-2 Coronavirus

Three weeks after prime and boost vaccination doses, B6(Cg)-Tg(K18-ACE2)2Prlmn/J and non-carrier control mice were challenged with 10⁴ particles of SARS-CoV-2 Coronavirus via the intranasal (i.n.) route (FIG. 4F). We recorded daily the body weight of each mouse after infection for a total of 14 days. The New York University Grossman School of Medicine (NYUSOM) Animal Biosafety Level 3 (ABSL3) Facility, located on the third floor of the Alexandria Center for Life Science West Tower, is a 3,000 sq.ft. high-containment research facility under the responsibility of the Office of Science & Research and its Director of High-Containment Laboratories. It has been designed and it is operated in compliance with the guidelines of the Centers for Disease Control and Prevention (CDC) and the National Institutes of Health (NIH). All research and non-research operations are governed by institutional standard operating procedures (SOPs). As per those SOPs, all users undergo specific training and require medical and respiratory protection clearance. The facility and its SOPs are re-certified by an outside consultant on a yearly basis. The NYUSOM ABSL3 has also been registered with the Department of Health and Mental Hygiene of the city of New York since March 2017.

### Mouse Vaccination and Serum Collection

Mice were i.p. immunized with SV.Spike (10⁷ TU/ml) in a total volume of 500 µl was injected i.p. into the left side of the animal. The immunostimulatory αOX40 antibody (clone OX-86, BioXCell) was injected i.p. into the left side of the animal at a dose of 250 µg per injection. Mice were boosted once at 2 weeks. Sera were collected at 7 days post-2^{nd} vaccination and used to detect neutralizing activity.

Therapeutic efficacy of vaccines was monitored in two ways: vaccinated B6(Cg)-Tg(K18-ACE2)2Prlmn/J mice that were challenged with SARS-CoV-2 Coronavirus in BSL3 were tested for survival compared to their non immunized control group. Survival was monitored and recorded daily.

### In Vivo Delivery of nLacZ-SARS-CoV-2 Pseudotype and X-Gal Histochemistry

Isoflurane-anesthetized 4-week-old young adult B6(Cg)-Tg(K18-ACE2)2Prlmn/J (hACE2-Tg) mice were dosed intranasally with a 70-µl volume of nLacZ-encoding lentiviral vector (titer 5.18×10³ TU/ml). Isoflurane anesthesia (2.5% isoflurane/1.51 oxygen per minute) and dosing of animals was carried out in a vented BSL-2 biological safety cabinet. For processing of mouse lungs for X-Gal staining of intact tissue, lungs were inflated through the trachea with OCT embedding as described previously[86]. Intact airways were submerged in 0.5% glutaraldehyde for 2 hat 4° C., washed in PBS/1 mM MgCl2 and stained for 16 h at 37° C. with X-Gal Solution [1 mg/ml X-Gal in PBS (pH 7.0) containing 20 mM potassium ferricyanide, 20 mM potassium ferrocyanide and 1 mM MgCl₂].

### Neutralization Experiments

### SARS-CoV-2 Spike-hACE2 Blocking Assay

To measure protective NAbs, COVID-19 convalescent plasma was diluted (1:10) and incubated with recombinant SARS-CoV-2 full-length Spike (BPS Bioscience) for 1 h at 37° C. prior to adding to an ACE2 pre-coated ELISA plates. The NAb levels were calculated based on their inhibition extents of Spike and hACE2 interactions according to the following equation: [(1-OD value of samples/OD value of negative control) × 100%]. A neutralizing antibody against SARS-CoV-2 Spike (Bio Legend) was used as a positive control.

### SARS-CoV-2 Spike Pseudotyped Lentivirus Inhibition Assay

Pseudotyped lentivirus inhibition assay was established to detect neutralizing activity of vaccinated mouse sera and inhibitory ability of antiviral agents against infection of SARS-CoV-2 Spike pseudotyped lentivirus in target cells. Briefly, pseudotyped virus containing supernatants were respectively incubated with serially diluted mouse sera at 37° C. for 1h before adding to target cells pre-plated in 96-well culture plates (10⁴ cells/well). 24 h later, fresh media was added and cells were lysed 72 h later using cell lysis buffer. Lysates were transferred into fresh 96-well luminometer plates. Luciferase substrate was added (Promega), and relative luciferase activity was determined. The inhibition of SARS-COV-2 Spike pseudotype lentivirus was presented as % inhibition.

### Cell-Cell Fusion Assay

The establishment and detection of several cell-cell fusion assays are as previously described [47]. In brief, 293T/ACE2 cells were used as target cells. For preparing effector cells expressing SARS-CoV-2 Spike, 293T cells were transiently co-transfected with pcDNA3.1-Spike and pMAX-GFP or with pMAX-GFP only as control, and applied onto 293T/ACE2 cells after 48 h. Effector and target cells were cocultured in DMEM plus 10% FBS for 6 h. After incubation, five fields were randomly selected in each well to count the number of fused and unfused cells under an inverted fluorescence microscope (Nikon Eclipse Ti-S).

### Inhibition of SARS-CoV-2-Spike-Mediated Cell-Cell Fusion

The inhibitory activity of neutralizing antibodies from immunized mice sera on a SARS-CoV-2-Spike-mediated cell-cell fusion was assessed as previously described[49; 87].

Briefly, a total of 2 × 10⁴ target cells/well (293T/ACE2) were incubated for 5 h. Afterwards, medium was removed and 10⁴ effector cells/well (293T/Spike/GFP) were added in the presence of serum from C57BL/6J immunized mice at 1:100 dilution in medium at 37° C. for 2 h. The fusion rate was calculated by observing the fused and unfused cells using fluorescence microscopy.

### Immunocytochemi Stry

Cell immunocytochemistry was performed as described previously[88]. Briefly, cells were fixed with 4% paraformaldehyde (PFA) for 20 min at room temperature and then the membrane was permeabilized with 0.1% (vol/vol) Triton X-100 (Fisher Scientific). Incubation with blocking solution (5% normal goat serum) was performed at room temperature for 45 min. Anti-mouse SARS-CoV-2-Spike (GTX, 1:100) and anti-rabbit hACE2 (Thermo Fisher, 1: 100) were applied overnight at 4° C. followed by incubation of appropriate secondary antibodies conjugated with fluorophores. Confocal images were captured using the Zeiss LSM-800 system.

### Flow Cytometry

For flow cytometry analysis, spleens were harvested from mice and processed as previously described[39]. Extracted lungs were chopped in small pieces and incubated with a digestive mix containing RPMI with collagenase IV (50 µg/ml) and DNAseI (20 U/ml) for 30 min at 37° C. Spleens and lungs were mashed through a 70-µm strainer before red blood cells were lysed using ammonium-chloride-potassium (ACK) lysis (Gibco). Cells were washed with PBS containing 1% FCS and surface receptors were stained using various antibodies. Fluorochrome-conjugated antibodies against mouse CD3, CD4, CD44, CD38, ICOS, OX40, CD62L, Perforin, Granzyme B and Tbet, CXCR5 were purchased from Biolegend. Fluorochrome-conjugated antibodies against mouse CD8a were purchased from BD Biosciences. Fluorochrome-conjugated antibodies against CXCR3 and Ki67 were purchased from Thermofisher. Stained cells were fixed with PBS containing 4% Formaldehyde. For intracellular staining, the forkhead box P3 (FOXP3) staining buffer set was used (eBioscience). Flow cytometry analysis was performed on a LSR II machine (BD Bioscience) and data were analyzed using FlowJo (Tree Star).

### T and B Cell Isolation

Total T cells were freshly isolated with the EasySep™ mouse T Cell Isolation Kit. Total B cells were freshly isolated with the EasySep™ mouse B Cell Isolation Kit. Isolation of T and B cells were performed according to the manufacturer’s protocols (Stemcell Technologies).

### Enzyme-Linked Immunospot (ELISPOT)

Enzyme-linked immunospot was performed as previously described[39]. Mouse IFNγ ELISPOT was performed according to the manufacturer’s protocol (BD Bioscience). Freshly isolated (1 × 10⁵) T cells were directly plated per well overnight in RPMI supplemented with 10% FCS. No in vitro activation step was included. As positive control, cells were stimulated with 5 ng/ml PMA+1 µg/ml Ionomycin.

### Ex Vivo Cytotoxic Assay

T cells (8 × 10⁵ /mL) from C57BL/6J immunized splenocytes were co-cultured with 293T/ACE2 cells (2 × 10⁴ /mL), previously infected with 3×10⁵ TU of SARS-CoV-2 Luc-SARS-CoV-2 Spike pseudotyped lentivirus. Cells were co-cultured in a 24-well plate for 2 days in 1 mL of RPMI 1640 supplemented with 10% FCS, washed with PBS and lysed with 100 µL of M-PER mammalian protein extraction reagent (ThermoFisher) per well. Cytotoxicity was assessed based on the viability of 293T/ACE2 cells, which was determined by measuring the luciferase activity in each well. Luciferase activity was measured by adding 100 µL of Steady-Glo reagent (Promega) to each cell lysate and measuring the luminescence using a GloMax portable luminometer (Promega).

### Transcriptome Analysis of T Cells

Total RNA was extracted from freshly isolated T cells on day 7 of treatment from spleens using RNeasy Kit (Qiagen). For each group, 5 C57BL/6J mice were used for biological repeats. RNA-seq was done by NYUMC Genome Center. RNA quality and quantity were analyzed. RNAseq libraries were prepared and loaded on the automated Illumina NovaSeq 6000 Sequencing System (Illumina). 1x S1 100 Cycle Flow Cell v1.5, 30 automated stranded RNA-seq library prep polyA selection, per sample.

### RNA-Seq Data Analysis

RNA-seq data were analyzed by sns rna-star pipeline (github.com/igordot/sns/blob/master/routes/rna-star.md). Sequencing reads were mapped to the reference genome (mm 10) using the STAR aligner (v2.6.1d)[89]. Alignments were guided by a Gene Transfer Format (GTF) file. The mean read insert sizes and their standard deviations were calculated using Picard tools (v.2.18.20) (broadinstitute.github.io/picard). The read count tables were generated using subread (v1.6.3)[90], (normalized based on their library size factors using DEseq2[91], and differential expression analysis was performed. To compare the level of similarity among the samples and their replicates, we used principal-component analysis. All the downstream statistical analyses and generating plots were performed in R environment (v4.0.3) (www.r-project.org/). The results of gene set enrichment analysis were generated by GSEA software[92; 93]. The network of Gene Ontology terms was generated by Enrichment Map in Cytoscape. Additional protein-protein functional associations used in this disclosure for bar graphs were retrieved from STRING (/www.string-db.org/, version 11)[94], a well-known public database on several collected associations between proteins from various organisms.

### Measurement of Oxygen Consumption and Extracellular Acidification Rates of T and B Cells

T and B cell metabolic output was measured by Seahorse technology as previously described[95]. Purified T cells from C57BL/6J immunized or control mice were plated at 6x10⁵ cells/well in a Seahorse XF24 cell culture microplate. Oxygen consumption rate (OCR) and extracellular acidification rate (ECAR) were measured using an Agilent Seahorse XFe24 metabolic analyzer following the procedure recommended by the manufacturer (Agilent). For the mitochondrial stress test, 1) oligomycin (1 µM), 2) FCCP (1.5 µM) and 3) rotenone (100 nM) and antimycin A (1 pM) were injected sequentially through ports A, B and C.

### Immunoblot Analysis

Western blot was performed to detect SARS-CoV-2 spike protein in HEK293T cells infected with SV.Spike and in the generated pseudotyped lentivirus. Cells were lysed in M-PER® Mammalian Protein Extraction Reagent (Thermo Fisher) according to the manufacturer’s protocol. Lysates were separated by SDS-PAGE on 4-15% Bio-Rad gels, transferred to polyvinylidene difluoride (PVDF) membranes, blocked in 5% milk in TBS buffer with 0.1% Tween-20 (TBST). Primary antibodies to SARS-CoV-2 Spike (GTX, 1:1000) and p24 (Abcam, 1:1000) were added overnight at 4° C. HRP-conjugated secondary antibodies were added in 5% milk in TBST for 1 h at room temperature. BioRad Imaging System was used for visualization.

### Statistical Analysis

Statistical analysis was performed using GraphPad Prism 7.0 as described in figure legends. All data are shown as mean ± SEM. Figures were prepared using GraphPad Prism 7, Adobe Photoshop and ImageJ Software. Treated groups were compared using a one-way analysis using Prism7 (GraphPad Software) to naive mice. Differences with a P value of <0.05 were considered significant: *P<0.05; **P<0.005; ***P<0.001.

References - This reference listing is not an indication that any of the references are material to patentability.

A.C. Moore, and C.L. Hutchings, Combination vaccines: synergistic simultaneous induction of antibody and T-cell immunity. Expert Rev Vaccines 6 (2007) 111-21.

Y. Peng, A.J. Mentzer, G. Liu, X. Yao, Z. Yin, D. Dong, W. Dejnirattisai, T. Rostron, P. Supasa, C. Liu, C. Lopez-Camacho, J. Slon-Campos, Y. Zhao, D.I. Stuart, G.C. Paesen, J.M. Grimes, A.A. Antson, O.W. Bayfield, D.E.D.P. Hawkins, D.-S. Ker, B. Wang, L. Turtle, K. Subramaniam, P. Thomson, P. Zhang, C. Dold, J. Ratcliff, P. Simmonds, T. de Silva, P. Sopp, D. Wellington, U. Rajapaksa, Y.-L. Chen, M. Salio, G. Napolitani, W. Paes, P. Borrow, B.M. Kessler, J.W. Fry, N.F. Schwabe, M.G. Semple, J.K. Baillie, S.C. Moore, P.J.M. Openshaw, M.A. Ansari, S. Dunachie, E. Barnes, J. Frater, G. Kerr, P. Goulder, T. Lockett, R. Levin, Y. Zhang, R. Jing, L.-P. Ho, E. Barnes, D. Dong, T. Dong, S. Dunachie, J. Frater, P. Goulder, G. Kerr, P. Klenerman, G. Liu, A. McMichael, G. Napolitani, G. Ogg, Y. Peng, M. Salio, X. Yao, Z. Yin, J. Kenneth Baillie, P. Klenerman, A.J. Mentzer, S.C. Moore, P.J.M. Openshaw, M.G. Semple, D.I. Stuart, L. Turtle, R.J. Cornall, C.P. Conlon, P. Klenerman, G.R. Screaton, J. Mongkolsapaya, A. McMichael, J.C. Knight, G. Ogg, T. Dong, T.c.C. Oxford Immunology Network Covid-19 Response, and I.C. Investigators, Broad and strong memory CD4+ and CD8+ T cells induced by SARS-CoV-2 in UK convalescent individuals following COVID-19. Nature Immunology 21 (2020) 1336-1345.

A. Grifoni, D. Weiskopf, S.I. Ramirez, J. Mateus, J.M. Dan, C.R. Moderbacher, S.A. Rawlings, A. Sutherland, L. Premkumar, R.S. Jadi, D. Marrama, A.M. de Silva, A. Frazier, A.F. Carlin, J.A. Greenbaum, B. Peters, F. Krammer, D.M. Smith, S. Crotty, and A. Sette, Targets of T Cell Responses to SARS-CoV-2 Coronavirus in Humans with COVID-19 Disease and Unexposed Individuals. Cell 181 (2020) 1489-1501.e15.

D. Blanco-Melo, B.E. Nilsson-Payant, W.-C. Liu, R. Moller, M. Panis, D. Sachs, R.A. Albrecht, and B.R. tenOever, SARS-CoV-2 launches a unique transcriptional signature from in vitro, ex vivo, and in vivo systems. bioRxiv (2020) 2020.03.24.004655.

M. Hoffmann, P. Arora, R. Grol3, A. Seidel, B. Hornich, A. Hahn, N. Kruger, L. Graichen, H. Hofmann-Winkler, A. Kempf, M.S. Winkler, S. Schulz, H.-M. Jack, B. Jahrsdbrfer, H. Schrezenmeier, M. Muller, A. Kleger, J. Munch, and S. Pohlmann, SARS-CoV-2 variants B.1.351 and B.1.1.248: Escape from therapeutic antibodies and antibodies induced by infection and vaccination. bioRxiv (2021) 2021.02.11.430787.

J.P. Moore, and P.A. Offit, SARS-CoV-2 Vaccines and the Growing Threat of Viral Variants. Jama 325 (2021) 821-822.

P. Wang, M.S. Nair, L. Liu, S. Iketani, Y. Luo, Y. Guo, M. Wang, J. Yu, B. Zhang, P.D. Kwong, B.S. Graham, J.R. Mascola, J.Y. Chang, M.T. Yin, M. Sobieszczyk, C.A. Kyratsous, L. Shapiro, Z. Sheng, Y. Huang, and D.D. Ho, Antibody resistance of SARS-CoV-2 variants B.1.351 and B.1.1.7. Nature 593 (2021) 130-135.

D.A. Collier, A. De Marco, I.A.T.M. Ferreira, B. Meng, R.P. Datir, A.C. Walls, S.A. Kemp, J. Bassi, D. Pinto, C. Silacci-Fregni, S. Bianchi, M.A. Tortorici, J. Bowen, K. Culap, S. Jaconi, E. Cameroni, G. Snell, M.S. Pizzuto, A.F. Pellanda, C. Garzoni, A. Riva, S. Baker, G. Dougan, C. Hess, N. Kingston, P.J. Lehner, P.A. Lyons, N.J. Matheson, W.H. Owehand, C. Saunders, C. Summers, J.E.D. Thaventhiran, M. Toshner, M.P. Weekes, A. Bucke, J. Calder, L. Canna, J. Domingo, A. Elmer, S. Fuller, J. Harris, S. Hewitt, J. Kennet, S. Jose, J. Kourampa, A. Meadows, C. O′Brien, J. Price, C. Publico, R. Rastall, C. Ribeiro, J. Rowlands, V. Ruffolo, H. Tordesillas, B. Bullman, B.J. Dunmore, S. Fawke, S. Graf, J. Hodgson, C. Huang, K. Hunter, E. Jones, E. Legchenko, C. Matara, J. Martin, F. Mescia, C. O′Donnell, L. Pointon, N. Pond, J. Shih, R. Sutcliffe, T. Tilly, C. Treacy, Z. Tong, J. Wood, M. Wylot, L. Bergamaschi, A. Betancourt, G. Bower, C. Cossetti, A. De Sa, M. Epping, R. Grenfell, A. Hinch, O. Huhn, S. Jackson, I. Jarvis, D. Lewis, J. Marsden, F. Nice, G. Okecha, O. Omarjee, M. Perera, N. Richoz, V. Romashova, N.S. Yarkoni, R. Sharma, L. Stefanucci, J. Stephens, M. Strezlecki, et al., Sensitivity of SARS-CoV-2 B.1.1.7 to mRNA vaccine-elicited antibodies. Nature 593 (2021) 136-141.

J. Wise, Covid-19: The E484K mutation and the risks it poses. BMJ 372 (2021) n359.

R.N. Thompson, E.M. Hill, and J.R. Gog, SARS-CoV-2 incidence and vaccine escape. Lancet Infect Dis (2021).

D. The Lancet Infectious, An exceptional vaccination policy in exceptional circumstances. Lancet Infect Dis 21 (2021) 149-149.

ig.ft.com/coronavirus-vaccine-tracker/?areas=gbr&areas=isr&areas=usa&areas=eue&cumulative=1&populationAdj usted=1

O.J. Wouters, K.C. Shadlen, M. Salcher-Konrad, A.J. Pollard, H.J. Larson, Y. Teerawattananon, and M. Jit, Challenges in ensuring global access to COVID-19 vaccines: production, affordability, allocation, and deployment. Lancet 397 (2021) 1023-1034.

M.M. Erdman, K.I. Kamrud, D.L. Harris, and J. Smith, Alphavirus replicon particle vaccines developed for use in humans induce high levels of antibodies to influenza virus hemagglutinin in swine: proof of concept. Vaccine 28 (2010) 594-6.

M. Hevey, D. Negley, P. Pushko, J. Smith, and A. Schmaljohn, Marburg virus vaccines based upon alphavirus replicons protect guinea pigs and nonhuman primates. Virology 251 (1998) 28-37.

J.W. Hooper, A.M. Ferro, J.W. Golden, P. Silvera, J. Dudek, K. Alterson, M. Custer, B. Rivers, J. Morris, G. Owens, J.F. Smith, and K.I. Kamrud, Molecular smallpox vaccine delivered by alphavirus replicons elicits protective immunity in mice and non-human primates. Vaccine 28 (2009) 494-511.

D.I. Bernstein, E.A. Reap, K. Katen, A. Watson, K. Smith, P. Norberg, R.A. Olmsted, A. Hoeper, J. Morris, S. Negri, M.F. Maughan, and J.D. Chulay, Randomized, double-blind, Phase 1 trial of an alphavirus replicon vaccine for cytomegalovirus in CMV seronegative adult volunteers. Vaccine 28 (2009) 484-93.

F. Diaz-San Segundo, C.C.A. Dias, M.P. Moraes, M. Weiss, E. Perez-Martin, G. Owens, M. Custer, K. Kamrud, T. de los Santos, and M.J. Grubman, Venezuelan equine encephalitis replicon particles can induce rapid protection against foot-and-mouth disease virus. Journal of virology 87 (2013) 5447-5460.

D.S. Reed, P.J. Glass, R.R. Bakken, J.F. Barth, C.M. Lind, L. da Silva, M.K. Hart, J. Rayner, K. Alterson, M. Custer, J. Dudek, G. Owens, K.I. Kamrud, M.D. Parker, and J. Smith, Combined alphavirus replicon particle vaccine induces durable and cross-protective immune responses against equine encephalitis viruses. J Virol 88 (2014) 12077-86.

D. Deming, T. Sheahan, M. Heise, B. Yount, N. Davis, A. Sims, M. Suthar, J. Harkema, A. Whitmore, R. Pickles, A. West, E. Donaldson, K. Curtis, R. Johnston, and R. Baric, Vaccine efficacy in senescent mice challenged with recombinant SARS-CoV bearing epidemic and zoonotic spike variants. PLoS Med 3 (2006) e525.

P. Pushko, M. Bray, G.V. Ludwig, M. Parker, A. Schmaljohn, A. Sanchez, P.B. Jahrling, and J.F. Smith, Recombinant RNA replicons derived from attenuated Venezuelan equine encephalitis virus protect guinea pigs and mice from Ebola hemorrhagic fever virus. Vaccine 19 (2000) 142-53.

K. Lundstrom, Alphavirus-based vaccines. Viruses 6 (2014) 2392-415.

J.H. Strauss, and E.G. Strauss, The alphaviruses: gene expression, replication, and evolution. Microbiol Rev 58 (1994) 491-562.

D.J. Paterson, W.A. Jefferies, J.R. Green, M.R. Brandon, P. Corthesy, M. Puklavec, and A.F. Williams, Antigens of activated rat T lymphocytes including a molecule of 50,000 Mr detected only on CD4 positive T blasts. Mol Immunol 24 (1987) 1281-90.

D.M. Calderhead, J.E. Buhlmann, A.J. van den Eertwegh, E. Claassen, R.J. Noelle, and H.P. Fell, Cloning of mouse Ox40: a T cell activation marker that may mediate T-B cell interactions. The Journal of Immunology 151 (1993) 5261-5271.

M. Croft, Control of immunity by the TNFR-related molecule OX40 (CD 134). Annu Rev Immunol 28 (2010) 57-78.

R.A. Zander, N. Obeng-Adjei, J.J. Guthmiller, D.I. Kulu, J. Li, A. Ongoiba, B. Traore, P.D. Crompton, and N.S. Butler, PD-1 Co-inhibitory and OX40 Co-stimulatory Crosstalk Regulates Helper T Cell Differentiation and Anti-Plasmodium Humoral Immunity. Cell Host Microbe 17 (2015) 628-41.

Q. Wang, B.-M. Shi, F. Xie, Z.-y. Fu, Y.-J. Chen, J.-N. An, Y. Ma, C.-P. Liu, X.-K. Zhang, and X.-G. Zhang, Enhancement of CD4+ T cell response and survival via coexpressed OX40/OX40L in Graves’ disease. Molecular and Cellular Endocrinology 430 (2016) 115-124.

R.A. Zander, R. Vijay, A.D. Pack, J.J. Guthmiller, A.C. Graham, S.E. Lindner, A.M. Vaughan, S.H.I. Kappe, and N. S. Butler, Thl-like Plasmodium-Specific Memory CD4+ T Cells Support Humoral Immunity. Cell Reports 21 (2017) 1839-1852.

I. Gramaglia, A.D. Weinberg, M. Lemon, and M. Croft, Ox-40 ligand: a potent costimulatory molecule for sustaining primary CD4 T cell responses. J Immunol 161 (1998) 6510-7.

P. Bansal-Pakala, B.S. Halteman, M.H. Cheng, and M. Croft, Costimulation of CD8 T cell responses by OX40. J Immunol 172 (2004) 4821-5.

S.F. Mousavi, P. Soroosh, T. Takahashi, Y. Yoshikai, H. Shen, L. Lefrancois, J. Borst, K. Sugamura, and N. Ishii, OX40 costimulatory signals potentiate the memory commitment of effector CD8+ T cells. Journal of immunology (Baltimore, Md. : 1950) 181 (2008) 5990-6001.

V. Tahiliani, T.E. Hutchinson, G. Abboud, M. Croft, and S. Salek-Ardakani, OX40 Cooperates with ICOS To Amplify Follicular Th Cell Development and Germinal Center Reactions during Infection. J Immunol 198 (2017) 218-228.

G. Lindgren, S. Ols, F. Liang, E.A. Thompson, A. Lin, F. Hellgren, K. Bahl, S. John, O. Yuzhakov, K.J. Hassett, L.A. Brito, H. Salter, G. Ciaramella, and K. Lore, Induction of Robust B Cell Responses after Influenza mRNA Vaccination Is Accompanied by Circulating Hemagglutinin-Specific ICOS+ PD-1+ CXCR3+ T Follicular Helper Cells. Front Immunol 8 (2017) 1539.

A. Cardeno, M.K. Magnusson, M. Quiding-Jarbrink, and A. Lundgren, Activated T follicular helper-like cells are released into blood after oral vaccination and correlate with vaccine specific mucosal B-cell memory. Scientific reports 8 (2018) 2729-2729.

M.A. Linterman, and D.L. Hill, Can follicular helper T cells be targeted to improve vaccine efficacy? F1000Res 5 (2016) F1000 Faculty Rev-88.

J.M. Hardwick, and B. Levine, Sindbis virus vector system for functional analysis of apoptosis regulators. Methods Enzymol 322 (2000) 492-508.

T. Granot, Y. Yamanashi, and D. Meruelo, Sindbis viral vectors transiently deliver tumor-associated antigens to lymph nodes and elicit diversified antitumor CD8+ T-cell immunity. Molecular therapy : the journal of the American Society of Gene Therapy 22 (2014) 112-22.

I. Scherwitzl, A. Hurtado, C.M. Pierce, S. Vogt, C. Pampeno, and D. Meruelo, Systemically Administered Sindbis Virus in Combination with Immune Checkpoint Blockade Induces Curative Anti-tumor Immunity. Molecular therapy oncolytics 9 (2018) 51-63.

I. Scherwitzl, S. Opp, A.M. Hurtado, C. Pampeno, C. Loomis, K. Kannan, M. Yu, and D. Meruelo, Sindbis Virus with Anti-OX40 Overcomes the Immunosuppressive Tumor Microenvironment of Low-Immunogenic Tumors. Molecular therapy oncolytics 17 (2020) 431-447.

A.H.A. Scherwitzl I, Pierce CM, Vogt S, Pampeno C, and Meruelo D., Systemically administered Sindbis virus in combination with immune checkpoint blockade induces curative anti-tumor immunity. MolecularTherapy Oncolytics 9 (2018) 51-63.

M. Yu, I. Scherwitzl, S. Opp, A. Tsirigos, and D. Meruelo, Molecular and metabolic pathways mediating curative treatment of a non-Hodgkin B cell lymphoma by Sindbis viral vectors and anti-4-1BB monoclonal antibody. Journal for immunotherapy of cancer 7 (2019) 185.

L.A. Jackson, E.J. Anderson, N.G. Rouphael, P.C. Roberts, M. Makhene, R.N. Coler, M.P. McCullough, J.D. Chappell, M.R. Denison, L.J. Stevens, A.J. Pruijssers, A. McDermott, B. Flach, N.A. Doria-Rose, K.S. Corbett, K.M. Morabito, S. O′Dell, S.D. Schmidt, P.A. Swanson, 2nd, M. Padilla, J.R. Mascola, K.M. Neuzil, H. Bennett, W. Sun, E. Peters, M. Makowski, J. Albert, K. Cross, W. Buchanan, R. Pikaart-Tautges, J.E. Ledgerwood, B.S. Graham, and J.H. Beigel, An mRNA Vaccine against SARS-CoV-2 - Preliminary Report. N Engl J Med 383 (2020) 1920-1931.

K.S. Corbett, B. Flynn, K.E. Foulds, J.R. Francica, S. Boyoglu-Barnum, A.P. Werner, B. Flach, S. O′Connell, K.W. Bock, M. Minai, B.M. Nagata, H. Andersen, D.R. Martinez, A.T. Noe, N. Douek, M.M. Donaldson, N.N. Nji, G.S. Alvarado, D.K. Edwards, D.R. Flebbe, E. Lamb, N.A. Doria-Rose, B.C. Lin, M.K. Louder, S. O′Dell, S.D. Schmidt, E. Phung, L.A. Chang, C. Yap, J.M. Todd, L. Pessaint, A. Van Ry, S. Browne, J. Greenhouse, T. Putman-Taylor, A. Strasbaugh, T.A. Campbell, A. Cook, A. Dodson, K. Steingrebe, W. Shi, Y. Zhang, O.M. Abiona, L. Wang, A. Pegu, E.S. Yang, K. Leung, T. Zhou, I.T. Teng, A. Widge, I. Gordon, L. Novik, R.A. Gillespie, R.J. Loomis, J.I. Moliva, G. Stewart-Jones, S. Himansu, W.P. Kong, M.C. Nason, K.M. Morabito, T.J. Ruckwardt, J.E. Ledgerwood, M.R. Gaudinski, P.D. Kwong, J.R. Mascola, A. Carfi, M.G. Lewis, R.S. Baric, A. McDermott, I.N. Moore, N.J. Sullivan, M. Roederer, R.A. Seder, and B.S. Graham, Evaluation of the mRNA-1273 Vaccine against SARS-CoV-2 in Nonhuman Primates. N Engl J Med 383 (2020) 1544-1555.

U. Sahin, A. Muik, E. Derhovanessian, I. Vogler, L.M. Kranz, M. Vormehr, A. Baum, K. Pascal, J. Quandt, D. Maurus, S. Brachtendorf, V. Lorks, J. Sikorski, R. Hilker, D. Becker, A.-K. Eller, J. Grutzner, C. Boesler, C. Rosenbaum, M.-C. Kuhnle, U. Luxemburger, A. Kemmer-Bruck, D. Langer, M. Bexon, S. Bolte, K. Kariko, T. Palanche, B. Fischer, A. Schultz, P.-Y. Shi, C. Fontes-Garfias, J.L. Perez, K.A. Swanson, J. Loschko, I.L. Scully, M. Cutler, W. Kalina, C.A. Kyratsous, D. Cooper, P.R. Dormitzer, K.U. Jansen, and Ö. Türeci, COVID-19 vaccine BNT162b1 elicits human antibody and TH1 T cell responses. Nature 586 (2020) 594-599.

P. Zhou, X.-L. Yang, X.-G. Wang, B. Hu, L. Zhang, W. Zhang, H.-R. Si, Y. Zhu, B. Li, C.-L. Huang, H.-D. Chen, J. Chen, Y. Luo, H. Guo, R.-D. Jiang, M.-Q. Liu, Y. Chen, X.-R. Shen, X. Wang, X.-S. Zheng, K. Zhao, Q.-J. Chen, F. Deng, L.-L. Liu, B. Yan, F.-X. Zhan, Y.-Y. Wang, G.-F. Xiao, and Z.-L. Shi, A pneumonia outbreak associated with a new coronavirus of probable bat origin. Nature 579 (2020) 270-273.

S. Xia, M. Liu, C. Wang, W. Xu, Q. Lan, S. Feng, F. Qi, L. Bao, L. Du, S. Liu, C. Qin, F. Sun, Z. Shi, Y. Zhu, S. Jiang, and L. Lu, Inhibition of SARS-CoV-2 (previously 2019-nCoV) infection by a highly potent pan-coronavirus fusion inhibitor targeting its spike protein that harbors a high capacity to mediate membrane fusion. Cell Res 30 (2020) 343-355.

X. Ou, Y. Liu, X. Lei, P. Li, D. Mi, L. Ren, L. Guo, R. Guo, T. Chen, J. Hu, Z. Xiang, Z. Mu, X. Chen, J. Chen, K. Hu, Q. Jin, J. Wang, and Z. Qian, Characterization of spike glycoprotein of SARS-CoV-2 on virus entry and its immune cross-reactivity with SARS-CoV. Nat Commun 11 (2020) 1620.

S. Xia, L. Yan, W. Xu, A.S. Agrawal, A. Algaissi, C.K. Tseng, Q. Wang, L. Du, W. Tan, I.A. Wilson, S. Jiang, B. Yang, and L. Lu, A pan-coronavirus fusion inhibitor targeting the HR1 domain of human coronavirus spike. Sci Adv 5 (2019) eaav4580.

C. Gaebler, Z. Wang, J.C.C. Lorenzi, F. Muecksch, S. Finkin, M. Tokuyama, A. Cho, M. Jankovic, D. Schaefer-Babajew, T.Y. Oliveira, M. Cipolla, C. Viant, C.O. Barnes, Y. Bram, G. Breton, T. Hägglöf, P. Mendoza, A. Hurley, M. Turroja, K. Gordon, K.G. Millard, V. Ramos, F. Schmidt, Y. Weisblum, D. Jha, M. Tankelevich, G. Martinez-Delgado, J. Yee, R. Patel, J. Dizon, C. Unson-O′Brien, I. Shimeliovich, D.F. Robbiani, Z. Zhao, A. Gazumyan, R.E. Schwartz, T. Hatziioannou, P.J. Bjorkman, S. Mehandru, P.D. Bieniasz, M. Caskey, and M.C. Nussenzweig, Evolution of antibody immunity to SARS-CoV-2. Nature 591 (2021) 639-644.

D.F. Robbiani, C. Gaebler, F. Muecksch, J.C.C. Lorenzi, Z. Wang, A. Cho, M. Agudelo, C.O. Barnes, A. Gazumyan, S. Finkin, T. Hägglöf, T.Y. Oliveira, C. Viant, A. Hurley, H.-H. Hoffmann, K.G. Millard, R.G. Kost, M. Cipolla, K. Gordon, F. Bianchini, S.T. Chen, V. Ramos, R. Patel, J. Dizon, I. Shimeliovich, P. Mendoza, H. Hartweger, L. Nogueira, M. Pack, J. Horowitz, F. Schmidt, Y. Weisblum, E. Michailidis, A.W. Ashbrook, E. Waltari, J.E. Pak, K.E. Huey-Tubman, N. Koranda, P.R. Hoffman, A.P. West, C.M. Rice, T. Hatziioannou, P.J. Bjorkman, P.D. Bieniasz, M. Caskey, and M.C. Nussenzweig, Convergent antibody responses to SARS-CoV-2 in convalescent individuals. Nature 584 (2020) 437-442.

F. Schmidt, Y. Weisblum, F. Muecksch, H.H. Hoffmann, E. Michailidis, J.C.C. Lorenzi, P. Mendoza, M. Rutkowska, E. Bednarski, C. Gaebler, M. Agudelo, A. Cho, Z. Wang, A. Gazumyan, M. Cipolla, M. Caskey, D.F. Robbiani, M.C. Nussenzweig, C.M. Rice, T. Hatziioannou, and P.D. Bieniasz, Measuring SARS-CoV-2 neutralizing antibody activity using pseudotyped and chimeric viruses. The Journal of experimental medicine 217 (2020).

L. Bao, W. Deng, B. Huang, H. Gao, J. Liu, L. Ren, Q. Wei, P. Yu, Y. Xu, F. Qi, Y. Qu, F. Li, Q. Lv, W. Wang, J. Xue, S. Gong, M. Liu, G. Wang, S. Wang, Z. Song, L. Zhao, P. Liu, L. Zhao, F. Ye, H. Wang, W. Zhou, N. Zhu, W. Zhen, H. Yu, X. Zhang, L. Guo, L. Chen, C. Wang, Y. Wang, X. Wang, Y. Xiao, Q. Sun, H. Liu, F. Zhu, C. Ma, L. Yan, M. Yang, J. Han, W. Xu, W. Tan, X. Peng, Q. Jin, G. Wu, and C. Qin, The pathogenicity of SARS-CoV-2 in hACE2 transgenic mice. Nature 583 (2020) 830-833.

C. Rydyznski Moderbacher, S.I. Ramirez, J.M. Dan, A. Grifoni, K.M. Hastie, D. Weiskopf, S. Belanger, R.K. Abbott, C. Kim, J. Choi, Y. Kato, E.G. Crotty, C. Kim, S.A. Rawlings, J. Mateus, L.P.V. Tse, A. Frazier, R. Baric, B. Peters, J. Greenbaum, E. Ollmann Saphire, D.M. Smith, A. Sette, and S. Crotty, Antigen-Specific Adaptive Immunity to SARS-CoV-2 in Acute COVID-19 and Associations with Age and Disease Severity. Cell 183 (2020) 996-1012.e19.

G.J. van der Windt, B. Everts, C.H. Chang, J.D. Curtis, T.C. Freitas, E. Amiel, E.J. Pearce, and E.L. Pearce, Mitochondrial respiratory capacity is a critical regulator of CD8+ T cell memory development. Immunity 36 (2012) 68-78.

S. Li, N.L. Sullivan, N. Rouphael, T. Yu, S. Banton, M.S. Maddur, M. McCausland, C. Chiu, J. Canniff, S. Dubey, K. Liu, V. Tran, T. Hagan, S. Duraisingham, A. Wieland, A.K. Mehta, J.A. Whitaker, S. Subramaniam, D.P. Jones, A. Sette, K. Vora, A. Weinberg, M.J. Mulligan, H.I. Nakaya, M. Levin, R. Ahmed, and B. Pulendran, Metabolic Phenotypes of Response to Vaccination in Humans. Cell 169 (2017) 862-877.e17.

A.V. Menk, N.E. Scharping, R.S. Moreci, X. Zeng, C. Guy, S. Salvatore, H. Bae, J. Xie, H.A. Young, S.G. Wendell, and G.M. Delgoffe, Early TCR Signaling Induces Rapid Aerobic Glycolysis Enabling Distinct Acute T Cell Effector Functions. Cell Rep 22 (2018) 1509-1521.

N. Jones, J.G. Cronin, G. Dolton, S. Panetti, A.J. Schauenburg, S.A.E. Galloway, A.K. Sewell, D.K. Cole, C.A. Thornton, and N.J. Francis, Metabolic Adaptation of Human CD4+ and CD8+ T-Cells to T-Cell Receptor-Mediated Stimulation. Frontiers in Immunology 8 (2017).

J.E. McElhaney, D. Xie, W.D. Hager, M.B. Barry, Y. Wang, A. Kleppinger, C. Ewen, K.P. Kane, and R.C. Bleackley, T cell responses are better correlates of vaccine protection in the elderly. J Immunol 176 (2006) 6333-9.

J.E. McElhaney, C. Ewen, X. Zhou, K.P. Kane, D. Xie, W.D. Hager, M.B. Barry, A. Kleppinger, Y. Wang, and R.C. Bleackley, Granzyme B: Correlates with protection and enhanced CTL response to influenza vaccination in older adults. Vaccine 27 (2009) 2418-25.

K. Hashimoto, T. Kouno, T. Ikawa, N. Hayatsu, Y. Miyajima, H. Yabukami, T. Terooatea, T. Sasaki, T. Suzuki, M. Valentine, G. Pascarella, Y. Okazaki, H. Suzuki, J.W. Shin, A. Minoda, I. Taniuchi, H. Okano, Y. Arai, N. Hirose, and P. Carninci, Single-cell transcriptomics reveals expansion of cytotoxic CD4 T cells in supercentenarians. Proceedings of the National Academy of Sciences 116 (2019) 24242-24251.

B.S. Graham, Rapid COVID-19 vaccine development. Science (New York, N.Y.) 368 (2020) 945-946.

M.J. Mulligan, K.E. Lyke, N. Kitchin, J. Absalon, A. Gurtman, S. Lockhart, K. Neuzil, V. Raabe, R. Bailey, K.A. Swanson, P. Li, K. Koury, W. Kalina, D. Cooper, C. Fontes-Garfias, P.Y. Shi, Ö. Türeci, K.R. Tompkins, E.E. Walsh, R. Frenck, A.R. Falsey, P.R. Dormitzer, W.C. Gruber, U. Sahin, and K.U. Jansen, Phase I/II study of COVID-19 RNA vaccine BNT162b1 in adults. Nature 586 (2020) 589-593.

F.C. Zhu, X.H. Guan, Y.H. Li, J.Y. Huang, T. Jiang, L.H. Hou, J.X. Li, B.F. Yang, L. Wang, W.J. Wang, S.P. Wu, Z. Wang, X.H. Wu, J.J. Xu, Z. Zhang, S.Y. Jia, B.S. Wang, Y. Hu, J.J. Liu, J. Zhang, X.A. Qian, Q. Li, H.X. Pan, H.D. Jiang, P. Deng, J.B. Gou, X.W. Wang, X.H. Wang, and W. Chen, Immunogenicity and safety of a recombinant adenovirus type-5-vectored COVID-19 vaccine in healthy adults aged 18 years or older: a randomised, double-blind, placebo-controlled, phase 2 trial. Lancet 396 (2020) 479-488.

P.M. Folegatti, K.J. Ewer, P.K. Aley, B. Angus, S. Becker, S. Belij-Rammerstorfer, D. Bellamy, S. Bibi, M. Bittaye, E.A. Clutterbuck, C. Dold, S.N. Faust, A. Finn, A.L. Flaxman, B. Hallis, P. Heath, D. Jenkin, R. Lazarus, R. Makinson, A.M. Minassian, K.M. Pollock, M. Ramasamy, H. Robinson, M. Snape, R. Tarrant, M. Voysey, C. Green, A.D. Douglas, A.V.S. Hill, T. Lambe, S.C. Gilbert, and A.J. Pollard, Safety and immunogenicity of the ChAdOx1 nCoV-19 vaccine against SARS-CoV-2: a preliminary report of a phase ½, single-blind, randomised controlled trial. Lancet 396 (2020) 467-478.

J.H. Tian, N. Patel, R. Haupt, H. Zhou, S. Weston, H. Hammond, J. Logue, A.D. Portnoff, J. Norton, M. Guebre-Xabier, B. Zhou, K. Jacobson, S. Maciejewski, R. Khatoon, M. Wisniewska, W. Moffitt, S. Kluepfel-Stahl, B. Ekechukwu, J. Papin, S. Boddapati, C. Jason Wong, P.A. Piedra, M.B. Frieman, M.J. Massare, L. Fries, K.L. Bengtsson, L. Stertman, L. Ellingsworth, G. Glenn, and G. Smith, SARS-CoV-2 spike glycoprotein vaccine candidate NVX-CoV2373 immunogenicity in baboons and protection in mice. Nat Commun 12 (2021) 372.

M.N. Fleeton, M. Chen, P. Berglund, G. Rhodes, S.E. Parker, M. Murphy, G.J. Atkins, and P. Liljeström, Self-replicative RNA vaccines elicit protection against influenza A virus, respiratory syncytial virus, and a tickborne encephalitis virus. J Infect Dis 183 (2001) 1395-8.

C. Andersson, P. Liljestrom, S. Ståhl, and U.F. Power, Protection against respiratory syncytial virus (RSV) elicited in mice by plasmid DNA immunisation encoding a secreted RSV G protein-derived antigen. FEMS Immunol Med Microbiol 29 (2000) 247-53.

P. Roques, K. Ljungberg, B.M. Kummerer, L. Gosse, N. Dereuddre-Bosquet, N. Tchitchek, D. Hallengärd, J. García-Arriaza, A. Meinke, M. Esteban, A. Merits, R. Le Grand, and P. Liljeström, Attenuated and vectored vaccines protect nonhuman primates against Chikungunya virus. JCI Insight 2 (2017) e83527.

N. Wressnigg, R. Hochreiter, O. Zoihsl, A. Fritzer, N. Bezay, A. Klingler, K. Lingnau, M. Schneider, U. Lundberg, A. Meinke, J. Larcher-Senn, I. Corbic-Ramljak, S. Eder-Lingelbach, K. Dubischar, and W. Bender, Single-shot live-attenuated chikungunya vaccine in healthy adults: a phase 1, randomised controlled trial. The Lancet Infectious Diseases 20 (2020) 1193-1203.

S. van de Wall, K. Ljungberg, P.P. Ip, A. Boerma, M.L. Knudsen, H.W. Nijman, P. Liljestrom, and T. Daemen, Potent therapeutic efficacy of an alphavirus replicon DNA vaccine expressing human papilloma virus E6 and E7 antigens. Oncoimmunology 7 (2018) e1487913-e1487913.

K. Lundstrom, Alphavirus vectors for vaccine production and gene therapy. Expert Review of Vaccines 2 (2003) 445-459.

S.A. Plotkin, Correlates of protection induced by vaccination. Clin Vaccine Immunol 17 (2010) 1055-1065.

K.J. Ewer, J.R. Barrett, S. Belij-Rammerstorfer, H. Sharpe, R. Makinson, R. Morter, A. Flaxman, D. Wright, D. Bellamy, M. Bittaye, C. Dold, N.M. Provine, J. Aboagye, J. Fowler, S.E. Silk, J. Alderson, P.K. Aley, B. Angus, E. Berrie, S. Bibi, P. Cicconi, E.A. Clutterbuck, I. Chelysheva, P.M. Folegatti, M. Fuskova, C.M. Green, D. Jenkin, S. Kerridge, A. Lawrie, A.M. Minassian, M. Moore, Y. Mujadidi, E. Plested, I. Poulton, M.N. Ramasamy, H. Robinson, R. Song, M.D. Snape, R. Tarrant, M. Voysey, M.E.E. Watson, A.D. Douglas, A.V.S. Hill, S.C. Gilbert, A.J. Pollard, T. Lambe, A. Ali, E. Allen, M. Baker, E. Barnes, N. Borthwick, A. Boyd, C. Brown-O′Sullivan, J. Burgoyne, N. Byard, I.C. Puig, F. Cappuccini, J.-S. Cho, P. Cicconi, E. Clark, W.E.M. Crocker, M.S. Datoo, H. Davies, S.J. Dunachie, N.J. Edwards, S.C. Elias, J. Furze, C. Gilbride, S.A. Harris, S.H.C. Hodgson, M.M. Hou, S. Jackson, K. Jones, R. Kailath, L. King, C.W. Larkworthy, Y. Li, A.M. Lias, A. Linder, S. Lipworth, R.L. Ramon, M. Madhavan, E. Marlow, J.L. Marshall, A.J. Mentzer, H. Morrison, A. Noe, D. Pipini, D. Pulido-Gomez, F.R. Lopez, A.J. Ritchie, I. Rudiansyah, H. Sanders, A. Shea, S. Silk, A.J. Spencer, R. Tanner, Y. Themistocleous, M. Thomas, N. Tran, et al., T cell and antibody responses induced by a single dose of ChAdOx1 nCoV-19 (AZD1222) vaccine in a phase ½ clinical trial. Nature Medicine 27 (2021) 270-278.

A. Song, X. Tang, K.M. Harms, and M. Croft, OX40 and Bcl-x<sub>L</sub> Promote the Persistence of CD8 T Cells to Recall Tumor-Associated Antigen. The Journal of Immunology 175 (2005) 3534-3541.

D.A.G. Skibinski, L.A. Jones, Y.O. Zhu, L.W. Xue, B. Au, B. Lee, A.N.M. Naim, A. Lee, N. Kaliaperumal, J.G.H. Low, L.S. Lee, M. Poidinger, P. Saudan, M. Bachmann, E.E. Ooi, B.J. Hanson, V. Novotny-Diermayr, A. Matter, A.-M. Fairhurst, M.L. Hibberd, and J.E. Connolly, Induction of Human T-cell and Cytokine Responses Following Vaccination with a Novel Influenza Vaccine. Scientific Reports 8 (2018) 18007.

N. Otani, M. Shima, T. Ueda, K. Ichiki, K. Nakajima, Y. Takesue, and T. Okuno, Evaluation of influenza vaccine-immunogenicity in cell-mediated immunity. Cellular Immunology 310 (2016) 165-169.

N. van Doremalen, T. Lambe, A. Spencer, S. Belij-Rammerstorfer, J.N. Purushotham, J.R. Port, V.A. Avanzato, T. Bushmaker, A. Flaxman, M. Ulaszewska, F. Feldmann, E.R. Allen, H. Sharpe, J. Schulz, M. Holbrook, A. Okumura, K. Meade-White, L. Perez-Perez, N.J. Edwards, D. Wright, C. Bissett, C. Gilbride, B.N. Williamson, R. Rosenke, D. Long, A. Ishwarbhai, R. Kailath, L. Rose, S. Morris, C. Powers, J. Lovaglio, P.W. Hanley, D. Scott, G. Saturday, E. de Wit, S.C. Gilbert, and V.J. Munster, ChAdOx1 nCoV-19 vaccine prevents SARS-CoV-2 pneumonia in rhesus macaques. Nature 586 (2020) 578-582.

A. Chandrashekar, J. Liu, A.J. Martinot, K. McMahan, N.B. Mercado, L. Peter, L.H. Tostanoski, J. Yu, Z. Maliga, M. Nekorchuk, K. Busman-Sahay, M. Terry, L.M. Wrijil, S. Ducat, D.R. Martinez, C. Atyeo, S. Fischinger, J.S. Burke, M.D. Slein, L. Pessaint, A. Van Ry, J. Greenhouse, T. Taylor, K. Blade, A. Cook, B. Finneyfrock, R. Brown, E. Teow, J. Velasco, R. Zahn, F. Wegmann, P. Abbink, E.A. Bondzie, G. Dagotto, M.S. Gebre, X. He, C. Jacob-Dolan, N. Kordana, Z. Li, M.A. Lifton, S.H. Mahrokhian, L.F. Maxfield, R. Nityanandam, J.P. Nkolola, A.G. Schmidt, A.D. Miller, R.S. Baric, G. Alter, P.K. Sorger, J.D. Estes, H. Andersen, M.G. Lewis, and D.H. Barouch, SARS-CoV-2 infection protects against rechallenge in rhesus macaques. Science (New York, N.Y.) 369 (2020) 812-817.

T. Sekine, A. Perez-Potti, O. Rivera-Ballesteros, K. Strålin, J.B. Gorin, A. Olsson, S. Llewellyn-Lacey, H. Kamal, G. Bogdanovic, S. Muschiol, D.J. Wullimann, T. Kammann, J. Emgård, T. Parrot, E. Folkesson, O. Rooyackers, L.I. Eriksson, J.I. Henter, A. Sönnerborg, T. Allander, J. Albert, M. Nielsen, J. Klingström, S. Gredmark-Russ, N.K. Björkström, J.K. Sandberg, D.A. Price, H.G. Ljunggren, S. Aleman, and M. Buggert, Robust T Cell Immunity in Convalescent Individuals with Asymptomatic or Mild COVID-19. Cell 183 (2020) 158-168.e14.

D. Weiskopf, K.S. Schmitz, M.P. Raadsen, A. Grifoni, N.M.A. Okba, H. Endeman, J.P.C. van den Akker, R. Molenkamp, M.P.G. Koopmans, E.C.M. van Gorp, B.L. Haagmans, R.L. de Swart, A. Sette, and R.D. de Vries, Phenotype and kinetics of SARS-CoV-2-specific T cells in COVID-19 patients with acute respiratory distress syndrome. Science immunology 5 (2020) eabd2071.

K. Ljungberg, and P. Liljeström, Self-replicating alphavirus RNA vaccines. Expert Rev Vaccines 14 (2015) 177-94.

J.C. Tseng, A. Hurtado, H. Yee, B. Levin, C. Boivin, M. Benet, S.V. Blank, A. Pellicer, and D. Meruelo, Using sindbis viral vectors for specific detection and suppression of advanced ovarian cancer in animal models. Cancer research 64 (2004) 6684-92.

T. Granot, L. Venticinque, J.C. Tseng, and D. Meruelo, Activation of cytotoxic and regulatory functions of NK cells by Sindbis viral vectors. PloS one 6 (2011) e20598.

J.C. Tseng, B. Levin, T. Hirano, H. Yee, C. Pampeno, and D. Meruelo, In vivo antitumor activity of Sindbis viral vectors. Journal of the National Cancer Institute 94 (2002) 1790-802.

P. Bell, M. Limberis, G. Gao, D. Wu, M.S. Bove, J.C. Sanmiguel, and J.M. Wilson, An optimized protocol for detection of E. coli β-galactosidase in lung tissue following gene transfer. Histochemistry and Cell Biology 124 (2005) 77-85.

L. Lu, Q. Liu, Y. Zhu, K.H. Chan, L. Qin, Y. Li, Q. Wang, J.F. Chan, L. Du, F. Yu, C. Ma, S. Ye, K.Y. Yuen, R. Zhang, and S. Jiang, Structure-based discovery of Middle East respiratory syndrome coronavirus fusion inhibitor. Nat Commun 5 (2014) 3067.

A. Scaglione, J. Patzig, J. Liang, R. Frawley, J. Bok, A. Mela, C. Yattah, J. Zhang, S.X. Teo, T. Zhou, S. Chen, E. Bernstein, P. Canoll, E. Guccione, and P. Casaccia, PRMT5-mediated regulation of developmental myelination. Nat Commun 9 (2018) 2840.

A. Dobin, C.A. Davis, F. Schlesinger, J. Drenkow, C. Zaleski, S. Jha, P. Batut, M. Chaisson, and T.R. Gingeras, STAR: ultrafast universal RNA-seq aligner. Bioinformatics (Oxford, England) 29 (2013) 15-21.

Y. Liao, G.K. Smyth, and W. Shi, The Subread aligner: fast, accurate and scalable read mapping by seed-and-vote. Nucleic Acids Res 41 (2013) e108.

M.I. Love, W. Huber, and S. Anders, Moderated estimation of fold change and dispersion for RNA-seq data with DESeq2. Genome biology 15 (2014) 550.

A. Subramanian, P. Tamayo, V.K. Mootha, S. Mukherjee, B.L. Ebert, M.A. Gillette, A. Paulovich, S.L. Pomeroy, T.R. Golub, E.S. Lander, and J.P. Mesirov, Gene set enrichment analysis: a knowledge-based approach for interpreting genome-wide expression profiles. Proc Natl Acad Sci U S A 102 (2005) 15545-50.

V.K. Mootha, C.M. Lindgren, K.-F. Eriksson, A. Subramanian, S. Sihag, J. Lehar, P. Puigserver, E. Carlsson, M. Ridderstråle, E. Laurila, N. Houstis, M.J. Daly, N. Patterson, J.P. Mesirov, T.R. Golub, P. Tamayo, B. Spiegelman, E.S. Lander, J.N. Hirschhorn, D. Altshuler, and L.C. Groop, PGC-1α-responsive genes involved in oxidative phosphorylation are coordinately downregulated in human diabetes. Nature genetics 34 (2003) 267-273.

D. Szklarczyk, A.L. Gable, D. Lyon, A. Junge, S. Wyder, J. Huerta-Cepas, M. Simonovic, N.T. Doncheva, J.H. Morris, P. Bork, L.J. Jensen, and C.v. Mering, STRING v11: protein-protein association networks with increased coverage, supporting functional discovery in genome-wide experimental datasets. Nucleic Acids Res 47 (2019) D607-D613.

N.E. Scharping, A.V. Menk, R.S. Moreci, R.D. Whetstone, R.E. Dadey, S.C. Watkins, R.L. Ferris, and G.M. Delgoffe, The Tumor Microenvironment Represses T Cell Mitochondrial Biogenesis to Drive Intratumoral T Cell Metabolic Insufficiency and Dysfunction. Immunity 45 (2016) 701-703.

While the disclosure has been particularly shown and described with reference to specific embodiments, it should be understood by those having skill in the art that various changes in form and detail may be made therein without departing from the spirit and scope of the present disclosure as disclosed herein.

1. A modified Alphavirus encoding a SARS-CoV-2 spike protein or antigenic segment of the SARS-CoV-2 spike protein.

2. The modified Alphavirus of claim 1, wherein said modified Alphavirus is a replicative defective Sindbis virus.

3. The replicative defective Sindbis virus of claim 2, wherein the virus encodes one or more additional heterologous polypeptides comprising at least one immunomodulatory protein.

4. The replicative defective Sindbis virus of claim 3, wherein the at least one immunomodulatory protein is an anti-OX40 antibody or OX40 binding fragment thereof, an interleukin, or a combination thereof.

5. The replicative defective Sindbis virus of claim 4, encoding the anti-OX40 antibody.

6. The replicative defective Sindbis virus of claim 4, encoding the interleukin.

7. The replicative defective Sindbis virus of claim 4, encoding the anti-OX40 antibody and the interleukin.

8. The replicative defective Sindbis virus of claim 7, wherein the interleukin is interleukin 12 (IL-12).

9. A plurality of isolated replicative defective Sindbis viruses according to claim 1.

10. A pharmaceutical formulation comprising isolated replicative defective Sindbis viruses of claim 9.

11. The pharmaceutical formulation of claim 10 comprised by an inhalable formulation.

12. A method for prophylaxis or treatment for a SARS-CoV2 infection comprising administering to an individual a composition of claim 10.

13. The method of claim 12, wherein the at least one immunomodulatory protein comprises an anti-OX40 antibody or OX40 binding fragment thereof, an interleukin, or a combination thereof.

14. The method of claim 13, wherein the at least one immunomodulatory protein comprises the interleukin.

15. The method of claim 14, wherein the interleukin is interleukin 12 (IL-12).

16. The method of claim 15, further comprising administering to the individual an anti-OX40 antibody or OX40 binding fragment thereof, and wherein the anti-OX40 antibody or OX40 binding fragment thereof is not encoded by the replicative defective Sindbis virus.

17. The method of claim 15, wherein administering the replicative defective Sindbis viruses stimulates an immune response that prevents the individual from developing COVID-19 when exposed to SARS-CoV-2 and/or prevents infection by SARS-CoV-2 when exposed to SARS-CoV-2.

18. The method of claim 16, wherein administering the replicative defective Sindbis viruses stimulates an immune response that prevents the individual from developing COVID-19 when exposed to SARS-CoV-2 and/or prevents infection by SARS-CoV-2 when exposed to SARS-CoV-2.

19. A method of making replicative defective Sindbis viruses encoding a SARS-CoV-2 spike protein or an antigenic segment of the SARS-CoV-2 spike protein, the method comprising expressing in mammalian cells:
i) a first polynucleotide comprising a Sindbis genomic replicon encoding the SARS-CoV-2 spike protein or antigenic fragment thereof, said replicon further comprising Sindbis replicase genes nsP1, nsP2, nsP3 and nsP4, and including a functional packaging signal; and
ii) a second polynucleotide encoding Sindbis structural capsid proteins C, E1, E2, E3, and 6K, and lacking a functional packaging signal;
iii) allowing expression of the first and second polynucleotides within the mammalian cells; and
iv) separating replicative defective Sindbis viruses from the mammalian cells.

20. The method of claim 19, wherein the first polynucleotide further encodes one or more additional heterologous polypeptides.

21. The method of claim 19, wherein the i) further encodes one or more additional heterologous polypeptides.

22. The method of claim 21, wherein the one or more additional heterologous polypeptides comprise at least one immunomodulatory protein.

23. The method of claim 22, wherein at least one immunomodulatory protein is an anti-OX40 antibody or OX40 binding fragment thereof, an interleukin, or a combination thereof.


SEQUENCE LISTING (5 sequences)

SEQ ID NO: 1 - protein, 1286 aa, SARS CoV-2
GNATMFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 474 aa, artificial sequence
  Anti-OX40 IgG2a Heavy Chain
MGQSRYLLFLATLALLNHLSLAMAEVQLVESGGGLVQPGGSLRLSCAASGFTFSNYTMNWVRQAPGKGLEWVSAISGSGGSTYYADSVKGRFTISRDNSKNTLYLQMNSLRAEDTAVYYCAKDRYSQVHYALDYWGQGTLVTVAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGLTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRASQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 262 aa, artificial sequence
  Anti-OX40 IgG2a light chain
MGQSRYLLFLATLALLNHLSLADIQMTQSPDSLPVTPGEPASISCRSSQSLLHSNGYNYLDWYLQKAGQSPQLLIYLGSNRASGVPDRFSGSGSGTDFTLKISRVEAEDVGVYYCQQYYNHPTTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGSGEGRGSLLTCGDVEENPGP

SEQ ID NO: 4 - protein, 560 aa, Homo sapiens
MGQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAVPGVGVPGVGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSGEGRGSLLTCGDVEENPG

SEQ ID NO: 5 - DNA, 17840 bp, artificial sequence
  Sindbis Vector DNA sequence encoding viral proteins
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatatgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagaccatcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagctcactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcgaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggaccccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccacctctagcgtttaaacatgggccagagcaggtacctgctgttcctggccaccctggccctgctgaaccacctgagcctggccatggccgaggtgcagctggtggagagcggcggcggcctggtgcagcccggcggcagcctgaggctgagctgcgccgccagcggcttcaccttcagcaactacaccatgaactgggtgaggcaggcccccggcaagggcctggagtgggtgagcgccatcagcggcagcggcggcagcacctactacgccgacagcgtgaagggcaggttcaccatcagcagggacaacagcaagaacaccctgtacctgcagatgaacagcctgagggccgaggacaccgccgtgtactactgcgccaaggacaggtacagccaggtgcactacgccctggactactggggccagggcaccctggtgaccgtggccgccaagaccaccgcccccagcgtgtaccccctggcccccgtgtgcggcgacaccaccggcagcagcgtgaccctgggctgcctggtgaagggctacttccccgagcccgtgaccctgacctggaacagcggcagcctgagcagcggcgtgcacaccttccccgccgtgctgcagagcgacctgtacaccctgagcagcagcgtgaccgtgaccagcagcacctggcccagccagagcatcacctgcaacgtggcccaccccgccagcagcaccaaggtggacaagaagatcgagcccaggggcctcaccatcaagccctgccccccctgcaagtgccccgcccccaacctgctgggcggccccagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccacagggaggactacaacagcaccctgagggtggtgagcgccctgcccatccagcaccaggactggatgagcggcaaggagttcaagtgcaaggtgaacaacaaggacctgcccgcccccatcgagaggaccatcagcaagcccaagggcagcgtgagggcctcccaggtgtacgtgctgcccccccccgaggaggagatgaccaagaagcaggtgaccctgacctgcatggtgaccgacttcatgcccgaggacatctacgtggagtggaccaacaacggcaagaccgagctgaactacaagaacaccgagcccgtgctggacagcgacggcagctacttcatgtacagcaagctgagggtggagaagaagaactgggtggagaggaacagctacagctgcagcgtggtgcacgagggcctgcacaaccaccacaccaccaagagcttcagcaggacccccggcaagtaaggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccacctctagcgtttgtttgccaccatgggccagtcccggtacctgctgttcctggccaccctggccctgctgaaccacctgtccctggccgacatccagatgacccagtcccccgactccctgcccgtgacccccggcgagcccgcctccatctcctgccggtcctcccagtccctgctgcactccaacggctacaactacctggactggtacctgcagaaggccggccagtccccccagctgctgatctacctgggctccaaccgggcctccggcgtgcccgaccggttctccggctccggctccggcaccgacttcaccctgaagatctcccgggtggaggccgaggacgtgggcgtgtactactgccagcagtactacaaccaccccaccaccttcggccagggcaccaagctggagatcaagcgggccgacgccgcccccaccgtgtccatcttccccccctcctccgagcagctgacctccggcggcgcctccgtggtgtgcttcctgaacaacttctaccccaaggacatcaacgtgaagtggaagatcgacggctccgagcggcagaacggcgtgctgaactcctggaccgaccaggactccaaggactccacctactccatgtcctccaccctgaccctgaccaaggacgagtacgagcggcacaactcctacacctgcgaggccacccacaagacctccacctcccccatcgtgaagtccttcaaccggaacgagtgcggcagcggagagggcagaggctccctgctgacctgtggcgacgtggaagagaaccctggcccaatgggtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcatcctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccgttcctggagtaggggtacctggagtgggcggatctatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctggatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcagcggagagggcagaggctccctgctgacctgtggcgacgtggaagagaaccctggcccaggaaacgccaccatgttcctgctgaccacaaagcggacaatgttcgtgtttctggtgctgctgcctctggtgagctcccagtgcgtgaacctgaccacaagaacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagtgctgccctttaacgatggcgtgtacttcgcctccaccgagaagtctaacatcatccgcggctggatctttggcaccacactggacagcaagacacagtccctgctgatcgtgaacaatgccaccaacgtggtgatcaaggtgtgcgagttccagttttgtaatgatccattcctgggcgtgtactatcacaagaacaataagtcttggatggagagcgagtttcgggtgtattcctctgccaacaattgcacatttgagtacgtgtcccagcccttcctgatggacctggagggcaagcagggcaatttcaagaacctgcgggagttcgtgtttaagaatatcgatggctacttcaagatctactccaagcacaccccaatcaacctggtgagagacctgccacagggattctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagccccggaccttcctgctgaagtacaacgagaatggcaccatcacagacgcagtggattgcgccctggaccccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgccaccagattcgccagcgtgtacgcctggaataggaagcgcatctccaactgcgtggccgactattctgtgctgtacaacagcgcctccttctctacctttaagtgctatggcgtgagccccacaaagctgaatgatctgtgctttaccaacgtgtacgccgattccttcgtgatcaggggagacgaagtgaggcagatcgcaccaggacagacaggaaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagcgggacatctccacagagatctaccaggccggctctaccccctgcaatggcgtggagggctttaactgttatttccctctgcagagctacggcttccagccaaccaacggcgtgggctatcagccctacagagtggtggtgctgtcttttgagctgctgcacgcacctgcaacagtgtgcggcccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggacatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagcagagcacgtgaacaatagctatgagtgcgacatccctatcggcgccggcatctgtgcctcctaccagacccagacaaactccccaaggagagccaggtctgtggccagccagtccatcatcgcctataccatgagcctgggcgccgagaacagcgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatcgcgccctgacaggaatcgcagtggagcaggacaagaacacacaggaggtgttcgcccaggtgaagcagatctacaagaccccacccatcaaggactttggcggcttcaacttcagccagatcctgcccgatcctagcaagccatccaagaggtcttttatcgaggacctgctgttcaacaaggtgaccctggccgatgccggcttcatcaagcagtatggagattgcctgggagacatcgcagcccgcgacctgatctgtgcccagaagtttaatggcctgaccgtgctgcctccactgctgacagatgagatgatcgcccagtacacatctgccctgctggccggcaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggaaagctgcaggatgtggtgaatcagaacgcccaggccctgaataccctggtgaagcagctgtctagcaacttcggcgccatctcctctgtgctgaatgacatcctgagccggctggacaaggtggaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggacagtccaagagggtggacttttgtggcaagggctatcacctgatgagcttcccacagtccgcccctcacggcgtggtgtttctgcacgtgacctacgtgccagcccaggagaagaacttcaccacagcaccagccatctgccacgatggaaaggcacactttcctagggagggcgtgttcgtgagcaacggcacccactggtttgtgacacagcgcaatttctacgagccacagatcatcaccacagacaataccttcgtgagcggcaactgtgatgtggtgatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacagactgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgagggcccaatgttccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaaatttaaattaattaagcggccgcctcgaggacgtcaggtaggccttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactggtcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgggaggcctgagtacttaatacgactcactatagg